(19) United States
(12) Patent Application Publication
(10) Pub. No.: US 2011/0250199 A1
(43) Pub. Date: Oct. 13, 2011

(54) IMMUNOTOXINS AND USES THEREOF

(75) Inventors: David J. Fitzgerald, Rockville, MD (US); Robert Sarnovsky, Phoenix, MD (US)

(73) Assignee: The Government of the United States of America as represented by the Secretary of the Department...., Rockville,, MD (US)

(21) Appl. No.: 12/996,138

(22) PCT Filed: Jun. 4, 2009
(86) PCT No.: PCT/US2009/046292

§ 371 Date: Jun. 30, 2011

Related U.S. Application Data
(60) Provisional application No. 61/058,872, filed on Jun. 4, 2008.

(51) Int. Cl. A61K 39/395; C12N 5/071; A61K 38/16; A61P 35/00; C07K 14/28; C07K 16/28

(52) U.S. Cl. 424/134.1; 530/350; 530/387.3; 514/1.1; 435/375; 435/366

(57) ABSTRACT

The invention provides novel recombinant immunotoxins comprising domain III of cholix toxin and exotoxin from Vibrio cholerae. The present invention further provides methods for using the compositions of the present invention to (i) induce apoptosis in a cell bearing one or more surface markers (ii) inhibit unwanted growth, hyperproliferation or survival of a cell bearing one or more cell surface markers, (iii) treat a condition, such as a cancer, (iv) provide therapy for a mammal having developed antibodies to Pseudomonas exotoxin A, and (v) provide therapy for a mammal having developed a disease caused by the presence of cells bearing one or more cell surface marker.

### CROSS-REFERENCES TO RELATED APPLICATIONS

This application claims benefit of U.S. provisional application Ser. No. 61/058,872, filed Jun. 4, 2008, the disclosure of which is incorporated herein in its entirety.

### FIELD OF THE INVENTION

The present invention relates generally to toxins, and targeted toxins, more specifically to antibody-toxin fusion proteins, referred to as immunotoxins. Toxins and targeted toxins comprise cholix toxin (CT), cholera exotoxin (CET) and Pseudomonas exotoxin (PE). Immunotoxins of the present invention can be used to treat cancer and other malignancies.

### BACKGROUND OF THE INVENTION

Antibody-based therapies of human cancer have become first line treatments in certain settings. By way of example, Her2-positive breast cancer patients are treated with Herceptin (Hudis, 2007, N Engl J Med 357:39-51) while individuals with certain B-cell malignancies receive Rituxan (Cheson and Leonard, 2008, N Engl J Med 359:613-26). These antibodies are given either alone or in combination with chemotherapy. The potential benefit of using antibody-based therapy is an effective treatment with low side effects. However, when the administration of an unmodified antibody is not effective, several options are available to make the antibody a ‘cytotoxic’ agent (Heimann and Weiner, 2007, Surg Oncol Clin N Am 16:775-92, viii). Radionuclides, small molecular weight drugs (including prodrugs), enzymes, homing partners (such as bispecific antibodies) and protein toxins have each been “attached” to tumor-binding antibodies as adjuncts to increase their effectiveness (Green et al., 2007, Clin Cancer Res 13:5598s-603s; Rybak, 2008, Curr Pharm Biotechnol 9:226-30; Liu et al., 2008, Immunological Reviews 222:9-27; Singh et al., 2008, Curr Med Chem 15:1802-26; Brumlik et al., 2008, Expert Opin Drug Deliv 5:87-103; Carter and Senter, 2008, Cancer J 14:154-69; Goldenberg and Sharkey, 2007, Oncogene 26:3734-44; Pastan et al., 2007, Annu Rev Med 58:221-37; Kreitman and Pastan, 2006, Hematol Oncol Clin North Am 20:1137-51, viii). Each type of modified antibody has benefits and limitations (Heimann and Weiner, 2007, Surg Oncol Clin NAm 16:775-92, viii; Ricart and Tolcher, 2007, Nat Clin Oncol 4:245-55).

In the past several years immunoconjugates have been developed as an alternative therapeutic approach to treat malignancies. Immunoconjugates were originally composed of an antibody chemically conjugated to a plant or a bacterial protein toxin, a form that is known as an immunotoxin. The antibody binds to the antigen expressed on the target cell and the toxin is internalized, arresting protein synthesis and inducing cell death (Brinkmann, U., Mol. Med. Today, 2:439-446 (1996)). More recently, genes encoding the antibody and the toxin have been fused and the immunotoxin expressed as a fusion protein.

A number of plant and bacterial toxins have been studied for their suitability as the toxin component of immunotoxins. For example, the bacterial toxin known as Pseudomonas exotoxin A (“PE”) has been studied for two decades as a toxin for use in immunotoxins. Typically, PE has been truncated or mutated to reduce its non-specific toxicity while retaining its toxicity to cells to which it is targeted by the antibody portion of the immunotoxin. Over the years, numerous mutated and truncated forms of PE have been developed and clinical trials employing some of them are ongoing.

Bacterial protein toxins are well known in the art, and are discussed in such sources as Burns, D., et al., eds., BACTERIAL PROTEIN TOXINS, ASM Press, Herndon Va. (2003), Aktories, K. and Just, I., eds., BACTERIAL PROTEIN TOXINS (HANDBOOK OF EXPERIMENTAL PHARMACOLOGY), Springer-Verlag, Berlin, Germany (2000), and Alouf, J. and Popoff, M., eds., THE COMPREHENSIVE SOURCEBOOK OF BACTERIAL PROTEIN TOXINS, Academic Press, Inc., San Diego, Calif (3rd Ed., 2006).

A number of the bacterial protein toxins act as ADP-ribosyltransferases. In the case of Pseudomonas exotoxin A (PE) and diphtheria toxin (DT), the ADP-ribosylation is of elongation factor 2 in eukaryotic cells. Since EF-2 is essential for protein synthesis in eukaryotic cells, inactivation of the EF-2 in a eukaryotic cell causes death of the cell. The sequences and structure of PE and DT are well known in the art. Mutated fatins of DT suitable for use in immunotoxins are known in the art. See, e.g., U.S. Pat. Nos. 5,208,021 and 5,352,447. DT does not share significant sequence identity or structural similarity with PE. Since most persons in the developed world have been immunized against diphtheria, DT-based immunotoxins can generally only be used in compartments of the body, such as the brain, that cannot be accessed by antibodies.

The PE-based immunotoxins currently in clinical trials are highly immunogenic. This has proven to be less of a problem in the treatment of hematological malignancies, in which the ability of the immune system to mount a response is often compromised. Immunotoxins can typically be administered multiple times to patients with hematological malignancies. Even so, neutralizing antibodies are made in approximately 25% of these patient. Patients with solid tumors, however, usually (>90%) develop neutralizing antibodies to PE-based immunotoxins within weeks after the first administration. Since many protocols call for a three week period between administration of immunotoxins, the development of the antibodies during this period effectively means that, for solid tumors, usually only one administration can be made of a PE-based immunotoxin before the patient develops antibodies which render it ineffective.

A number of bacterial toxins are ADP-ribosyltransferases. Two, Pseudomonas exotoxin A (“PE”) and diphtheria toxin (“DT”), irreversibly ribosylate elongation factor 2 (“EF-2”) in eukaryotic cells, causing the death of affected cells by inhibiting their ability to synthesize proteins. The PE-based targeted toxins currently in clinical trials are immunogenic and in many protocols can only be given once before the patient develops neutralizing antibodies, rendering further administrations of little use.

Jorgensen, R. et al., J Biol Chem 283(16):10671-10678 (2008) (hereafter, “Jorgensen”) recently reported that some strains of Vibrio cholerae, the causative agent of cholera, contain a ADP-ribosyltransferase, which they termed cholix toxin (also referred to herein as “CT”). Like PE, CT ribosylates EF-2. Jorgensen stated that CT's primary structure shows a 32% sequence identity with PE, and has a potential furin protease cleavage site for cellular activation, like that of PE, and contains a C-terminal KDEL sequence (SEQ ID NO:4), similar to the C-terminal sequence of PE, that likely targets the toxin to the endoplasmic reticulum of the host cell (Jorgensen, at page 10673). Jorgensen further reports that CT, like PE, is organized in three structural domains: domain Ia (residues 1-264), a receptor binding domain, a short domain Ib (residues 387-423), of unknown function, which with domain Ia comprise “a 13-stranded antiparallel β-jellyroll”, domain II (residues 265-386), a translocation domain consisting of six α-helices, and domain III, a catalytic domain with an α/β topology (Jorgensen, at page 10675). In fact, FIG. 3b of Jorgensen superpositions the structures of CT and PE, showing that the two structures are almost indistinguishable from one another.

Recombinant immunotoxins are antibody-based therapeutics typically composed of Fv fragments fused with protein toxins (Pastan et al., 2007, Annu Rev Med 58:221-37; Frankel et al., 2000, Clin Cancer Res 6:326-34; Frankel et al., 2003, Semin Oncol 30:545-57; Pastan et al., 2006, Nat Rev Cancer 6:559-65). As described above, the protein toxins are usually derived from bacterial or plant cytotoxic proteins and act enzymatically within the cytosol of mammalian cells. Advantages of toxin-based agents relate to their potency, lack of mutagenic activity and the fact that cancer cells rarely exhibit toxin resistance. The main disadvantage is their immunogenicity (Schnell et al., 2003, Ann Oncol 14:729-36; Schnell et al., 2002, Clin Cancer Res 8:1779-86; Frankel, 2004, Clin Cancer Res 10:13-5; Messmer and Kipps, 2005, Breast Cancer Res 7:184-6; Onda et al., 2008, Proc Natl Acad Sci USA 105:11311-6; Onda et al., 2006, J Immunol 177:8822-34; Posey et al., 2002, Clin Cancer Res 8:3092-9; Weldon et al., 2009, Blood 113(16):3792-800). Pseudomonas exotoxin (PE) has been investigated for a number of years as the cytotoxic partner to antibody fragments in the development of anticancer immunotoxins (Kreitman and Pastan 2006, Hematol Oncol Clin North Am 20:1137-51, viii; Pastan et al., 2006, Nat Rev Cancer 6:559-65). Typically, a 38 kDa fragment of PE, encompassing domains II and III of the parental toxin, is fused genetically to either a single chain Fv (scFv) or disulfide stabilized Fv.

With the exception of treating individuals with B-cell malignancies (Kreitman et al., 2001, N Engl J Med 345:241-7), most immunotoxin trials have been limited to one or sometimes two cycles of therapy because patients develop neutralizing antibodies, usually within three weeks of initiating treatment (Posey et al., 2002, Clin Cancer Res 8:3092-9; Hassan et al., 2007, Clin Cancer Res 13:5144-9). Potential strategies to make such toxin-based agents less immunogenic include the co-administration of immunosuppressive agents (Hassan et al., 2004, Clin Cancer Res 10:16-8; Knechtle, 2001, Philos Trans R Soc Lond B Biol Sci 356:681-9; Pai et al., 1990, Cancer Res 50:7750-3) and the re-engineering of the parent molecule to remove major epitopes (Onda et al., 2008, Proc Natl Acad Sci USA 105:11311-6; Weldon et al., 2009, Blood 113(16):3792-800). While the co-administration of immunosuppressive agents is simple in concept, it is difficult to accomplish in Phase I and II trials due to the confounding problem of mixing two agents where the properties of one, in this case the immunotoxin, are not well understood. The prospect of engineering a bacterial toxin to render it non-immunogenic is also challenging. However, by removing the most potent antigenic epitopes, it may be possible to administer several cycles of therapy before a neutralizing response develops (Onda et al., 2008, Proc Natl Acad Sci USA 105:11311-6).

Applicants reasoned that the replacement of the toxin portion of an immunotoxin with a closely related but immunologically distinct ‘molecular cousin’ may allow for a third approach. This strategy should work best in situations where structural similarities are close enough to allow for domain swapping and the use of a ‘modular replacement strategy’. In an attempt to obtain a more active toxin which may also be less immunogenic than CT, applicants have focused on a cholera exotoxin which is conserved among patient samples and is similar to, but different from the CT isolated by Jorgensen which was isolated from an environmental strain. Applicants herein refer to this cholera exotoxin as “CET.” Given the close structural similarity of PE, CT and CET and the high degree of sequence homology, it appeared likely that antibodies to conformational epitopes of PE would cross-react with CT and CET, while antibodies to non-conformational epitopes would cross-react due to the significant sequence identity of the two toxins, rendering CT- and CET-based targeted toxins unusable as a follow-on therapy for patients who have developed anti-PE antibodies following administration of PE-based chimeric proteins. Surprisingly, the studies underlying the invention show that the CET-based toxins are functionally similar to PE toxins, but immunologically distinct.

### BRIEF SUMMARY OF THE INVENTION

In light of the results described herein, CT- and CET-based targeted toxins, such as immunotoxins, can be used to provide one or more rounds of therapy to a mammal which has already developed neutralizing antibodies to PE. Alternatively, in light of the results described herein, CT- and CET-based targeted toxin, such as immunotoxins, can be used to provide one or more rounds of therapy in a mammal prior to administration of one or more PE-based targeted toxins. Thus, CT- and CET-based targeted toxins, such as immunotoxins, can be used either as a second line therapy in patients previously treated with a PE-based targeted toxin, such as an immunotoxin, or as a first-line therapy to be followed by therapy with a PE-based targeted toxin.

It is further expected that the ability to provide one or more rounds of targeted toxin therapy afforded by the availability of CT- and CET-based targeted toxins alone or in combination with PE-based targeted toxins will enhance the ability of practitioners to slow or stop the progression of disorders caused by the presence of cells which are the targets of the targeted toxins.

Thus, the present invention provides compositions comprising isolated toxins, in particular targeted toxins, such as immunotoxins compromising PE, CT, or CET, methods of making them and methods for their use.

In one aspect of the present invention, isolated toxins are provided. A preferred isolated toxin comprises a domain III of cholera exotoxin (CET) having an amino-terminal sequence and a carboxy-terminal sequence, and at least 65% sequence identity to an amino acid sequence of SEQ ID NO:36. Other preferred isolated toxins are isolated toxins wherein domain III is selected from a group consisting of a CET domain III having greater than about 85% sequence identity to an amino acid sequence of SEQ ID NO:36, a CET domain III having greater than about 90% sequence identity to an amino acid sequence of SEQ ID NO:36, a CET domain III having greater than about 91% sequence identity to an amino acid sequence of SEQ ID NO:36, a CET domain III having greater than about 92% sequence identity to an amino acid sequence of SEQ ID NO:36, a CET domain III having greater than about 93% sequence identity to an amino acid sequence of SEQ ID NO:36, a CET domain III having greater than about 94% sequence identity to an amino acid sequence of SEQ ID NO:36, a CET domain III having greater than about 95% sequence identity to an amino acid sequence of SEQ ID NO:36, a CET domain III having greater than about 96% sequence identity to an amino acid sequence of SEQ ID NO:36, a CET domain III having greater than about 97% sequence e identity to an amino acid sequence of SEQ ID NO:36, a CET domain III having greater than about 98% sequence identity to an amino acid sequence of SEQ ID NO:36, and a CET domain III having greater than about 99% sequence identity to an amino acid sequence of SEQ ID NO:36.

Preferably, the isolated toxin further comprises at least one of amino acid residues 253E, 283R, 352A, or 359Q of SEQ ID NO:2.

In a preferred embodiment, an isolated toxin further comprises a furin cleavage sequence having an amino-terminal sequence and a carboxy-terminal sequence. In some embodiments, the carboxy-terminal sequence of the furin cleavage sequence is fused to the amino-terminal sequence of the CET domain III.

In some embodiments, the furin cleavage sequence of the toxin is a CET furin cleavage sequence. In some embodiments, the furin cleavage sequence is a Pseudomonas exotoxin A furin cleavage sequence.

Some preferred isolated toxins comprise a NAD binding site. The NAD binding site can be from CET or from PE.

Also preferred is an isolated toxin wherein domain III of CET comprises an amino acid sequence of SEQ ID NO:36 or a conservatively modified fragment thereof and wherein the toxin has cytotoxic activity

An isolated toxin can be a CET40 having an amino acid sequence of at least 85% identity to SEQ ID NO:24. Preferably, this toxin further comprises at least one of amino acid residues selected from the group consisting of 26P, 73A, 76Q, 107I, 131P, 254E, 284R, 353A, and 360Q of SEQ ID NO:24.

A preferred isolated toxin is a CET40 having an amino acid sequence of SEQ ID NO:24.

In some embodiments, the carboxy-terminal sequence of the CET domain III of the toxin is REDLK (SEQ ID NO:5).

In some embodiments, the CET domain III comprises amino acid residues corresponding to amino acid residues 293 and 294 of SEQ ID NO:1 which are selected from the group consisting of: D293G-L294W, D293D-L294W, and D293G-L294L.

In some embodiments, a toxin is a targeted toxin. A targeted toxin further comprises a targeting moiety which specifically binds to one or more cell surface markers. The targeting moiety is fused in frame to the toxin. Preferably, the cell surface marker is a cell surface receptor. Cell surface receptor that can be targeted using a toxin of the present invention include, but are not limited to, transferrin receptor, EGF receptor, CD19, CD22, CD25, CD21, CD79, mesothelin and cadherin.

The targeting moiety can be an antibody or an antibody fragment specifically binding to one or more cell surface markers. Antibody fragment may be selected from the group consisting of a Fab, a Fab′, a F(ab′)2, a scFv, a Fv fragment, a helix-stabilized antibody, a diabody, a disulfide stabilized antibody, and a domain antibody. A preferred antibody fragment is a scFv.

A preferred targeted toxin specifically binds to a transferrin receptor. A preferred toxin binding to a transferrin receptor comprises an amino acid sequence of SEQ ID NO:19.

Other targeted toxins of the present invention comprise as a targeting moiety a ligand that specifically binds to one or more cell surface markers.

In another aspect of the present invention methods of inhibiting growth of a population of cells bearing one or more cell surface markers are provided. In a preferred embodiment, this method comprises the step of contacting a population of cells with an isolated toxin of the present invention. Thereby the growth of the population of cells is inhibited.

In some embodiments, the method inhibiting the growth of a population of cells further comprises the step of contacting the population of cells with a second isolated toxin comprising (i) a targeting moiety which specifically binds at least one of the surface markers and (ii) a Pseudomonas exotoxin A (PE) toxin. In some embodiments, the step of contacting the population of cells with the second isolated toxin is performed prior to contacting the population of cells with the first isolated toxin.

In some embodiments, the first isolated protein is administered to said population of cells about three weeks after administration of the second isolated protein to the population of cells. In some embodiments, the first isolated protein is administered to the population of cells within about one month of administration of the second isolated protein to the population of cells. In some embodiments, the first isolated protein is administered to the population of cells within about two months of administration of the second isolated protein to the population of cells.

In other embodiments, the method of inhibiting the growth of a population of cells comprises the step of (a) contacting the population of cells with a first chimeric toxin comprising (i) a targeting moiety which specifically binds at least one of the surface markers and (ii) a toxin selected from a PE, a CT and a CET and (b) contacting the population of cells with a second chimeric toxin comprising (i) a second targeting moiety which specifically binds at least one of the surface markers and (ii) a toxin selected from a PE, a CT and a CET, wherein the toxin of the second chimeric protein is not the same toxin comprising part of the first chimeric protein. Thereby the growth of the population of cells is inhibited. The first and second targeting moieties may bind to the same or different cell surface markers.

In some embodiments, the targeting moiety of the first and the second isolated or chimeric proteins specifically bind to the same cell surface marker. In some embodiments, the targeting moiety of the first and the second isolated or chimeric proteins is the same.

In some embodiments, the toxin of the first chimeric protein is PE and the toxin of the second chimeric protein is CET. In some embodiments, the toxin of the first chimeric protein is CET and the toxin of the second chimeric protein is PE.

A preferred PE is a PE40. A preferred PE40 comprises an amino acid sequence of SEQ ID NO:25 or a conservatively modified cytotoxic variant thereof.

A preferred CET is a CET40. A preferred CET40 comprises an amino acid sequence of SEQ ID NO:24 (FIG. 9B) or a conservatively modified cytotoxic variant thereof. Another preferred CET is a CET having an amino acid sequence of SEQ ID NO:2 or a conservatively modified cytotoxic variant thereof.

A preferred isolated toxin for use in the methods of the present invention may comprise the NAD binding site of PE.

A first chimeric protein may be selected from the group consisting of an immunotoxin comprising an amino acid sequence of SEQ ID NO:16 (FIG. 2B), an immunotoxin comprising an amino acid sequence of SEQ ID NO:35, an immunotoxin comprising an amino acid sequence of SEQ ID NO:22 (FIG. 9A) or an immunotoxin comprising an amino acid sequence of SEQ ID NO:19 (FIG. 3B).

In some embodiments, a CET comprises a furin cleavage sequence having an amino-terminal sequence and a carboxy-terminal sequence and a CET domain III having an amino-terminal sequence and a carboxy-terminal sequence, in which the carboxy-terminal sequence of the furin cleavage sequence is fused on the amino-terminal sequence of the CET domain III.

Other preferred CETs are CETs comprising the NAD binding site of PE, i.e wherein the CET NAD binding site is replaced by the NAD binding site of PE.

Also preferred CETs are CETs wherein the C-terminal amino acid sequence KDELK (SEQ ID NO:8) of the CET domain III is replaced by the amino acid sequence REDLK (SEQ ID NO:5).

The population of cells preferably are mammalian cells, more preferably, human cells. Even more preferred are human disease cells or human malignant cells. A preferred malignant cell is selected from the group consisting of neuroblastoma, intestine carcinoma, rectum carcinoma, colon carcinoma, familiary adenomatous polyposis carcinoma, hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors, glioblastoma, astrocytoma, meningioma, medulloblastoma, peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroids melanoma, seminoma, rhabdomyosarcoma, craniopharyngeoma, osteosarcoma, chondrosarcoma, myosarcome, liposarcoma, fibrosarcoma, Ewing sarcoma, and plasmocytoma.

The cell surface marker can be a cell surface receptor. Cell surface receptor that can be targeted using a toxin of the present invention include, but are not limited to, transferrin receptor, EGF receptor, CD19, CD22, CD25, CD21, CD79, mesothelin and cadherin. In some embodiments, the cell surface marker is mesothelin. In some embodiments, the cell surface marker is CD22.

In another aspect of the present invention, a method of providing therapy for a mammal having developed neutralizing antibodies to Pseudomonas exotoxin A is provided. In a preferred embodiment, this method comprises the steps of (a) selecting a mammal having developed neutralizing antibodies to Pseudomonas exotoxin A and (b) administering to said mammal an isolated toxin or targeted toxin of the present invention.

Also provided is a method a method of providing therapy for a mammal having developed a disease caused by the presence of cells bearing one or more cell surface markers. In a preferred embodiment, this method comprises the steps of (a) administering to said mammal a first isolated toxin or first targeted toxin of the present invention. and (b) administering to said mammal a second isolated toxin or second targeted toxin comprising (i) a targeting moiety which specifically binds to at least one surface marker on said cells and (ii) Pseudomonas exotoxin A toxin. Step (a) of this method can be performed before or after step (b).

### BRIEF DESCRIPTION OF THE DRAWINGS

FIG. 1 depicts an alignment of the sequences of (a) a 40 kD Pseudomonas exotoxin A (PE) fragment (amino acid residues 252-613) known as “PE40” (SEQ ID NO:13), which consists of domains II, Ib, and III of PE, and (b) a 40 kD fragment of Cholera exotoxin (amino acid residues 264-634) referred to in the Figure as “CholExo” (SEQ ID NO:14), which consists of domains II, Ib, and III of that toxin. Areas of the greatest similarity are boxed. Asterisks denote identical residues. Dots denote conservative substitutions.

FIG. 2A depicts a schematic diagram of the plasmid pHB21+PE38, encoding a chimeric protein comprising an anti-transferrin receptor single chain Fv antibody fragment known as HB21, fused to PE38. “CAT” is an antibiotic resistance gene used to facilitate identifying selecting cells successfully transfected with the plasmid. FIG. 2B depicts the sequence of the plasmid (SEQ ID NO:15). Restriction sites and translation of the encoded sequences (SEQ ID NOS:16 and 17) are shown. The amino acid sequence of HB21scFv-PE38 is shown in SEQ ID NO:16.

FIG. 3A depicts a schematic diagram of the plasmid pHB21+CET40 encoding a chimeric protein comprising an anti-transferrin receptor antibody known as HB21 fused to a 40 kD truncated form of Cholera exotoxin (amino acid residues 270-634). (In applicants' laboratory, the plasmid pHB21+CET40, previously was referred to as “phB21+VcPE40” having the identical sequence as pHB21+CET40). “CAT” is an antibiotic resistance gene used to facilitate identifying cells successfully transfected with the plasmid. FIG. 3B depicts the sequence of the plasmid (SEQ ID NO:18). Restriction sites and translation of the encoded sequences (SEQ ID NOS:19 and 17) are shown. The amino acid sequence of HB21scFv-CET40 is shown in SEQ ID NO:19.

FIG. 4 depicts neutralization of PE40 immunotoxin with rabbit anti-PE and M40-1 antibodies. A graph showing the effect of contacting cells expressing transferrin receptor as a cell surface marker with an immunotoxin comprising an anti-transferrin receptor antibody, HB21, fused to a 40 kD form of Pseudomonas exotoxin A referred to as “PE40”, in the presence or absence of polyclonal or monoclonal anti-PE antibodies is shown. Immunotoxin and antibody were pre-mixed for 30 minutes at room temp and the mixture added to Ddl-1 human colon cancer cells. Cells were incubated for 48 hrs and then assessed for viability using a WST-1 cell proliferation assay. Y axis: shows optical absorbance at 450 nm, with higher values on the axis showing higher levels of cell growth and proliferation. X axis: states the amounts of immunotoxin or anti-PE antibody, or both, present with respect to the experiment whose results are depicted in the bar above the statement. HB21-PE40=PE40-based anti-transferrin receptor immunotoxin HB21scFv-PE40. Rabbit anti-PE=polyclonal rabbit antibodies raised against native PE. M40-1=a monoclonal anti-PE antibody. Cycloheximide=protein synthesis inhibitor used as a positive control. Details are described in Example 5.

FIG. 5 depicts the activity of HB21-CET40 treated with polyclonal (rabbit) and monoclonal (M40-1) anti-PE antibodies. A graph showing the effect of contacting cells expressing transferrin receptor as a cell surface marker with an immunotoxin comprising an anti-transferrin receptor antibody, HB21, fused to a 40 kD truncated folin of Cholera exotoxin (“CET”), in the presence or absence of polyclonal or monoclonal anti-PE antibodies is shown. Testing was as described for FIG. 4. Y axis: shows optical absorbance at 450 nm, with higher values on the axis showing higher levels of cell growth and proliferation. X axis: states the amounts of immunotoxin or anti-PE antibody, or both, present with respect to the experiment whose results are depicted in the bar above the statement. HB21-CET40=CET40-based anti-transferrin receptor immunotoxin HB21scFv-CET40. Rabbit anti-PE=polyclonal rabbit antibodies raised against native, folinaldehyde-treated PE. M40-1=a monoclonal anti-PE antibody. Cycloheximide=protein synthesis inhibitor used as a positive control. Details are described in Example 6.

FIG. 6 depicts neutralization of PE40 immunotoxin with a commercially available antibody. A graph showing the effect of contacting cells expressing transferrin receptor as a cell surface marker with an immunotoxin comprising an anti-transferrin receptor antibody, HB21, fused to the 40 kD cytotoxin known as PE40, in the presence or absence of an anti-PE polyclonal antibody commercially available from Sigma. Testing was as described for FIG. 4. Y axis: shows absorbance at 450 nm, with higher values on the axis showing higher levels of cell growth and proliferation. X axis: states the amounts of immunotoxin or anti-PE antibody, or both, present with respect to the experiment whose results are depicted in the bar above the statement. HB21-PE40=PE-40-based anti-transferrin receptor immunotoxin HB21scFv-PE40. Sigma anti-PE=Sera containing rabbit anti-PE polyclonal antibodies raised against PE, purchased from Sigma. Normal rabbit sera=sera from non-immunized animals, as negative control. Sigma sera=Sera containing rabbit anti-PE polyclonal antibodies raised against PE, purchased from Sigma. Cycloheximide=protein synthesis inhibitor used as a positive control. Details are described in Example 7.

FIG. 7 depicts neutralization of CET40 immunotoxin with a commercially available antibody. A graph showing the effect of contacting cells expressing transferrin receptor as a cell surface marker with an immunotoxin comprising an anti-transferrin receptor antibody, HB21, fused to a 40 kD truncated form of Cholera exotoxin (“CET40”), in the presence or absence of a anti-PE polyclonal antibody commercially available from Sigma. Testing was as described for FIG. 4. Y axis: shows absorbance at 450 nm, with higher values on the axis showing higher levels of cell growth and proliferation. X axis: states the amounts of immunotoxin or anti-PE antibody, or both, present with respect to the experiment whose results are depicted in the bar above the statement. HB21-CET40=CET40-based anti-transferrin receptor immunotoxin HB21scFv-CET40. Sigma anti-PE=Sera containing rabbit anti-PE polyclonal antibodies raised against PE, purchased from Sigma. Normal rabbit sera=sera from non-immunized animals, as negative control. Sigma sera=Sera containing rabbit anti-PE polyclonal antibodies raised against PE, purchased from Sigma. Cycloheximide=protein synthesis inhibitor used as a positive control. Details are described in Example 8.

FIG. 8 depicts a photograph of Western blots conducted with approximately 25 ng of purified immunotoxins. HB21-CET40: an anti-transferrin receptor antibody, HB21, fused to a 40 kD truncated form of Cholera exotoxin (“CET”), HB21scFv-CET40. HB21-PE40: same antibody, fused to 40 kD truncated form of Pseudomonas exotoxin A, HB21scFv-PE40. M40-1=a monoclonal anti-PE antibody. Sigma anti-PE: Sera containing rabbit anti-PE polyclonal antibodies raised against PE, purchased from Sigma. Rabbit anti-PE=polyclonal rabbit antibodies raised against native, formaldehyde-treated PE. Details are described in Example 9.

FIG. 9A depicts the nucleotide sequence (SEQ ID NO:21) and deduced amino acid sequence (SEQ ID NO:22) of the immunotoxin HB21scFv-CET40 (comprising putative domains II and III of Cholera exotoxin, CET40). Shown is the DNA and protein sequence of the immunotoxin HB21scFv-CET40 (HB21_CET40GENE). The initiating methionine is followed by the variable portion of the heavy chain, a glycine-serine linker [GGGGSGGGGSGGGGS (SEQ ID NO:26), the variable portion of the light chain, a short connector sequence (including the HindIII site and encoding ASGGP (SEQ ID NO:27) and amino acid residues 270-634 of cholera exotoxin.

FIG. 9B depicts a sequence alignment via ‘ClustalX’ (2.09) analysis. Shown in descending order are amino acids 270-634 of cholix toxin (cholix_II_III; SEQ ID NO:23) (Jorgensen et al., 2008, J Biol Chem 283:10671-8), amino acids 270-634 of CET (CET_II_III; SEQ ID NO:24) (corresponding to domains II, Ib, and III from the exotoxin gene isolated from a patient infected with Vibrio cholerae strain 1587; and finally domains II and III of exotoxin A PE40 (PE_II_III; SEQ ID NO:25). Key common features include the location of a furin cleavage sequence, showing amino acid residues 18 to 30 corresponding to P6, P5, P4, P3, P2, P1, P′1, P′2, P′3, P′4, P′S, P′6, P′7 (see text), an NAD binding site comprising an glutamic acid (“E”) and a KDEL (SEQ ID NO:4)-like motif at the C-terminus. For alignment: “*”=fully conserved; “:”=conserved within a ‘strong’ group; and “.”=conserved within a ‘weak’ group. The terms “strong” group and “weak” group are as defined by the ClustalX (2.09) software.

FIG. 9C depicts a sequence alignment of amino acids 1-634 of cholix toxin (cholix; SEQ ID NO:31) (Jorgensen et al., 2008, J Biol Chem 283:10671-8) and amino acids 1-634 of CET (CET; SEQ ID NO:1). The amino acid sequence of CET differs from that of cholix toxin in the following 14 amino acid positions: 90, (CT=H; CET=N), 213 (CT=M; CET=I), 245 (CT=V; CET=A), 266 (CT=G; CET=K), 270 (CT=S; CET=E), 295 (CT=T; CET=P), 342 (CT=D, CET=A), 345 (CT=R, CET=Q), 376 (CT=T, CET=I), 400 (CT=S; CET=P), 523, (CT=D; CET=E), 553 (CT=E; CET=R), 622 (CT=T; CET=A), and 629 (CT=R; CET=Q). Putative domains Ia, II, Ib, and III are indicated by boxes.

FIG. 10 depicts fractions of HB21scFv-CET40 eluted from TSK G3000 column. Fractions 19-30 are shown after electrophoresis through a 4-20% Tris-glycine precast gel under reducing and non-reducing conditions. Fractions 28 and 29, marked with an asterisk, were used for experiments described herein. Details are described in Examples 2 and 4.

FIG. 11 depicts cytotoxicity assay data of HB21scFv-CET40 (“HB21-CET40”) compared with HB21scFv-PE40 (“HB21-PE40”) in various cell lines. Immunotoxin concentrations from 0.1-100 ng/ml were added to each of four cell lines for 48 hr: A, A549 cells (lung); B, KB 3-1 cells (epidermoid); C, Raji cells (B-cell); and D, HUT102 cells (T-cell). Cells were used as representative cell lines of various common cancers. Cell viability was determined using the WST-1 reagent. Error bars represent one standard deviation (SD) of 5 replicate wells per data point. Details are described in Example 10.

FIG. 12 depicts cytotoxicity assay data of HB21scFv-CET40 (“HB21-CET40”) compared with HB21scFv-PE40 (“HB21-PE40”) in various cells. DLD-1 cells (colon; A) and 293TT cells (kidney; B) cells were assayed for susceptibility to immunotoxin killing. Cell viability was determined using the WST-1 reagent. Error bars represent one standard deviation (SD) of 5 replicate wells per data point. Details are described in Example 10.

FIG. 13 depicts immunotoxin specificity. A. Excess HB21 antibody competes for killing activity on DLD1 cells. Cells were pretreated or not with the HB21 antibody (10 μg/ml) for 1 hr at 37° C. and then incubated with HB21scFv-CET40 at 10 and 1 ng/ml. B. Immunotoxin activity on mouse cell line L929. HB21scFv-CET40 or HB21scFv-PE40 was added to L929 cells at concentrations from 0.1 to 100 ng/ml. Cell viability was assessed after 48 hr using the WST-1 reagent. Error bars represent one SD of 5 replicate wells per data point. Details are described in Example 10.

FIG. 14 depicts toxin reactivity via Western blot analysis. A. Western blot analysis of HB21scFv-PE40 (“HB21-PE40”) and HB21scFv-CET40 (“HB21-CET40”). Immunotoxins ˜30 ng per lane were separated on a reducing 8-16% Gel and transferred to a PVDF membrane. Immunotoxins and a lane with molecular weight (MW) markers were each probed with one of three anti-PE antibodies (from left to right: monoclonal antibody M40-1, rabbit anti-PE from Sigma-Aldrich and rabbit anti-PE raised at the National Cancer Institute (NCI)). B. Western blot analysis of CET and PE probed with anti-CET40 antibodies. CET or PE at 30 and 3 ng per lane were probed with a rabbit anti-HB21scFv-CET40 antibody preparation. Details are described in Example 11.

FIG. 15 depicts the neutralization activity of anti-PE antibody preparations. Rabbit anti-PE antibodies “Sigma” (A; Immunotoxin plus anti-PE serum (Sigma)) and Rabbit anti-PE antibodies “NCI” (B, Immunotoxin plus anti-PE serum (NCl)) were mixed with immunotoxins HB21scFv-PE40 (“HB21-PE40”) and (HB21scFv-CET40 (“HB21-CET40”) as indicated for 1 hr at room temp. At the end of the incubation 50 μl of the mixture was added to 50 μA of media over each well of DLD-1 cells. After a 48 hr, cell viability was assessed using the WST-1 reagent. Each bar represents a replicate of 5 with the error bar indicating one SD. Comparisons of immunotoxin activity without and with antibody incubations are indicated with lines. Details are described in Example 12.

FIG. 16 depicts the neutralizing activity of the monoclonal antibody M40-1 of the PE40-immunotoxin HB21sc-PE40 (A; “HB21-PE40”)) and the CET immunotoxin, HB21scFv-CET40 (B; “HB21-CET40”). Neutralizing activity was assessed via incubation with each immunotoxin as indicated followed by addition to DLD-1 cells. After a 48 hr incubation, cell viability was assessed using the WST-1 reagent. Each bar represents a replicate of 5 with the error bar indicating one SD. Comparisons of immunotoxin activity with and without M40-1 incubation are indicated with lines. Details are described in Example 12.

FIG. 17 depicts the neutralizing activity of pre and post-treatment sera from patients 1 and 2 treated with PE40-immunotoxin HB21scFv-PE40 (“HB21-PE40”) and CET immunotoxin HB21scFv-CET40 (“HB21-CET40”). A, PE40 Immunotoxin plus Patient Serum 1; B, CET40 Immunotoxin plus Patient Serum 1; C, PE40 Immunotoxin plus Patient Serum 2; D, CET40 Immunotoxin plus Patient Serum 2. Antisera (at 1:100) were mixed with either 5 or 1 ng/ml of immunotoxin as indicated for 1 hr at room temp. At the end of the incubation 50 μl of the mixture was added to 500 of media over each well in a 96-well format. After a 48 hr incubation, the viability of DLD-1 cells was assessed using a WST-1 reagent. Each bar represents a replicate of 5 with the error bar indicating one SD. Direct comparisons between the neutralization of HB21scFv-PE40 and HB21scFv-CET40 at 2.5 ng/ml are indicated with lines. Each experiment was conducted independently twice per patient sample. Details are described in Example 13.

FIG. 18 depicts the neutralizing activity of pre and post-treatment sera from patients 3 and 4 treated with PE40-immunotoxin HB21scFv-PE40 (“HB21-PE40”) and CET immunotoxin HB21scFv-CET40 (“HB21-CET40”). A, PE40 Immunotoxin plus Patient Serum 3; B, CET40 Immunotoxin plus Patient Serum 3; C, PE40 Immunotoxin plus Patient Serum 4; D, CET40 Immunotoxin plus Patient Serum 4. Antisera (at 1:100) were mixed with either 5 or 1 ng/ml of immunotoxin for 1 hr at room temp. At the end of the incubation 50 μl of the mixture was added to 50 μl of media over each well in a 96-well format. After a 48 hr incubation, the viability of DLD-1 cells was assessed using a WST-1 reagent. Each bar represents a replicate of 5 with the error bar indicating one SD. Direct comparisons between the neutralization of HB21scFv-PE40 and HB21scFv-CET40 at 2.5 ng/ml are indicated with lines. Each experiment was conducted independently twice per patient sample. Details are described in Example 13.

### DETAILED DESCRIPTION OF THE INVENTION

### I. Definitions

It must be noted that, as used in the specification and the appended claims, the singular forms “a,” “an,” and “the” include plural referents unless the context clearly dictates otherwise. Thus, for example, reference to “a compound” includes mixtures of compounds, reference to “a pharmaceutical carrier” includes mixtures of two or more such carriers, and the like.

As used throughout this application, the word “may” is used in a permissive sense (i.e., meaning having the potential to), rather than the mandatory sense (i.e., meaning must). Similarly, the words “include,” “including,” and “includes” mean including, but not limited to.

Recitation of ranges of values herein are merely intended to serve as a shorthand method of referring individually to each separate value falling within the range, unless otherwise indicated herein, each individual value is incorporated into the specification as if it were individually recited herein. Numeric ranges are inclusive of the numbers defining the range.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Units, prefixes, and symbols are denoted in their Systeme International de Unites (SI) accepted form. Unless otherwise indicated, nucleic acids are written left to right in 5′ to 3′ orientation; amino acid sequences are written left to right in amino to carboxy orientation.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., Rieger et al. (eds.), Springer Verlag (1991); and Hale & Marham, The Harper Collins Dictionary of Biology (1991). As used herein, the following tetins have the meanings ascribed to them unless specified otherwise.

For convenience of reference, as used herein, the term “antibody” includes whole (which may also be referred to as “intact”) antibodies, antibody fragments that retain antigen recognition and binding capability, whether produced by the modification of whole antibodies or synthesized de novo using recombinant DNA methodologies, monoclonal antibodies, polyclonal antibodies, and antibody mimics, unless otherwise required by context. The antibody may be an IgM, IgG (e.g. IgG₁, IgG₂, IgG₃ or IgG₄), IgD, IgA or IgE.

As used herein, the term “antibody fragment” refers to a molecule that comprises a portion of an intact antibody, generally the antigen binding or variable region of the intact antibody. Examples of antibody fragments include Fab, Fab′, F(ab′)₂, and Fv fragments; helix-stabilized antibodies (see, e.g., Arndt et al., J Mol Biol, 312:221-228 (2001)); diabodies (see below); single-chain antibody molecules (“scFvs,” see, e.g., U.S. Pat. No. 5,888,773); disulfide stabilized antibodies (“dsFvs”, see, e.g., U.S. Pat. Nos. 5,747,654 and 6,558,672), and domain antibodies (“dAbs,” see, e.g., Holt et al., Trends Biotech, 21(11):484-490 (2003), Ghahroudi et al., FEBS Lett., 414:521-526 (1997), Lauwereys et al., EMBO J, 17:3512-3520 (1998), Reiter et al., J Mol. Biol., 290:685-698 (1999), and Davies and Riechmann, Biotechnology, 13:475-479 (2001)).

As used herein, the teem “diabody” refers to a small antibody fragment with two antigen-binding sites, which fragments comprise a variable heavy domain (“V_{H}” or “VH”) connected to a variable light domain (“V_{L}” or “VL”) in the same polypeptide chain (V_{H}-V_{L}). By using a linker that is too short to allow pairing between the two domains on the same chain, the domains are forced to pair with the complementary domains of another chain and create two antigen-binding sites. Diabodies and their production are described more fully in, for example, EP 404,097; WO 93/11161; and Hollinger et al., Proc. Natl. Acad. Sci. USA, 90:6444-6448 (1993).

References to “V_{H}” or a “VH” refer to the variable region of an immunoglobulin heavy chain, including an Fv, scFv, dsFv or Fab. References to “V_{L}” or a “VL” refer to the variable region of an immunoglobulin light chain, including of an Fv, scFv, dsFv or Fab.

As used herein, the term “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

As used herein, the term “linker peptide” includes reference to a peptide within an antibody binding fragment (e.g., Fv fragment) which serves to indirectly bond the variable domain of the heavy chain to the variable domain of the light chain.

As used herein, the term “parental antibody” means any antibody of interest which is to be mutated or varied to obtain antibodies or fragments thereof which bind to the same epitope as the parental antibody, but with higher affinity.

As used herein, the term “hotspot” means a portion of a nucleotide sequence of a CDR or of a framework region of a variable domain which is a site of particularly high natural variation. Although CDRs are themselves considered to be regions of hypervariability, it has been learned that mutations are not evenly distributed throughout the CDRs. Particular sites, or hotspots, have been identified as these locations which undergo concentrated mutations. The hotspots are characterized by a number of structural features and sequences. These “hotspot motifs” can be used to identify hotspots. Two consensus sequences motifs which are especially well characterized are the tetranucleotide sequence RGYW and the serine sequence AGY, where R is A or G, Y is C or T, and W is A or T.

Typically, an immunoglobulin has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

As used herein, the term “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond.

As used herein, the term “disulfide stabilized Fv” or “dsFv” refers to the variable region of an immunoglobulin in which there is a disulfide bond between the light chain and the heavy chain. In the context of this invention, the cysteines which form the disulfide bond are within the framework regions of the antibody chains and serve to stabilize the conformation of the antibody. Typically, the antibody is engineered to introduce cysteines in the framework region at positions where the substitution will not interfere with antigen binding.

An antibody immunologically reactive with a particular antigen can be generated by recombinant methods such as selection of libraries of recombinant antibodies in phage or similar vectors, see, e.g., Huse et al., Science, 246:1275-1281 (1989); Ward, et al., Nature, 341:544-546 (1989); and Vaughan, et al., Nature Biotech., 14:309-314 (1996), or by immunizing an animal with the antigen or with DNA encoding the antigen.

As used herein, the terms “amino acid” or “amino acid residue” or “residue” include reference to an amino acid that is incorporated into a protein, polypeptide, or peptide (collectively “peptide”). The amino acid can be a naturally occurring amino acid and, unless otherwise limited, can encompass known analogs of natural amino acids that can function in a similar manner as naturally occurring amino acids.

As used herein, the Willis “attaching,” “conjugating,” “joining,” “bonding,” “fusing to,” “linking” or grammatical equivalents thereto refer to making two polypeptides into one contiguous polypeptide molecule. In the context of the present invention, the terms include reference to joining an antibody moiety to a PE of the invention. The linkage can be either by chemical or recombinant means. Chemical means refers to a reaction between the antibody moiety and the PE molecule such that there is a covalent bond formed between the two molecules to faun one molecule.

As used herein, the term “cell surface marker” refers to any antigen or receptor on the surface of a cell to which an antibody, an antibody fragment or ligand specifically binds.

As used herein, the ten “chimeric antibody” refers to an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

As used herein, the term “chimeric protein” refers to any single polypeptide unit that comprises two distinct polypeptide domains joined by a peptide bond, optionally by means of an amino acid linker, or a non-peptide bond, wherein the two domains are not naturally occurring within the same polypeptide unit. Typically, such chimeric proteins are made by expression of a cDNA construct but could be made by protein synthesis methods known in the art. A chimeric protein of the present invention contains, as a first polypeptide domain, an antibody or antibody fragment and, as a second polypeptide domain, a toxin. Such a chimeric protein can comprise a fragment or derivative of a naturally occurring antibody or a fragment or derivative of a naturally occurring toxin. A chimeric protein of the invention optionally contains a mimetic of the naturally occurring antibody or a mimetic of the naturally occurring toxin. In some embodiments, the distinct polypeptide domains can be in reverse orientation to those examples given herein, or in any order within the chimeric protein.

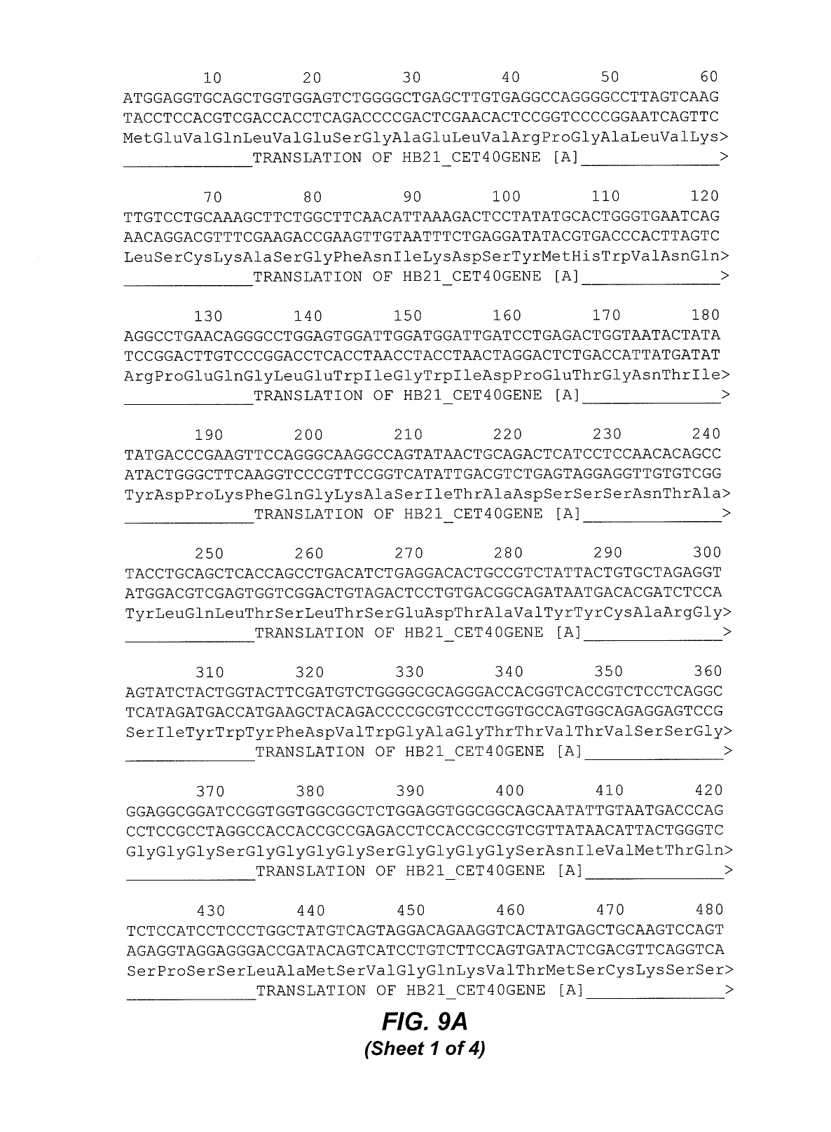

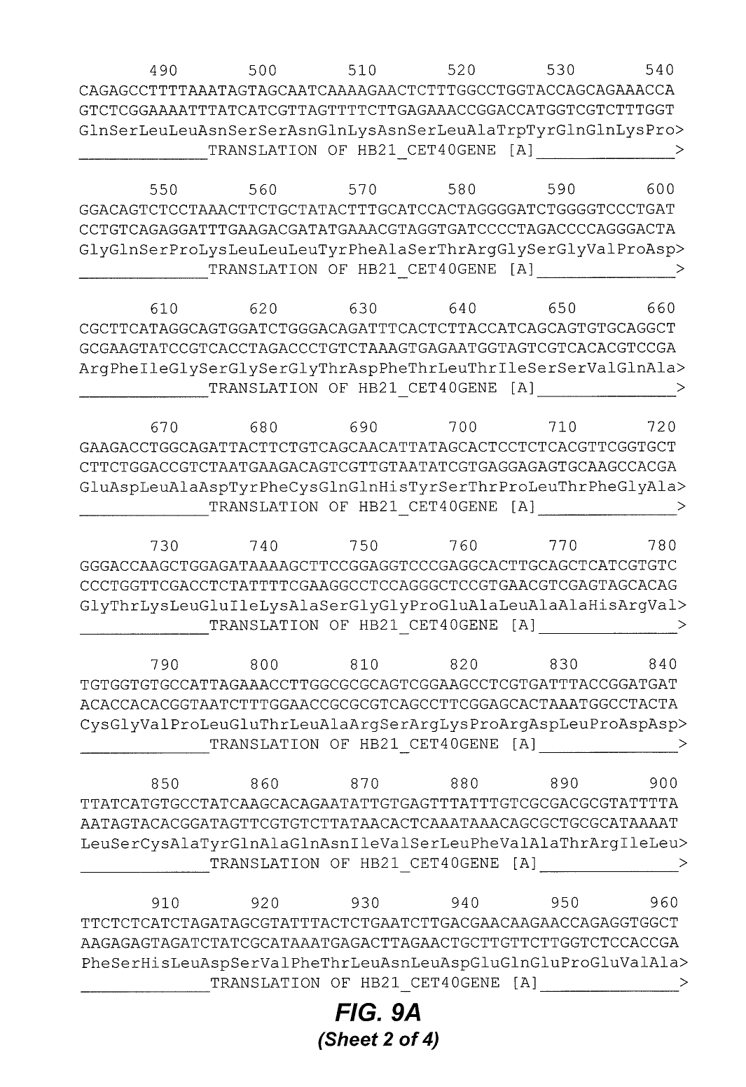

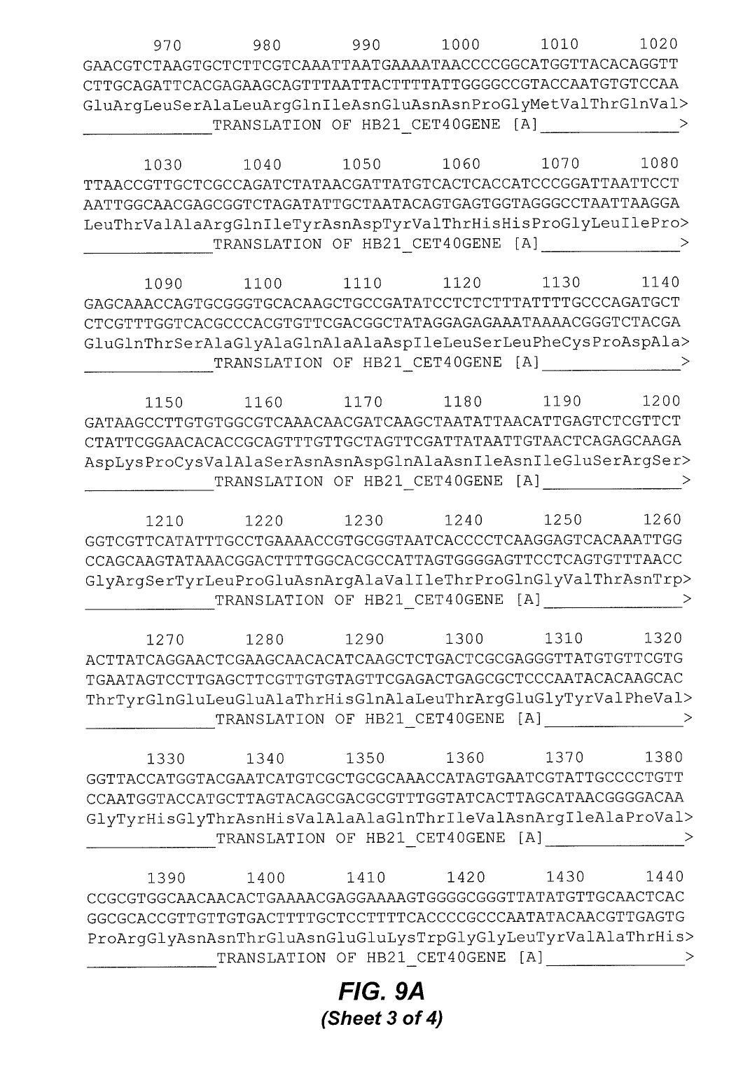

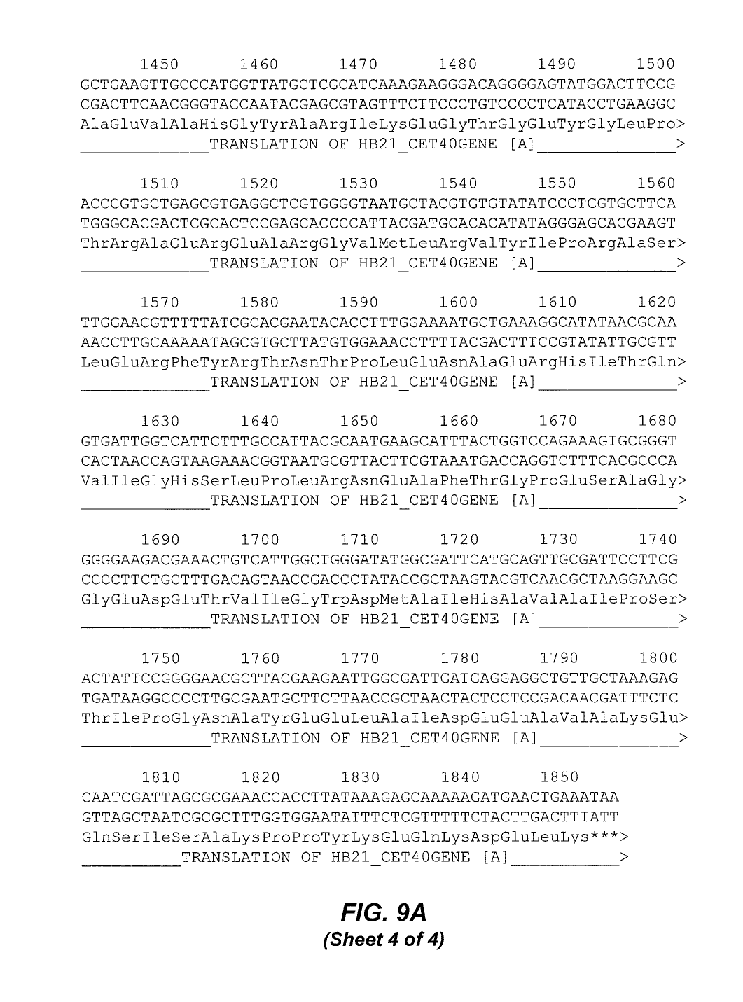

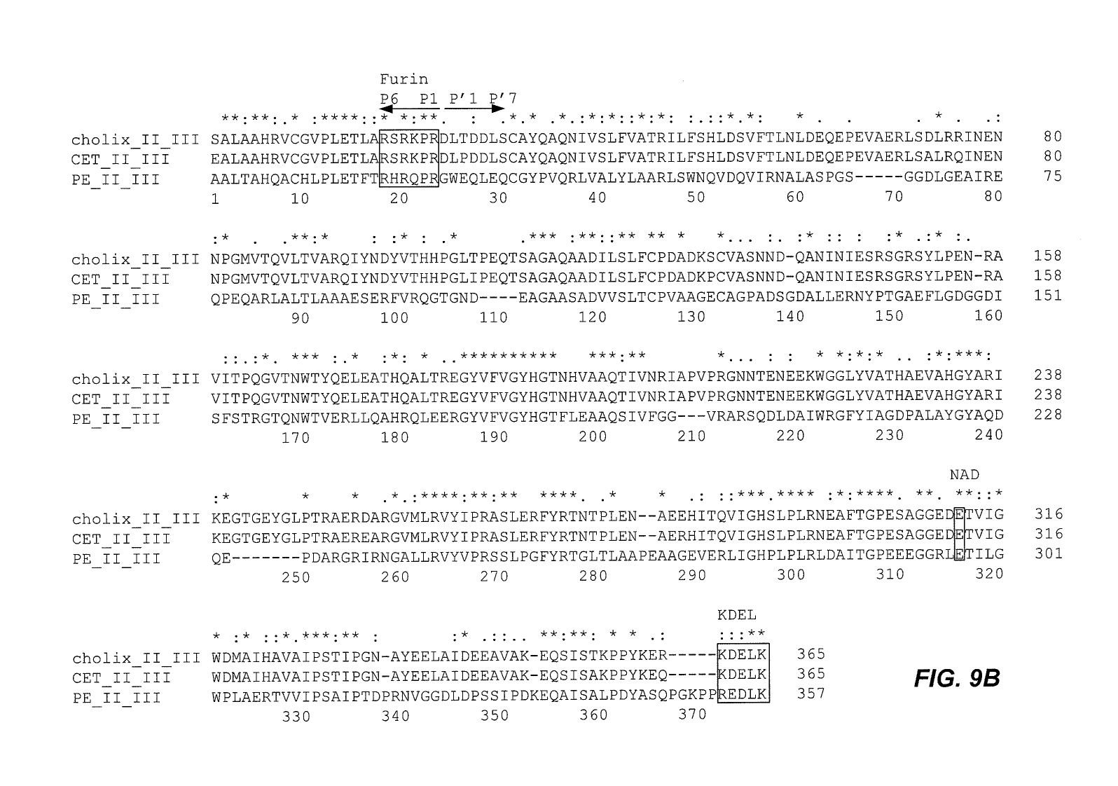

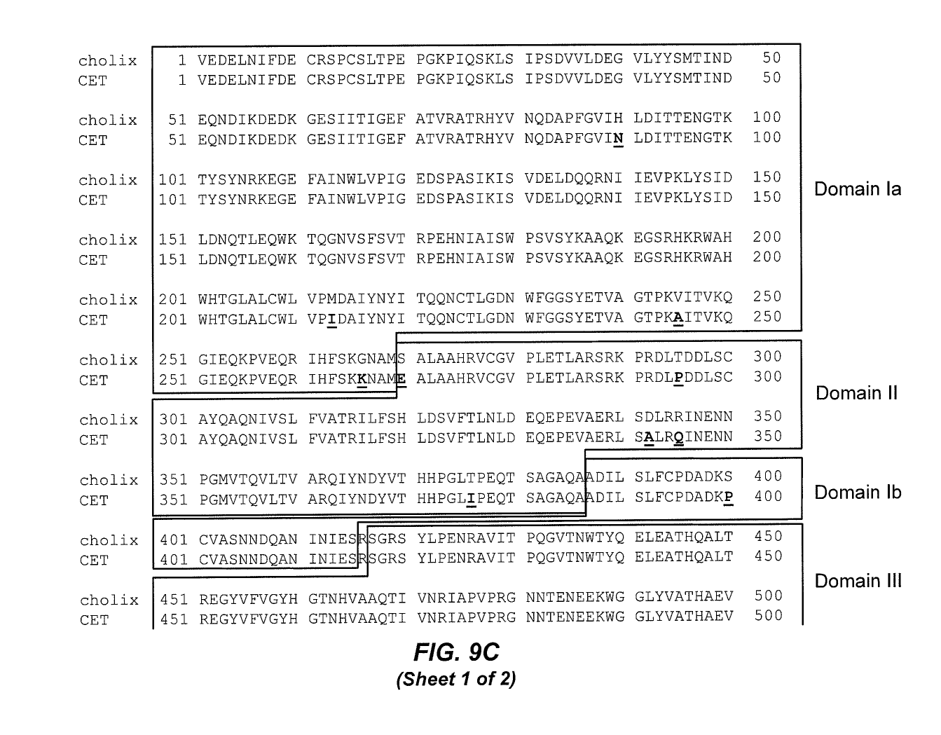

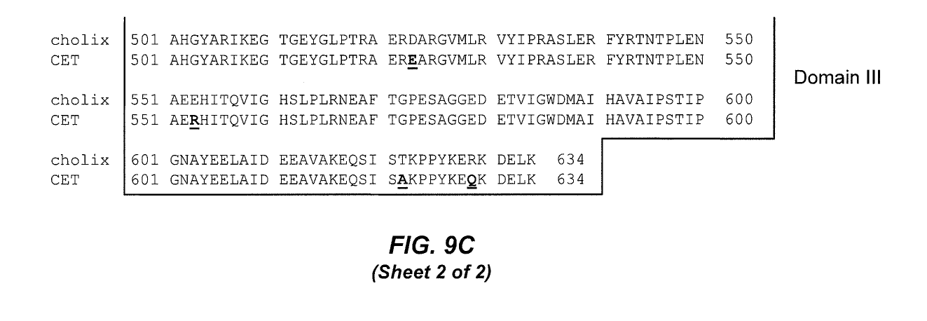

As used herein, the teams “Cholix toxin” or “CT” and “Cholera exotoxin” or “CET” refer to a toxin expressed by some strains of Vibrio cholerae that do not cause cholera disease. According to the article reporting the discovery of the Cholix toxin (Jorgensen, R. et al., J Biol. Chem. 283(16):10671-10678 (2008)), mature cholix toxin is a 70.7 kD, 634 residue protein, whose sequence is set forth as SEQ ID NO:31 and in FIG. 9C. The Jorgensen authors deposited in the NCBI Entrez Protein database a 642-residue sequence which consists of what they tellned the full length cholix toxin A chain plus, at the N-terminus an additional 8 residues, consisting of a 6 histidine tag flanked by methionine residues (SEQ ID NO:20), presumably introduced to facilitate expression and separation of the protein. The 642-residue sequence is available on-line in the Entrez Protein database under accession number 2Q5T_A and can be converted to the 634 amino acid sequence of SEQ ID NO:31 (FIG. 9C) by simply deleting the first 8 amino acids of the deposited sequence. Mature CT has four domains: Domain Ia (amino acid residues 1-269, as shown in FIG. 9C and in SEQ ID NO:31), Domain II (amino acid residues 270-386, as shown in FIG. 9C and in SEQ ID NO:31), Domain Ib (amino acid residues 387-415, as shown in FIG. 9C and in SEQ ID NO:31), and Domain III (amino acid residues 417-634, as shown in FIG. 9C and in SEQ ID NO:31). Mutations of CT will sometimes be described herein by reference to the amino acid residue present at a given position in the 634-amino acid sequence of native CT, even if the particular CT has been truncated to contain less than 634 residues. Thus, for example, the term “L294W” indicates that the “L” (leucine, in standard single letter code) residue at position 294 in native CT has been replaced by a “W” (tryptophan, in standard single letter code) in the mutated CT under discussion, even if the residue appears in a truncated CT. Similarly, reference to “L294” refers to a leucine residue at position 294 of the native CT sequence. For convenience of reference, the terms “cholix toxin” and CT″ as used herein may refer to the native or mature toxin, but more commonly refer to fomis in which the toxin has been modified to reduce non-specific binding, for example, by deletion of domain Ia, or otherwise improve its utility for use in immunotoxins. Which meaning is intended will be clear in context.

As used herein, the terms “Cholera exotoxin” or “CET” refer to a toxin expressed by some strains of Vibrio cholerae that do not cause cholera disease and include mature CET and cytotoxic fragments thereof. Mature cholera exotoxin (CET) is a 634 amino acid residue protein whose sequence is set forth as in FIG. 9C and in SEQ ID NO:1. For convenience of reference, the terms “cholera exotoxin,” and “CET” as used herein may refer to the native or mature toxin, but more commonly refer to forms in which the toxin has been modified to reduce non-specific binding, for example, by deletion of domain Ia, or otherwise improve its utility for use in immunotoxins. Which meaning is intended will be clear in context.

A CET protein may be a full-length CET protein or it may be a partial CET protein comprising one or more subdomains of a CET protein and having cytotoxic activity as described herein. Mature CET has four domains: Domain Ia (amino acid residues 1-269, as shown in FIG. 9C and in SEQ ID NO:1), Domain II (amino acid residues 270-386, as shown in FIG. 9C and in SEQ ID NO:1), Domain Ib (amino acid residues 387-415, as shown in FIG. 9C and in SEQ ID NO:1), and Domain III (amino acid residues 417-634, as shown in FIG. 9C and in SEQ ID NO:1). The term “CET” includes a polypeptide, a polymorphic variant, an allele, a mutant of a CET that has cytotoxic activity and further (i) has an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 100, 150, 200, 250, 300, 500 or more amino acids, to a CET selected from a CET having an amino acid sequence of SEQ ID NO:1 or SEQ ID NO:2; (ii) comprises a furin cleavage sequence, an NAD binding site, and a KDEL (SEQ ID NO:4) motif as shown in FIG. 9B; (iii) binds to antibodies, e.g., polyclonal antibodies raised against an immunogen comprising an amino acid sequence of SEQ ID NO:1; and/or (iv) has ADP-ribosyltransferase activity as described herein.

A “CET nucleic acid” or “CET polynucleotide” refers to a gene encoding a CET protein. A “CET nucleic acid” includes both naturally occurring, recombinant and synthetic forms. A CET polynucleotide or CET polypeptide encoding sequence is typically from a bacterial pathogen, such as Vibrio cholerae. A CET polynucleotide may be a full-length CET polynucleotide, i.e., encoding a full-length CET protein or it may be a partial CET polynucleotide encoding a partial CET protein, such as a CET protein having one or more subdomains of a CET protein. A CET nucleic acid specifically hybridize under stringent hybridization conditions to a nucleic acid sequence having SEQ ID NO:3, SEQ ID NO:33, or conservatively modified variants thereof or has a nucleic acid sequence that has greater than about 90%, preferably greater than about 96%, 97%, 98%, 99%, or higher nucleotide sequence identity, preferably over a region of at least about 30, 50, 100, 200, 500, 750, 1000, 1250, 1,500, or more nucleotides, to SEQ ID NO:3 or SEQ ID NO:33.

As used herein, the term “conservative substitution”, when describing a protein refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups in Table A each contain amino acids that are conservative substitutions for one another:

TABLE A

1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

See also, Creighton, Proteins: Structures and Molecular Properties, W.H. Freeman and Company, New York (2nd Ed., 1992). Substituting in any protein any one of amino acid residue within one group by another amino acid residue of the same group will result in a conservative substitution and the resulting protein may be referred to as a conservatively modified derivative or fragment of that protein.

As used herein, the term “contacting” includes reference to placement in direct physical association.

As used herein, the term e term “cytotoxic agent” as used herein refers to a substance that inhibits or prevents the function of cells and/or causes destruction of cells. The term is intended to include toxins such as enzymatically active toxins of bacterial, fungal, plant or animal origin, or cytotoxic fragments thereof.

As used herein, the term “diphtheria toxin” or “DT” refers to a protein secreted by toxigenic strains of Corynebacterum diphtheriae. It is initially synthesized as a 535 amino acid polypeptide which undergoes proteolysis to form the toxin, which is composed of two subunits, A and B, joined by a disulfide bond. The B subunit, found at the carboxyl end, is responsible for cell surface binding and translocation; the A subunit, which is present on the amino end, is the catalytic domain, and causes the ADP ribosylation of Elongation Factor 2 (“EF-2”), thereby inactivating EF-2. See generally, Uchida et al., Science 175:901-903 (1972); Uchida et al., J Biol Chem 248:3838-3844 (1973). Mutated forms of DT suitable for use in immunotoxins are known in the art. See, e.g., U.S. Pat. Nos. 5,208,021 and 5,352,447. Once again, for convenience of reference, the term “DT” as used herein refers to the native toxin, but more commonly is used to refer to forms in which the B subunit has been deleted and in which modifications have been made in the A subunit to reduce non-specific binding and toxicity.

As used herein, the terms “effective amount” or “amount effective to” or “therapeutically effective amount” include reference to a dosage of a therapeutic agent sufficient to produce a desired result, such as inhibiting cell protein synthesis by at least 50%, or killing the cell.

As used herein, the term “effector moiety” means the portion of an immunoconjugate intended to have an effect on a cell targeted by the targeting moiety or to identify the presence of the immunoconjugate. Thus, the effector moiety can be, for example, a therapeutic moiety, a toxin, a radiolabel, or a fluorescent label. In the case of the present invention, the effector moiety is cholix toxin.

As used herein, the term “encoding” with respect to a specified nucleic acid, includes reference to nucleic acids which comprise the information for translation into the specified protein. The information is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Proc. Nat'l Acad. Sci. USA, 82:2306-2309 (1985)), or the ciliate Macronucleus, may be used when the nucleic acid is expressed in using the translational machinery of these organisms.

As used herein, the term “expressed” includes reference to translation of a nucleic acid into a protein. Proteins may be expressed and remain intracellular, become a component of the cell surface membrane or be secreted into the extracellular matrix or medium.

As used herein, the term “expression plasmid” comprises a nucleotide sequence encoding a molecule or interest, which is operably linked to a promoter.

As used herein, the term “fusing in frame” or grammatical equivalents thereof refer to joining two or more nucleic acid sequences which encode polypeptides so that the joined nucleic acid sequence translates into a single chain protein which comprises the original polypeptide chains.

As used herein, the term “host cell” refers to a cell which can support the replication or expression of the expression vector. Host cells may be prokaryotic cells such as E. coli, or eukaryotic cells such as yeast, insect, amphibian, or mammalian cells.

As used herein, the terms “identical” or “percent identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

As used herein, the term “substantially identical,” in the context of two nucleic acids or polypeptides, refers to two or more sequences or subsequences that have at least 60%, more preferably 65%, even more preferably 70%, still more preferably 75%, even more preferably 80%, and most preferably 90%, 91%, 92%, 93%, 94%, 95%, 96%, 97%, 98%, 99%, or higher nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math., 2:482 (1981), by the homology alignment algorithm of Needleman & Wunsch, J Mol. Biol., 48:443 (1970), by the search for similarity method of Pearson & Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al., J. Mol. Biol., 215:403-410 (1990) and Altschuel et al. Nucleic Acids Res., 25:3389-3402 (1977), respectively. Software for performing BLAST analyses is publicly available through the National Center for Biotechnology Infoiuiation (available on the internet by entering “http://www.ncbi.” followed by “nlm.nih.gov/”). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA, 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

As used herein, the term “immunoconjugate” includes reference to a covalent linkage of an effector molecule to an antibody. The effector molecule can be a toxin.

As used herein, the term “immunologically reactive condition” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See Harlow & Lane, supra, for a description of immunoassay foimats and conditions. Preferably, the immunologically reactive conditions employed in the methods of the present invention are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

As used herein, the terms “inhibiting the growth of a cell, “inhibiting the growth of a population of cells” or grammatical equivalents thereof refer to inhibiting cell division and may include destruction of the cell. The term also refers to any inhibition in cell growth and proliferation characteristics in vitro or in vivo of a cell, preferably a cancer cell, such as inhibiting formation of foci, inhibiting anchorage independence, inhibiting semi-solid or soft agar growth, inhibiting loss of growth factor or serum requirements, inhibiting changes in cell morphology, inhibiting immortalization, inhibiting expression of tumor specific markers, and/or inhibiting formation of tumors of the cell. See, e.g., Freshney, Culture of Animal Cells a Manual of Basic Technique pp. 231-241 (3rd ed. 1994).

As used herein, the terms “in vitro” and “ex vivo” means outside the body of the organism from which the cell was obtained.

As used herein, the “in vivo” includes reference to inside the body of the organism from which the cell was obtained.

The terms “isolated,” “purified,” or “biologically pure” refer to material, such as a PE, Ct, or CET as described herein, that is substantially or essentially free from components that normally accompany it as found in its native state or when made recombinantly. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “isolated” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

As used herein, the term “ligand” refers generically to molecules which bind specifically to a receptor or antigen on a cell surface. In preferred forms, the term encompasses both cytokines and antibodies or fragments thereof which retain recognition and binding capability for the antigen. In the most preferred form, the term refers to antibodies or fragments thereof which retain antigen recognition and binding capability. A variety of agents, such as cytokines, are known to bind to specific receptors on cell surfaces and can be used to targeted toxins to cells bearing such receptors. For example, IL-13 has been used as an agent to target toxins to cells over-expressing the IL-13 receptor. Antibodies bind specific antigens and are another type of agent used to direct toxins to desired target cells.

As used herein, the term “malignant cell” or “malignancy” refers to tumors or tumor cells that are invasive and/or able to undergo metastasis, i.e., a cancerous cell.

As used herein, the term “mammalian cell” includes reference to a cell derived from a mammal including humans, rats, mice, guinea pigs, chimpanzees, or macaques. The cell may be cultured in vivo or in vitro.

As used herein, the term “not the same” means different, not of the same identity.

As used herein, the term “nucleic acid” or “nucleic acid sequence” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof as well as conservative variants, i.e., nucleic acids present in wobble positions of codons and variants that, when translated into a protein, result in a conservative substitution of an amino acid.

As used herein, the terms, “polypeptide”, “peptide” and “protein” are used interchangeably and include reference to a polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

As used herein, the term “population of cells” refers to cells, preferably mammalian cells, grown in vitro or in vivo.

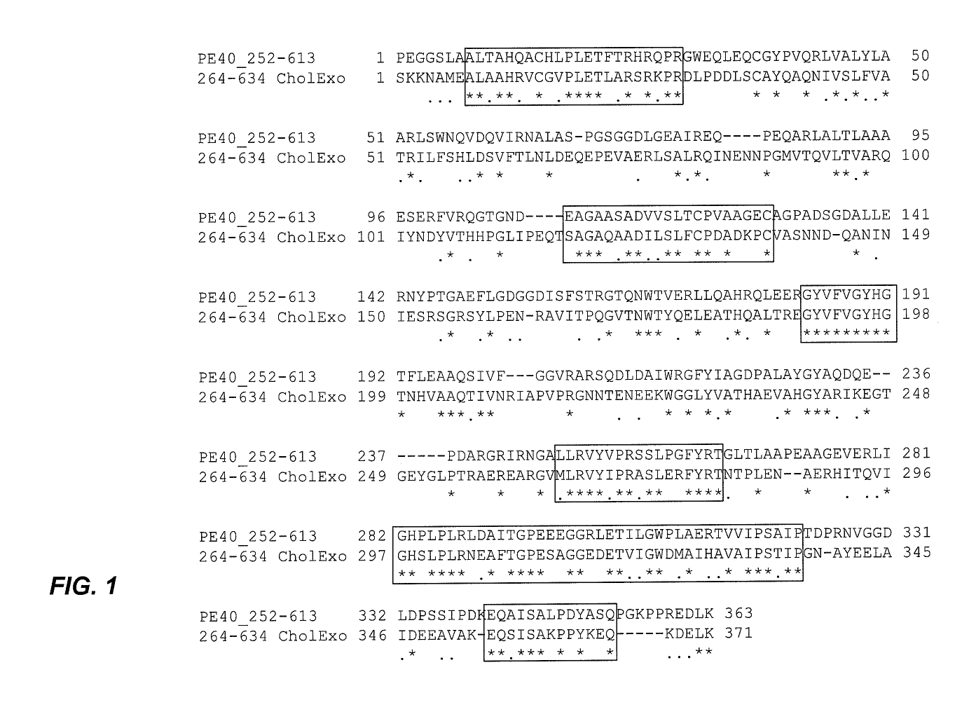

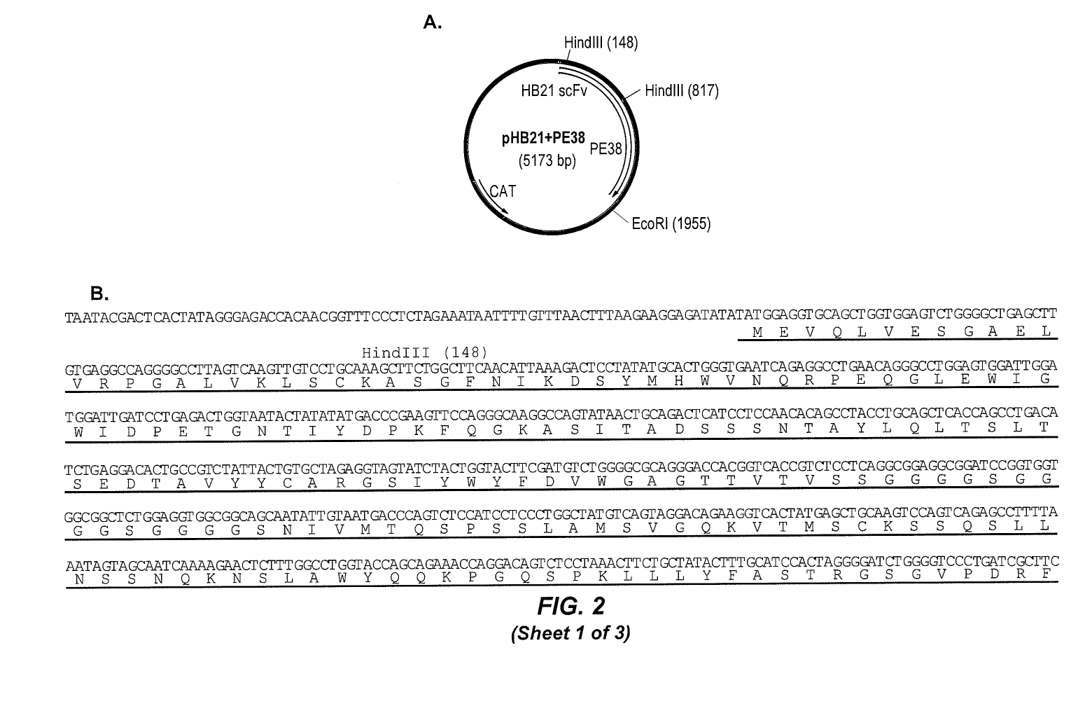

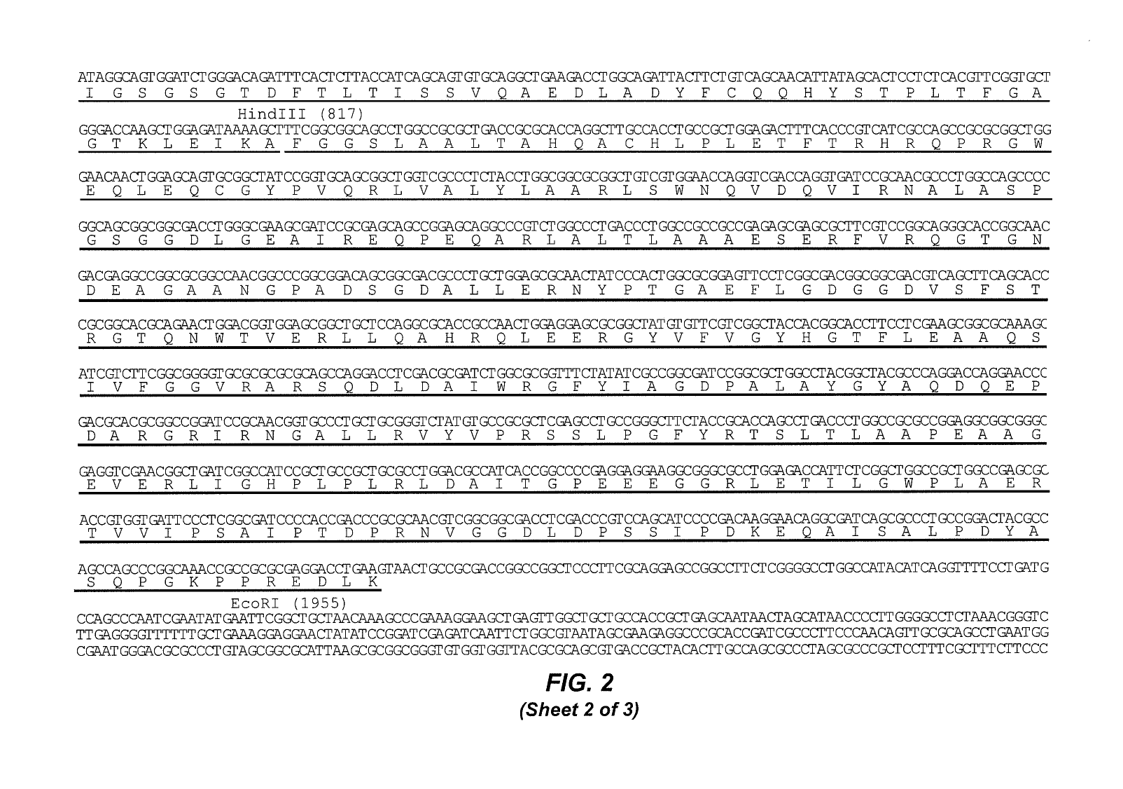

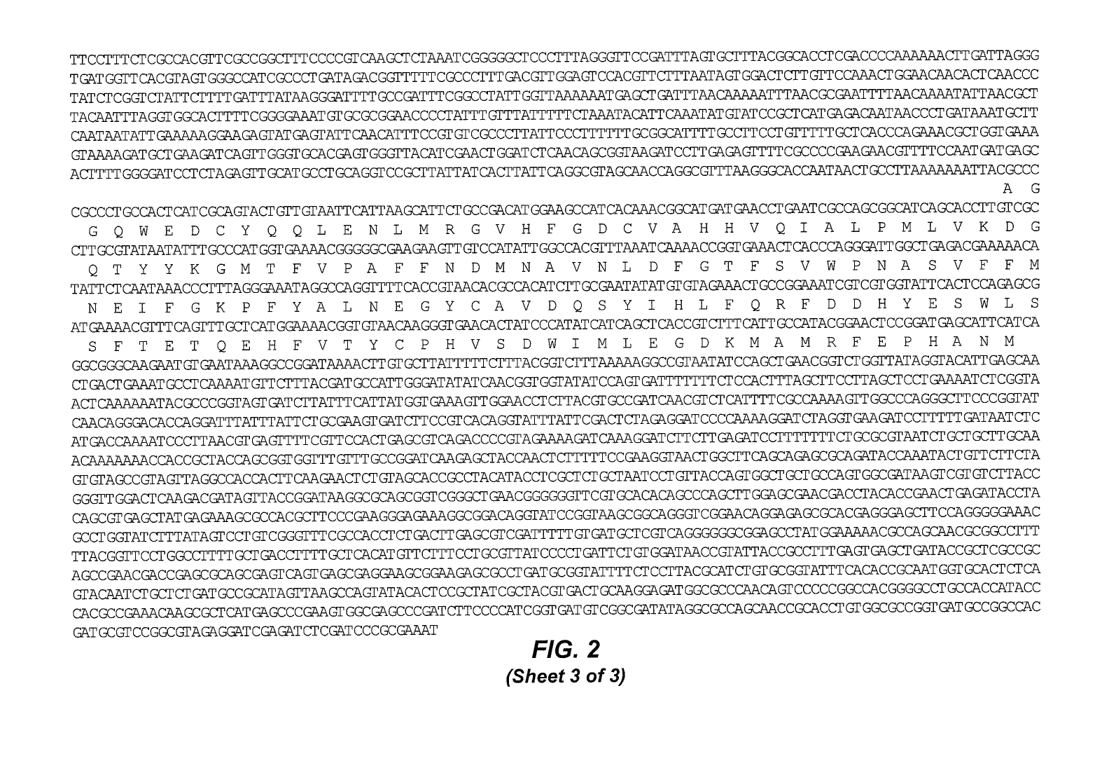

As used herein, the terms “Pseudomonas exotoxin A,” “Pseudomonas exotoxin” or “PE” refers to an extremely active monomeric protein (molecular weight 66 kD), secreted by Pseudomonas aeruginosa, which inhibits protein synthesis in eukaryotic cells. The 613-residue sequence of PE is well known in the art and is set forth, for example, in SEQ ID NO:1 of U.S. Pat. No. 5,602,095. The method of action and structure of PE as well as the modifications resulting in a number of variants of PE, are well known in the art. Mutations of PE are typically described in the art by reference to the amino acid residue present at a given position in the 613-amino acid sequence of native PE, even if the particular PE has been truncated to contain less than 613 residues. Thus, for example, the term “R490A” would indicate that the “R” (arginine, in standard single letter code) residue at position 490 in native PE has been replaced by an “A” (alanine, in standard single letter code) in the mutated PE under discussion. For convenience of reference, the term “Pseudomonas exotoxin A” and “PE”, as used herein, may refer to the native toxin, but more commonly refer to forms in which the toxin has been modified to reduce non-specific binding, for example, by deletion of domain Ia, or otherwise improve its utility for use in immunotoxins. Which meaning is intended will be clear in context. “PE” as used herein, also includes a PE that has been modified from the native protein to reduce binding and uptake via LRP1/CD91 (the cell surface receptor bound by the full-length toxin), to eliminate folding problems, or to reduce non-specific toxicity. Numerous such modifications are known in the art and include, elimination of domain Ia, various amino acid deletions in domains Ib, II and III, single amino acid substitutions and the addition of one or more sequences at the carboxyl terminus. See, e.g., Siegall et al., J Biol. Chem., 264:14256-14261 (1989). Cytotoxic fragments of PE include those which are cytotoxic with or without subsequent proteolytic or other processing in the target cell (e.g., as a protein or pre-protein). The term “PE” includes a polypeptide, a polymorphic variant, an allele, a mutant of a PE that has cytotoxic activity and further (i) has an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 100, 150, 200, 250, 300, 350, or more amino acids, to a PE selected from the PE having SEQ ID NO:13 (FIG. 1) and SEQ ID NO:25 (FIG. 9B); (ii) comprises a furin cleavage sequence, an NAD binding site and a RDEL (SEQ ID NO:7) motif as shown in FIG. 9B; (iii) binds to antibodies, e.g., polyclonal antibodies raised against an immunogen comprising an amino acid sequence of SEQ ID NO:13 or SEQ ID NO:25; and/or (iv) has ADP-ribosyltransferase activity as described herein.

A “PE nucleic acid” or “PE polynucleotide” refers to a gene encoding a PE protein.

A “PE nucleic acid” includes both naturally occurring, recombinant and synthetic forms. A PE polynucleotide or PE polypeptide encoding sequence is typically from a bacterial pathogen, such as Pseudomonas aeruginosa. A PE polynucleotide may be a full-length PE polynucleotide, i.e., encoding a complete PE protein or it may be a partial PE polynucleotide encoding a partial PE protein, such as a PE protein having one or more subdomains of a PE protein. A PE nucleic acid specifically hybridize under stringent hybridization conditions to the nucleic acid sequence of SEQ ID NO:15 encoding PE38 or to conservatively modified variants thereof or has a nucleic acid sequence that has greater than about 90%, preferably greater than about 96%, 97%, 98%, 99%, or higher nucleotide sequence identity, preferably over a region of at least about 30, 50, 100, 200, 500, 750, 1000, or more nucleotides, to the nucleic acid sequence of SEQ ID NO:15 encoding PE38.

As used herein, the term “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) faun of the cell, express mutants of genes that are found within the native form, or express native genes that are otherwise abnormally expressed, underexpressed or not expressed at all.

As used herein, the term “selectively reactive” refers, with respect to an antigen, the preferential association of an antibody, in whole or part, with a cell or tissue bearing that antigen and not to cells or tissues lacking that antigen. It is, of course, recognized that a certain degree of non-specific interaction may occur between a molecule and a non-target cell or tissue. Nevertheless, selective reactivity, may be distinguished as mediated through specific recognition of the antigen. Although selectively reactive antibodies bind antigen, they may do so with low affinity. On the other hand, specific binding results in a much stronger association between the antibody and cells bearing the antigen than between the bound antibody and cells lacking the antigen. Specific binding typically results in greater than 2-fold, preferably greater than 5-fold, more preferably greater than 10-fold and most preferably greater than 100-fold increase in amount of bound antibody (per unit time) to a cell or tissue bearing a target antigen as compared to a cell or tissue lacking the target antigen. Specific binding to a protein under such conditions requires an antibody that is selected for its specificity for a particular protein. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, ANTIBODIES, A LABORATORY MANUAL, Cold Spring Harbor Publications, New York (1988), for a description of immunoassay formats and conditions that can be used to determine specific immunoreactivity.

As used herein, the term “specifically (or selectively) binds” or “specifically (or selectively) immunoreactive with,” when referring to the binding of an antibody or antibody fragment to a cell surface marker, protein or peptide, refers to a binding reaction that is determinative of the presence of the cell surface marker or protein in a heterogeneous population of cell surface markers or proteins and, typically, other biologics. Thus, under designated conditions, a specified antibody or antibody fragment binds to a particular cell surface marker or protein at least two times the background and does not substantially bind in a significant amount to other cell surface markers or proteins present in a sample. Specific binding to an antibody or antibody fragment under such conditions may require an antibody or antibody fragment that is selected for its specificity for a particular cell surface marker or protein. For example, polyclonal antibodies raised against CET, as shown herein, or splice variants, or portions thereof, can be selected to obtain only those polyclonal antibodies that are specifically immunoreactive with CET and not with other proteins. This selection may be achieved by subtracting out antibodies that cross-react with molecules other than CET. In addition, polyclonal antibodies raised to CET polymorphic variants, alleles, orthologs, and conservatively modified variants can be selected to obtain only those antibodies that recognize CET, but not other CET subfamily members. Typically a specific or selective reaction will be at least twice background signal or noise and more typically more than 10 to 100 times background. The term also refers to binding that is saturable or competable by excess of the same antibody.

The phrase “selectively (or specifically) hybridizes to” or grammatical equivalents thereof refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, e.g., when a copy of a nucleic acid, such as a synthetic nucleic acid, is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel et al.

As used herein, the term “substantially similar” in the context of a peptide indicates that a peptide comprises a sequence with at least 90%, preferably at least 95% sequence identity to the reference sequence over a comparison window of 10-20 amino acids. Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the term “targeting moiety” refers to the portion of a targeted toxin intended to target the toxin to a cell of interest. Typically, the targeting moiety is an antibody, or a fragment of an antibody that retains antigen recognition capability, such as a scFv, a dsFv, an Fab, or an F(ab′)₂, but it can also be, for example, a cytokine (e.g., IL-13), or other protein (such as transferrin) that binds a specific receptor, preferably a captor on a cell surface.

As used herein, the term “targeted toxin” refers to a toxin which is covalently linked to, and targeted to desired cells by, a ligand which binds to specific receptors or antigens present on the surface of such cells. The term “immunotoxin” refers to a targeted toxin in which the toxin is targeted to the desired cells by an antibody or fragment thereof which retains antigen recognition and binding capability.

As used herein, the term “therapeutic agent” includes any number of compounds currently known or later developed to act as anti-neoplastics, anti-inflammatories, cytokines, anti-infectives, enzyme activators or inhibitors, allosteric modifiers, antibiotics or other agents administered to induce a desired therapeutic effect in a patient. The therapeutic agent may also be a toxin or a radioisotope, where the therapeutic effect intended is, for example, the killing of a cancer cell.

As used herein, the term “therapeutic moiety” refers to the portion of an targeted toxin intended to act as a therapeutic agent.

As used herein, the term “toxic moiety” refers to the portion of a targeted toxin which renders the targeted toxin cytotoxic to a cell of interest.

As used herein, the term “toxin” refers to a protein that is cytotoxic for a cell at concentrations, typically below one micromolar.

“Transforming growth factor α ” or “TGFα” is a well known growth factor which in its mature faun is a 5.5 kD, 50 amino acid protein. See, e.g., Brown, “The epidermal growth factor/transforming growth factor-α family and their receptors”. Eur J Gastroenterol Hepatol 7:914-922 (1995); Soler C., and Carpenter G., Thomson A. W., ed. “The epidermal growth factor (EGF) family”. The Cytokine Handbook, 3rd ed., San Diego, Calif., (pages 194-197 (1998). Recombinant human TGF α is commercially available from, for example, Sigma-Aldrich (catalog no. T7924, Sigma-Aldrich Corp., St. Louis, Mo.).

### II. Compositions

A. Bacterial Protein Toxins

Compositions of the present invention are typically provided as isolated or purified compositions.

1. Pseudomans Exotoxin A

Pseudomonas exotoxin A (PE) contains three structural domains that act in concert to cause cytotoxicity. The 613 amino acid sequence of PE is set forth as SEQ ID NO:1 of U.S. Pat. No. 5,602,095. Domain Ia (amino acids 1-252) mediates cell binding. Domain II (amino acids 253-364) is responsible for translocation into the cytosol and domain III (amino acids 400-613) mediates ADP ribosylation of elongation factor 2. The function of domain Ib (amino acids 365-399) remains undefined, although it has been known a large part of it, amino acids 365-380, can be deleted without loss of cytotoxicity. See Siegall et al., J Biol Chem, 264:14256-61 (1989).

Certain cytotoxic fragments of PE are known in the art and are often referenced by the molecular weight of the fragment, which designates for the person of skill in the art the particular composition of the PE fragment. For example, PE40 was one of the first fragments that was studied and used as the toxic portion of immunotoxins. The term designates a truncated form of PE in which domain Ia, the domain responsible for non-specific binding. See, e.g., Pai et al., Proc. Nat'l Acad. Sci. USA, 88:3358-3362 (1991); and Kondo et al., J. Biol. Chem., 263:9470-9475 (1988). Elimination of non-specific binding, however, can also be achieved by mutating certain residues of domain Ia. U.S. Pat. No. 5,512,658, for instance, discloses that a mutated PE in which domain Ia is present but in which the basic residues of domain Ia at positions 57, 246, 247, and 249 are replaced with acidic residues (glutamic acid, or “E”)) exhibits greatly diminished non-specific cytotoxicity. This mutant form of PE is sometimes referred to as “PE4E.”

A preferred PE40 of the present invention comprises an amino acid sequence of SEQ ID NO:13 or a conservatively modified derivative thereof. Another preferred PE40 of the present invention comprises an amino acid sequence of SEQ ID NO:25 or a conservatively modified derivative thereof. Yet another preferred PE40 of the present invention consists of an amino acid sequence of SEQ ID NO:13 or a conservatively modified derivative thereof. Another preferred PE40 of the present invention consists of an amino acid sequence of SEQ ID NO:25 or a conservatively modified derivative thereof.

“PE38” refers to a cytotoxic fragment of PE having a molecular weight of approximately 38 kD. It contains the translocating and ADP ribosylating domains of PE but not the cell-binding portion (Hwang J. et al., Cell, 48:129-136 (1987)). PE38 is a truncated PE pro-protein composed of amino acids 253-364 and 381-613 which is activated to its cytotoxic faun upon processing within a cell (see, e.g., U.S. Pat. No. 5,608,039, and Pastan et al., Biochim. Biophys. Acta, 1333:C1-C6 (1997)).

A preferred PE38 comprises an amino acid sequence shown in SEQ ID NO:30 or a conservatively modified derivative thereof. Another preferred PE38 comprises an amino acid sequence which is shown as part of SEQ ID NO:16 in the context of a P38 immunotoxin or a conservatively modified derivative thereof. Yet another preferred PE38 consists of an amino acid sequence shown in SEQ ID NO:30 or a conservatively modified derivative thereof. Another preferred PE38 consists of an amino acid sequence which is shown as part of SEQ ID NO:16 in the context of a P38 immunotoxin or a conservatively modified derivative thereof.

The sequence of PE38 is well known in the art, but can also readily be determined by the practitioner by subtracting the stated residues from the known sequence of PE. Persons of skill will be aware that, due to the degeneracy of the genetic code, the amino acid sequence of PE38, of its variants, such as PE38 KDEL or PE38QQR, and of the other PE derivatives discussed herein can be encoded by a great variety of nucleic acid sequences, any of which can be expressed to result in the desired polypeptide.

“PE35” is a 35 kD carboxyl-terminal fragment of PE in which amino acid residues 1-279 have deleted and the molecule commences with a methionine at position 280 followed by amino acids 281-364 and 381-613 of native PE. PE35 and PE40 are disclosed, for example, in U.S. Pat. Nos. 5,602,095 and 4,892,827.

A preferred PE35 comprises an amino acid sequence shown in SEQ ID NO:32 or a conservatively modified derivative thereof. Another preferred PE35 consists of an amino acid sequence shown in SEQ ID NO:32 or a conservatively modified derivative thereof.

Studies also determined that mutations of the terminal residues of PE, REDLK (SEQ ID NO:5, residues 609-613) could be varied in ways that would increase the cytotoxicity of the resulting mutant. For example, immunotoxins made with mutated PEs ending in the sequences KDEL (SEQ ID NO:4), REEL (SEQ ID NO:6) or RDEL (SEQ ID NO:7) were much more cytotoxic to target cells than were like immunotoxins made with PE38 bearing the native terminal sequence. See, Kreitman and Pastan, Biochem J, 307(Pt 1):29-37 (1995). Repeats of these sequences can also be used. See, e.g., U.S. Pat. Nos. 5,854,044; 5,821,238; and 5,602,095 and International Publication WO 99/51643.

2. Cholix Toxin (CT)

Mature cholix toxin (CT) is a 70.7 kD, 634 residue protein, whose sequence is set forth as SEQ ID NO:31 and FIG. 9C. The sequence, with an eight residue leader sequence consisting of a 6-histidine tag flanked by a methionine on each side (SEQ ID NO:20), is publicly available on-line in the Entrez Protein database under accession number 2Q5T_A.

3. Cholera Exotoxin (CET)

Mature cholera exotoxin is a 634 residue protein, whose sequence is set forth as SEQ ID NO:1 and in FIG. 9C.

In one embodiment of the present invention, a CET comprises an amino acid sequence of SEQ ID NO:1 or a conservatively modified derivative thereof.

As shown in FIG. 9C, the amino acid sequence of CET differs from that of cholix toxin in the following 14 amino acid positions: 90, (CT=H; CET=N), 213 (CT=M; CET=I), 245 (CT=V; CET=A), 266 (CT=G; CET=K), 270 (CT=S; CET=E), 295 (CT=T; CET=P), 342 (CT=D, CET=A), 345 (CT=R, CET=Q), 376 (CT=T, CET=I), 400 (CT=S; CET=P), 523, (CT=D; CET=E), 553 (CT=E; CET=R), 622 (CT=T; CET=A), and 629 (CT=R; CET=Q).

In another embodiment of the present invention, a CET comprises an amino acid sequence of SEQ ID NOS:1, 2, 24, or a conservatively modified derivative thereof and having at least one the following amino acid residues with respect to SEQ ID NO:2: 90N, 2131, 245A, 266K, 270E, 295P, 342A, 345Q, 3761, 400P, 523E, 553R, 622A, or 629Q.

4. Modifications Of Cholix Toxin

A CT underlying the present invention comprises or consists of a truncated version of CT in which the receptor binding domain, domain Ia, is deleted, to create a 40 kD version of CT corresponding to PE40 and referred to herein as “CT40.” A preferred CT40 protein of the present invention is set forth in FIG. 9B and is a CT40 protein having SEQ ID NO:23. Given the similarity of CT and PE (see FIG. 9B), it is expected that additional variants of CT, such as a CT38 or CT35 variant, can be made that correspond to variants of PE as described in the preceding section. For example, it is anticipated that some or all of CT domain Ib can be deleted which, with the deletion of domain Ia, would create a CT variant akin to PE38. Similarly, it is anticipated that the carboxyl terminus of CT, which ends with KDELK (SEQ ID NO:8) (see FIG. 9B), can be varied by replacing it with one of the various C-terminal sequences mentioned above as maintaining the toxicity of PE. In preferred embodiments, if the C-terminal sequence of CT is replaced, the C-terminal sequence used as a replacement is one suitable for use in humans. In some preferred embodiments, the C-terminal sequence of CT (KDELK, SEQ ID NO:8) is replaced with the terminal sequence of PE, REDLK (SEQ ID NO:5).

Similarly, it is anticipated that the NAD domain of CT, which at least comprises amino acid residues GGEDETVIG (SEQ ID NO:28; see FIG. 9B) can be varied by replacing it with another NAD domain sequence. In preferred embodiments, if the NAD domain sequence of CT is replaced, the NAD domain sequence used as a replacement is one suitable for use in humans. In some preferred embodiments, the NAD domain sequence of CT (GGEDETVIG (SEQ ID NO:28) is replaced with the NAD binding site of PE comprising the amino acid sequence GGRLETILG (SEQ ID NO:30).

In a preferred embodiment, a cytotoxic fragment of CT retains at least about 10%, preferably at least about 40%, more preferably about 50%, even more preferably 75%, more preferably at least about 90%, and still more preferably 91%, 92%, 93%, 94%, 95%, 96%, 97%, 98%, 99% or more of the cytotoxicity of CT. In particularly preferred embodiments, the cytotoxic fragment has at least the cytotoxicity of CT40, and preferably has more. CTs employed in the invention can be assayed for the desired level of cytotoxicity by assays well known to those of skill in the art. For example, any particular fragment CT and conservatively modified variants of such fragments can be readily assayed for cytotoxicity by the assays used in the studies underlying the present invention, as described at the beginning of the Detailed Description.

5. Modifications of Cholera Exotoxin

The CET used in the studies underlying the present invention consisted of a truncated version of CET in which the receptor binding domain, domain Ia, was deleted, to create a 40 kD version of CET corresponding to PE40, referred to herein as “CET40.”

In a preferred embodiment, a CET is a CET40. A preferred CET40 protein of the present invention is set forth in FIG. 9B and is a CET40 protein having SEQ ID NO:24. Thus, in a preferred embodiment of the present invention a CET comprises an amino acid sequence of SEQ ID NO:24 or a conservatively modified derivative thereof. In another preferred embodiment of the present invention, a CET comprises an amino acid sequence of SEQ ID NO:2 or a conservatively modified derivative thereof. In yet other preferred embodiment of the present invention a CET consists of an amino acid sequence of SEQ ID NO:24 or a conservatively modified derivative thereof. In another preferred embodiment of the present invention, a CET consists of an amino acid sequence of SEQ ID NO:2 or a conservatively modified derivative thereof.

In another embodiment of the present invention, a CET40 protein is encoded by a CET nucleic acid. A preferred CET nucleic acid encoding a CET40 protein is a nucleic acid having SEQ ID NO:3. In SEQ ID NO:3, the coding region of CET40 is preceded by a short linker sequence comprising a HindIII restriction site. In addition, immediately following the nucleotides coding for the C-terminal amino acid residues of CET40 (KDELK; SEQ ID NO:8), two in frame stop codons are present. Another preferred CET nucleic acid is a nucleic acid having SEQ ID NO:33. Both, SEQ ID NOS:3 and 33 are non-naturally occurring, synthetic nucleic acids.

Given the similarity of CET and PE (see FIG. 9B), it is expected that additional variants of CE such as a CET38 or CET35 variant, can be made that correspond to variants of PE as described in the preceding section. For example, it is anticipated that some or all of CET domain lb can be deleted which, with the deletion of domain Ia, would create a CET variant akin to PE38. Similarly, it is anticipated that the carboxyl terminus of CET, which ends with KDELK (SEQ ID NO:8) (see FIG. 9B), can be varied by replacing it with one of the various C-terminal sequences mentioned above as maintaining the toxicity of PE. In preferred embodiments, if the C-terminal sequence of CET is replaced, the C-terminal sequence used as a replacement is one suitable for use in humans. In some preferred embodiments, the C-terminal sequence of CET (KDELK, SEQ ID NO:8) is replaced with the terminal sequence of PE, REDLK (SEQ ID NO:5).

Similarly, it is anticipated that the NAD domain of CET, which comprises at least amino acid residues GGEDETVIG (SEQ ID NO:28) (see FIG. 9B) can be varied by replacing it with another NAD domain sequence. In preferred embodiments, if the NAD domain sequence of CET is replaced, the NAD domain sequence used as a replacement is one suitable for use in humans. In some preferred embodiments, the NAD domain sequence of CET (GGEDETVIG (SEQ ID NO:28) is replaced with the NAD binding site of PE comprising the amino acid sequence GGRLETILG (SEQ ID NO:29).

In a preferred embodiment, a cytotoxic fragment of CET retains at least about 10%, preferably at least about 40%, more preferably about 50%, even more preferably 75%, more preferably at least about 90%, and still more preferably 91%, 92%, 93%, 94%, 95%, 96%, 97%, 98%, 99% or more of the cytotoxicity of CET. In particularly preferred embodiments, the cytotoxic fragment has at least the cytotoxicity of CET40, and preferably has more. CETs employed in the invention can be assayed for the desired level of cytotoxicity by assays well known to those of skill in the art. For example, any particular fragment CET and conservatively modified variants of such fragments can be readily assayed for cytotoxicity by the assays used in the studies underlying the present invention, as described at the beginning of the Detailed Description.

6. Isolated Toxins and Chimeric Toxin Proteins

In one aspect of the present invention, an isolated toxin is a chimeric toxin protein. In preferred embodiments of the present invention a chimeric toxin protein is an immunotoxin.

In a preferred embodiment, the chimeric protein comprises (i) a toxin or a cytotoxic fragment thereof and (ii) a targeting moiety which specifically binds to at least one surface marker on a cell, preferably a targeting moiety which specifically binds to at least one surface marker of on a mammalian cell, and more preferably a targeting moiety which specifically binds to at least one surface marker on a human cell.

Preferably the human cell is a disease cell or a malignant cell and more preferably, the human cell is a cancer cell.

Preferably, an isolated toxin or chimeric protein comprises (i) a toxin or cytotoxic fragment thereof selected from the group consisting of Pseudomonas exotoxin A (PE), cholix toxin (CT), and cholera exotoxin (CET). The effectiveness (e.g., cell killing activity, neutralization) of a cytotoxic fragment of PE, CT or CET can be tested by a practitioner without undue experimentation using methods described herein. Methods described herein for making PE- and CET-based chimeric proteins, such as PE- and CET-based immunotoxins, can be used to make CT-based chimeric proteins, such as CT-based immunotoxins and additional PE- and CET-based immunotoxins. Thus, using the methods described herein, PE, CT, CET and cytotoxic fragments thereof, such as PE40, CT40, CET40 can be used to construct potent and antigen-specific recombinant immunotoxins. Nucleic acids encoding these toxins and cytotoxic fragments thereof can be fused in frame with a nucleic acid encoding a targeting moiety, such as an antibody, antibody fragment or ligand.

a) CET-Based Isolated Toxins and Chimeric Toxin Proteins

In some embodiments an isolated toxin is a CET-based chimeric toxin protein. In a preferred embodiment, the chimeric protein comprises CET or a cytotoxic fragment thereof A preferred cytotoxic fragment of CET is CET40. Another preferred cytotoxic fragment of CET is CET38. Yet another preferred cytotoxic fragment of CET is CT35. CET38 and CET35 can me made as described herein in analogy to PE38 and PE35.

A preferred isolated toxin or preferred chimeric protein comprises a CET that has (i) cytotoxic activity and (ii) an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 100, 150, 200, 250, 300, 500 or more amino acids, to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 85% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 90% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 91% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 92% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 93% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 94% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 95% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 96% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 97% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 98% amino acid sequence identity over a region of at least about 100 amino acids to a CET selected from a CET having SEQ ID NO:1, SEQ ID NO:2, and SEQ ID NO:24.

A preferred isolated toxin or preferred chimeric protein comprises a CET that has (i) cytotoxic activity and (ii) an amino acid sequence that has greater than about 99% amino acid sequence identity over a region of at least about 100 amino acids to a CET selected from a CET having SEQ ID NO:1, SEQ ID NO:2, and SEQ ID NO:24.

Another preferred isolated toxin or preferred chimeric protein comprises a CET that has (i) cytotoxic activity and (ii) comprises a furin cleavage sequence, an NAD binding site and a KDEL (SEQ ID NO:4) motif.

Another preferred isolated toxin or preferred chimeric protein comprises a CET that has (i) cytotoxic activity and (ii) binds to an antibody, e.g., a monoclonal or polyclonal antibody raised against an immunogen comprising an amino acid sequence of SEQ ID NO:1, SEQ ID NO:2, and SEQ ID NO:24.

In another embodiment of the present invention, a preferred isolated toxin or a preferred chimeric protein comprises a CET that has (cytotoxic activity and (ii) at least one the following amino acid residues with respect to SEQ ID NO:1: 90N, 2131, 245A, 266K, 270E, 295P, 342A, 345Q, 376I, 400P, 523E, 553R, 622A, or 629Q.

In another embodiment of the present invention, a preferred isolated toxin or a preferred chimeric protein comprises a CET that has (cytotoxic activity and (ii) at least one the following amino acid residues with respect to SEQ ID NO:2: 25P, 72A, 75Q, 106I, 130P, 253E, 283R, 352A, or 359Q.

In another embodiment of the present invention, a preferred isolated toxin or a preferred chimeric protein comprises a CET that has (cytotoxic activity and (ii) at least one the following amino acid residues with respect to SEQ ID NO:24: 26P, 73A, 76Q, 107I, 131P, 254E, 284R, 353A, or 360Q.

Another preferred isolated toxin or preferred chimeric protein comprises a CET that has (i) cytotoxic activity and (ii) an amino acid sequence that has greater than about 85%, greater than about 90%, greater than about 91%, greater than about 92%, greater than about 93%, greater than about 94%, greater than about 95%, greater than about 96%, greater than about 97%, greater than about 98%, or greater than about 99% amino acid sequence identity over a region of at least about 100 amino acids to a CET selected from a CET having SEQ ID NO:1, SEQ ID NO:2, and SEQ ID NO:24, and further comprises (1) at least one of amino acid residues 90N, 2131, 245A, 266K, 270E, 295P, 342A, 345Q, 3761, 400P, 523E, 553R, 622A, or 629Q of SEQ ID NO:1; (2) at least one of amino acid residues 25P, 72A, 75Q, 106I, 130P, 253E, 283R, 352A, or 359Q of SEQ ID NO:2, or (3) at least one of amino acid residues 26P, 73A, 76Q, 107I, 131P, 254E, 284R, 353A, or 360Q of SEQ ID NO:24.

A preferred CET chimeric protein is a targeted CET protein comprising a targeting moiety. Suitable targeting moieties are described in detail herein. The targeting moiety is fused in frame with the CET either at the carboxy- or amino terminus of CET. Where the targeting moiety is an antibody or antibody fragment, the targeted CET protein is also referred to herein as an “immunotoxin” more specifically, as a “CET immunotoxin.”

A preferred CET-based immunotoxin of the present invention is an greater than about 99% immunotoxin comprising CET40 having an amino acid sequence of SEQ ID NO:24 (FIG. 9B). A preferred chimeric protein of the present invention comprises a CET40 or a cytotoxic fragment thereof fused in frame to the N-terminal or C-terminal end of an antibody, antibody fragment or ligand.

A preferred CET-based immunotoxin of the present invention is an immunotoxin having an amino acid sequence of SEQ ID NO:22 (FIG. 9A) and referred to herein as HB21scFv-CET40. A preferred CET-based immunotoxin of the present invention is an immunotoxin encoded by a nucleic acid having a nucleic acid sequence of SEQ ID NO:21 (FIG. 9A).

In another preferred embodiment of the present invention, a chimeric CET protein is a targeted toxin comprising a toxin comprising (i) a furin cleavage sequence having an amino-terminal sequence and a carboxy-terminal sequence, and (ii) domain III of CET having an amino-terminal sequence and a carboxy-terminal sequence. The carboxy-terminal sequence of the furin cleavage sequence may be fused to the amino-terminal sequence of the CET domain III. Alternatively, the carboxy-terminal sequence of the CET domain III may be fused to the amino-terminal sequence of the furin cleavage sequence.

Preferably, the CET domain III comprises amino acid residues 417-634 as shown in FIG. 9C and in SEQ ID NO:36. In another embodiment, the CET domain III consists of amino acid residues 417-634 as shown in FIG. 9C and in SEQ ID NO:36.

Also preferred are fragments of CET domain III or conservatively modified variants of CET domain III having similarity to amino acid residues 417-634 as shown in FIG. 9C and in SEQ ID NO:36.

A preferred isolated toxin or preferred chimeric protein, such as a targeted toxin, comprises a CET domain III that has an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 75, 100, 125, 150, 175, 200 or more amino acids, to amino acid residues 417-634 as shown in FIG. 9C and in SEQ ID NO:36.

A preferred isolated toxin or preferred chimeric protein comprises a CET domain III that has an amino acid sequence that has greater than about 85% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 90% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 91% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 92% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 93% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 94% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 95% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 96% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 97% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 98% amino acid sequence identity over a region of at least about 75 amino acids to amino acid residues 417-634 as shown in FIG. 9C and in SEQ ID NO:36.

A preferred isolated toxin or preferred chimeric protein comprises a CET domain III that has an amino acid sequence that has greater than about 99% amino acid sequence identity over a region of at least about 75 amino acids to amino acid residues 417-634 as shown in FIG. 9C and in SEQ ID NO:36.

In another embodiment of the present invention, an isolated toxin or chimeric protein comprises CET domain III that has an amino acid sequence that has greater than about 85%, greater than about 90%, greater than about 91%, greater than about 92%, greater than about 93%, greater than about 94%, greater than about 95%, greater than about 96%, greater than about 97%, greater than about 98%, or greater than about 99% amino acid sequence identity over a region of at least about 75 amino acids to amino acid residues 417-634 as shown in FIG. 9C and in SEQ ID NO:36 and further comprises at least one of amino acid residues 253E, 283R, 352A, or 359Q of SEQ ID NO:2.

In another embodiment of the present invention, an isolated toxin or chimeric protein comprises a CET that has (cytotoxic activity and (ii) at least one the following amino acid residues with respect to SEQ ID NO:2: 25P, 72A, 75Q, 106I, 130P, 253E, 283R, 352A, or 359Q.

In another embodiment of the present invention, an isolated toxin or chimeric protein comprises a CET that has (cytotoxic activity and (ii) at least one the following amino acid residues with respect to SEQ ID NO:24: 26P, 73A, 76Q, 107I, 131P, 254E, 284R, 353A, or 360Q.

b) CT-based Isolated Toxins and Chimeric Toxin Proteins

In some embodiments an isolated toxin is a CT-based chimeric toxin protein. In another preferred embodiment, the chimeric protein comprises CT or a cytotoxic fragment thereof. A preferred cytotoxic fragment of CT is CT40. Another preferred cytotoxic fragment of CT is CT38. Yet another preferred cytotoxic fragment of CT is CT35. CT38 and CT35 can me made as described herein in analogy to PE38 and PE35.

A preferred isolated toxin or preferred chimeric protein comprises a CT that has (i) cytotoxic activity and (ii) an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 100, 150, 200, 250, 300, 500 or more amino acids, to a CT having SEQ ID NO:23.

Another preferred isolated toxin or preferred chimeric protein comprises a CT that has (i) cytotoxic activity and (ii) comprises a furin cleavage sequence, an NAD binding site and a KDEL motif.

Another isolated toxin or preferred chimeric protein comprises a CT that has (i) cytotoxic activity and (ii) binds to an antibody, e.g., a monoclonal or polyclonal antibody raised against an immunogen comprising an amino acid sequence of SEQ ID NO:23.

A preferred CT chimeric protein is a targeted CT protein comprising a targeting moiety. The targeting moiety is fused in frame with the CT either at the carboxy- or amino terminus of CT. Where the targeting moiety is an antibody or antibody fragment, the targeted CT protein is also referred to herein as an “immunotoxin” more specifically, as a “CT immunotoxin.”

A preferred CT-based immunotoxin of the present invention is an immunotoxin comprising CT40 having an amino acid sequence of SEQ ID NO:23. A preferred chimeric protein of the present invention comprises a CT40 or a cytotoxic fragment thereof fused in frame to the N-terminal or C-terminal end of an antibody, antibody fragment or ligand.

c) PE-based Isolated Toxins and Chimeric Toxin Proteins

In another preferred embodiment, an isolated toxin or preferred chimeric protein comprises PE or a cytotoxic fragment thereof. A preferred isolated toxin or preferred cytotoxic fragment of PE is PE40. Another preferred isolated toxin or preferred cytotoxic fragment of PE is PE38. Yet another preferred isolated toxin or preferred cytotoxic fragment of PE is PE35. Amino acid sequences for PE38 and PE35 are described herein in SEQ ID NOS:30 and 32, respectively.

A preferred isolated toxin or preferred chimeric protein comprises a PE that has (i) cytotoxic activity and (ii) an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 100, 150, 200, 250, 300, 350, or more amino acids, to a PE selected from the PE having SEQ ID NO:13 (FIG. 1) or SEQ ID NO:25 (FIG. 9B)

Another preferred isolated toxin or preferred chimeric protein comprises a PE that has (i) cytotoxic activity and (ii) comprises a furin cleavage sequence, an NAD binding site and a KDEL motif.

Another preferred isolated toxin or preferred chimeric protein comprises a PE that has (i) cytotoxic activity and (ii) binds to an antibody, e.g., a monoclonal or polyclonal antibody raised against an immunogen comprising an amino acid sequence of SEQ ID NO:13 or SEQ ID 25.

A preferred PE chimeric protein is a targeted PE protein comprising a targeting moiety. The targeting moiety is fused in frame with the PE either at the carboxy- or amino terminus of PE. Where the targeting moiety is an antibody or antibody fragment, the targeted PE protein is also referred to herein as an “immunotoxin” more specifically, as a “PE immunotoxin.”

A preferred PE-based immunotoxin of the present invention is an immunotoxin comprising PE40 having an amino acid sequence of SEQ ID NO:25 (FIG. 9B). Another preferred PE-based immunotoxin of the present invention is an immunotoxin comprising PE38 having an amino acid sequence of SEQ ID NO:31. A preferred chimeric protein of the present invention comprises a PE40 or a cytotoxic fragment thereof fused in frame to the N-terminal or C-terminal end of any antibody, antibody fragment or ligand.

A preferred PE-based immunotoxin of the present invention is an immunotoxin having an amino acid sequence of SEQ ID NO:16, referred to herein as HB21scFv-PE38. A preferred CET-based immunotoxin of the present invention is an immunotoxin encoded by a nucleic acid having a nucleic acid sequence of SEQ ID NO:15.

Another preferred PE-based immunotoxin of the present invention is an immunotoxin having an amino acid sequence of SEQ ID NO:35, referred to herein as HB21scFv-PE40. A preferred CET-based immunotoxin of the present invention is an immunotoxin encoded by a nucleic acid having a nucleic acid sequence of SEQ ID NO:34.

d) Chimeric Toxin Proteins Having an Antibody as Targeting Moiety

In a preferred embodiment, the targeting moiety is an antibody, preferably an antibody specifically binding to a surface marker on a cell. A surface marker can be any cell surface receptor. A preferred cell surface marker is a transferrin receptor. Other preferred cell surface markers include, but are not limited to, EGF receptor, CD19, CD22, CD25, CD31, CD79, mesothelin, and cadherin.

In another preferred embodiment, the targeting moiety is an antibody fragment, preferably an antibody fragment specifically binding to a surface marker on a cell. A preferred antibody fragment is a single chain Fv. Herein the construction and characterization of PE- and CET-based immunotoxins wherein the PE, and CET are fused to a scFv are described. CT-based immunotoxins can be made accordingly. Other preferred antibody fragments to which a toxin or cytotoxic fragment can be fused include Fab, Fab′, F(ab′)2, Fv fragment, a helix-stabilized antibody, a diabody, a disulfide stabilized antibody, and a domain antibody.

The fusion of a PE, CT, or CET to an antibody or antibody fragment can be either to the N-terminus or C-terminus of the antibody or antibody fragment. Such fusion typically is accomplished employing recombinant DNA technologies.

e) Chimeric Toxin Proteins Having a Ligand as Targeting Moiety

In another preferred embodiment, the targeting moiety is a ligand specifically binding to a receptor on a cell surface. The ligand can be any ligand which binds to a cell surface marker. A preferred ligand is VEGF, Fas, TRAIL, a cytokine, a hormone. Other preferred ligands include, but are not limited to, TGFα, IL-2, IL15, IL4.

7. Furin and Modifications to CT's and CET's Furin Cleavage Sequence

As reported by Duckert et al., Protein Engineering, Design & Selection 17(1):107-112 (2004) (hereafter, “Duckert et al.”), furin is an enzyme in a “family of evolutionarily conserved dibasic- and monobasic-specific CA²⁺-dependent serine proteases called substilisin/kexin-like proprotein convertases.” Id., at p. 107. Furin, also known as “paired basic amino acid cleaving enzyme” or “PACE”, is one of seven mammalian members of the family and is involved in processing several endogenous human proteins. See generally, e.g., Thomas G, Nat Rev Mol Cell Biol, (10):753-66 (2002). It is a membrane-associated protein found mainly in the trans-Golgi network. The sequence of human furin has been known since the early 1990s. See, e.g., Hatsuzawa, K. et al., J. Biol. Chem., 267:16094-16099 (1992); Molloy, S. et al., J. Biol. Chem., 267:16396-16402 (1992).

The minimal cleavage sequence for furin is, in the single letter code for amino acid residues, R-X-X-R (SEQ ID NO:9), with cleavage occurring after the second “R.” Duckert et al. summarized the information available on the sequences of 38 proteins reported in the literature to have furin cleavage sites, including mammalian proteins, proteins of pathogenic bacteria, and viral proteins. Duckert et al. reported that 31, or 81%, of the cleavage motifs reviewed had the R-X-[R/K]-R (SEQ ID NO:10) consensus sequence, of which 11, or 29%, had R-X-R-R (SEQ ID NO:11), and 20, or 52%, were R-X-K-R (SEQ ID NO:12). Three of the cleavage motifs contained only the minimal cleavage sequence. Duckert et al. further aligned the motifs and identified the residues found at each position in each furin both for the cleavage motif itself and in the surrounding residues. FIG. 1A of Duckert et al. shows by relative size the residues most commonly found at each position. By convention, the residues surrounding the furin cleavage site are numbered from the scissile bond (which is typically indicated by the symbol “↓”). Counting toward the N terminus, the substrate residues are designated P1, P2, and so on, while counting towards the C-terminus, the residues are designated P1′, P2′, and so on. See, e.g., Rockwell, N. C., and J. W. Thorner, Trends Biochem. Sci., 29:80-87 (2004); Thomas G., Nat. Rev. Mol. Cell. Biol., 3:753-766 (2002). Thus, following the convention, the following sequence can be used to align and number the residues of the minimal cleavage sequence and the surrounding residues:
P6-P5-P4-P3-P2-P1-P1′-P2′-P3′-P4′-P5′,
   in which the minimal furin cleavage sequence is numbered as P4-P1. Duckert et al.'s alignment of 38 sequences cleaved by furin identified the variations permitted depending on the residues present at various positions. For example, if the residue at P4 is not an R, that can be compensated for by having arginine or lysine residues at P2 and P6. Id., at p. 109.

In the case of CT and CT, the residues at positions P1′ and P2′ are D-L (see FIG. 9B), which are residues 293 and 294, respectively, of the native CT sequence, SEQ ID NO:31. PE has the residues G-W at positions P1′-P2′. The applicants have found that substitution of other residues for the tryptophan at position P2′ reduces cytotoxicity and that generally the presence of the glycine at position P1′ should be maintained. Accordingly, in some embodiments, the residues at positions P1′ and P2′ of the cholix toxins and immunotoxins of the invention are selected from the group consisting of G-W, D-W, and G-L (or, expressed another way, D293G-L294W, D293D-L294W, or D293G-L294L).

In the case of CET, the residues at positions P1′ and P2′ are also D-L, which are residues 293 and 294, respectively, of the native CET sequence, SEQ ID NO:1. Accordingly, in some embodiments, the residues at positions P1′ and P2′ of CET and CET-based immunotoxins of the invention are selected from the group consisting of G-W, D-W, and G-L (or, expressed another way, D293G-L294W, D293D-L294W, or D293G-L294L).

Any particular furin cleavable sequence can be readily tested by making it into an immunotoxin with, for example, the anti-transferrin receptor antibody used in the studies herein and testing the resulting immunotoxin in vitro on a transferrin receptor-positive cell line. In preferred embodiments, the furin cleavable sequences do not reduce the cytotoxicity of the resulting immunotoxin below 10% of the cytotoxicity of that of the same antibody-toxin chimeric protein when made with CT40 or CET40 and tested on the same cell line, and more preferably do not reduce the cytotoxicity of the resulting immunotoxin below 15%, 20%, 25%, 30% 40%, 50%, 60%, 70%, 75%, 80%, 90% or higher, with each increasing percentage of cytotoxicity being more preferred than the one preceding it.

Whether or not any particular sequence is cleavable by furin can be determined by methods known in the art. For example, whether or not a sequence is cleavable by furin can be tested by incubating the sequence with furin in furin buffer (0.2 M NaOAc (pH 5.5), 5 mM CaCl₂) at a 1:10 enzyme:substrate molar ratio at 25° C. for 16 hours. These conditions have previously been established as optimal for furin cleavage of PE and should also be suitable for testing furin cleavage for CT and CET.

Preferably, the furin used is human furin. Recombinant truncated human furin is commercially available, for example, from New England Biolabs (Beverly, Mass.). See also, Bravo et al., J Biol Chem, 269(14):25830-25837 (1994).

B. Production of Targeted Toxins

Targeted toxins of the invention include, but are not limited to, molecules in which there is a covalent linkage of a toxin molecule to an antibody or other targeting agent. The choice of a particular targeting agent depends on the particular cell to be targeted. With the toxin molecules provided herein, one of skill can readily construct a variety of clones containing functionally equivalent nucleic acids, such as nucleic acids which differ in sequence but which encode the same toxin and antibody sequence. Thus, the present invention provides nucleic acids encoding antibodies and toxin conjugates and fusion proteins thereof.

1. Recombinant Methods

The nucleic acid sequences of the present invention can be prepared as described herein or by any suitable method including, for example, cloning of appropriate sequences or by direct chemical synthesis by methods such as the phosphotriester method of Narang et al., Meth. Enzymol., 68:90-99 (1979); the phosphodiester method of Brown et al., Meth. Enzymol., 68:109-151 (1979); the diethylphosphoramidite method of Beaucage et al., Tetra. Lett., 22:1859-1862 (1981); the solid phase phosphoramidite triester method described by Beaucage & Caruthers, Tetra. Letts., 22(20):1859-1862 (1981), e.g., using an automated synthesizer as described in, for example, Needham-VanDevanter et al., Nucl. Acids Res., 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences. As described herein, a nucleic acid for CET was made synthetically (SEQ ID NOS:3 and 33) to allow optimal codon usage in E. coli.

In a preferred embodiment, the nucleic acid sequences of this invention are prepared by cloning techniques. Examples of appropriate cloning and sequencing techniques, and instructions sufficient to direct persons of skill through many cloning exercises are found in Sambrook et al., MOLECULAR CLONING: A LABORATORY MANUAL (2ND ED.), Vols. 1-3, Cold Spring Harbor Laboratory (1989)), Berger and Kimmel (eds.), GUIDE TO MOLECULAR CLONING TECHNIQUES, Academic Press, Inc., San Diego Calif. (1987)), or Ausubel et al. (eds.), CURRENT PROTOCOLS IN MOLECULAR BIOLOGY, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Nucleic acids encoding native PE, cholix toxin or cholera exotoxin can also be modified to form the targeted toxins of the present invention. Modification by site-directed mutagenesis is well known in the art. Nucleic acids encoding PE, cholix toxin or cholera exotoxin can be amplified by in vitro methods. Amplification methods include the polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In a preferred embodiment, targeted toxins are prepared by inserting the cDNA which encodes an antibody or other targeting moiety of choice, such as a cytokine, into a vector which comprises the cDNA encoding a desired cholix toxin. The insertion is made so that the targeting agent (for ease of discussion, the discussion herein will assume the targeting agent is an Fv, although other targeting agents could be substituted with equal effect) and the PE, cholix toxin or cholera exotoxin are read in frame, that is in one continuous polypeptide which contains a functional Fv region and a functional PE, cholix toxin or cholera exotoxin region. In a particularly preferred embodiment, cDNA encoding a PE, cholix toxin or cholera exotoxin is ligated to a scFv so that the toxin is located at the carboxyl terminus of the scFv. In other preferred embodiments, cDNA encoding a PE, cholix toxin or cholera exotoxin is ligated to a scFv so that the toxin is located at the amino terminus of the scFv.

Once the nucleic acids encoding a PE, cholix toxin or cholera exotoxin, antibody, or a targeted toxin are isolated and cloned, one may express the desired protein in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eucaryotic cells such as the COS, CHO, HeLa and myeloma cell lines. No attempt to describe in detail the various methods known for the expression of proteins in prokaryotes or eukaryotes will be made. In brief, the expression of natural or synthetic nucleic acids encoding the isolated proteins of the invention will typically be achieved by operably linking the DNA or cDNA to a promoter (which is either constitutive or inducible), followed by incorporation into an expression cassette. The cassettes can be suitable for replication and integration in either prokaryotes or eukaryotes. Typical expression cassettes contain transcription and translation terminators, initiation sequences, and promoters useful for regulation of the expression of the DNA encoding the protein. To obtain high level expression of a cloned gene, it is desirable to construct expression cassettes which contain, at the minimum, a strong promoter to direct transcription, a ribosome binding site for translational initiation, and a transcription/translation terminator. For E. coli this includes a promoter such as the T7, trp, lac, or lambda promoters, a ribosome binding site and preferably a transcription termination signal. For eukaryotic cells, the control sequences can include a promoter and preferably an enhancer derived from immunoglobulin genes, SV40, cytomegalovirus, and a polyadenylation sequence, and may include splice donor and acceptor sequences. The cassettes of the invention can be transferred into the chosen host cell by well-known methods such as calcium chloride transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

One of skill would recognize that modifications can be made to a nucleic acid encoding a polypeptide (i.e., PE, cholix toxin, cholera exotoxin or a targeted toxins formed from a PE, cholix toxin or cholera exotoxin) without diminishing its biological activity. Some modifications may be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly H is) to aid in purification steps.

In addition to recombinant methods, the targeted toxins and cholix toxin can also be constructed in whole or in part using standard peptide synthesis. Solid phase synthesis of the polypeptides of the present invention of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, THE PEPTIDES: ANALYSIS, SYNTHESIS, BIOLOGY. VOL. 2: SPECIAL METHODS IN PEPTIDE SYNTHESIS, PART A, pp. 3-284; Merrifield et al., J. Am. Chem. Soc., 85:2149-2156 (1963), and Stewart et al., SOLID PHASE PEPTIDE SYNTHESIS, 2ND ED., Pierce Chem. Co., Rockford, Ill. (1984). Proteins of greater length may be synthesized by condensation of the amino and carboxyl teunini of shorter fragments. Methods of forming peptide bonds by activation of a carboxyl terminal end (e.g., by the use of the coupling reagent N,N′-dicycylohexylcarbodiimide) are known to those of skill.

2. Purification

Once expressed, the recombinant targeted toxins can be purified as described herein or according to standard procedures of the art, including ammonium sulfate precipitation, affinity columns, column chromatography, and the like (see, generally, R. Scopes, PROTEIN PURIFICATION, Springer-Verlag, N.Y. (1982)). Substantially pure compositions of at least about 90 to 95% homogeneity are preferred, and 98 to 99% or more homogeneity are most preferred for pharmaceutical uses. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies of this invention. See, Buchner et al., Anal. Biochem., 205:263-270 (1992); Pluckthun, Biotechnology, 9:545 (1991); Huse et al., Science, 246:1275 (1989) and Ward et al., Nature, 341:544 (1989), all incorporated by reference herein.

Often, functional heterologous proteins from E. coli or other bacteria are isolated from inclusion bodies and require solubilization using strong denaturants, and subsequent refolding. During the solubilization step, as is well-known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena et al., Biochemistry, 9: 5015-5021 (1970), incorporated by reference herein, and especially as described by Buchner et al., supra.

Renaturation is typically accomplished by dilution (e.g., 100-fold) of the denatured and reduced protein into refolding buffer. An exemplary buffer is 0.1 M Tris, pH 8.0, 0.5 M L-arginine, 8 mM oxidized glutathione, and 2 mM EDTA.

As a modification to the two chain antibody purification protocol, the heavy and light chain regions are separately solubilized and reduced and then combined in the refolding solution. A preferred yield is obtained when these two proteins are mixed in a molar ratio such that a 5-fold molar excess of one protein over the other is not exceeded. It is desirable to add excess oxidized glutathione or other oxidizing low molecular weight compounds to the refolding solution after the redox-shuffling is completed.

### III. Pharmaceutical Compositions and Administration

In one aspect the present invention provides a pharmaceutical composition or a medicament comprising at least one chimeric protein of the present invention, preferably a targeted toxin, and optionally a pharmaceutically acceptable carrier. A pharmaceutical composition or medicament can be administered to a patient for the treatment of a condition, including, but not limited to, a malignant disease or cancer.

A. Formulation

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. The chimeric proteins of the present invention can be formulated for administration by any suitable route, including via inhalation, topically, nasally, orally, parenterally, or rectally. Thus, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets and capsules can be administered orally, rectally or vaginally.

The compositions for administration will commonly comprise a solution of the chimeric protein, preferably a targeted toxin, dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

The targeted toxin compositions of this invention (i.e., PE, CT or CET linked to an antibody or other targeting agent) are particularly useful for parenteral administration, such as intravenous administration or administration into a body cavity.

The chimeric proteins, preferably targeted toxins, of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

Controlled release parenteral formulations of the targeted toxin compositions of the present invention can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter J., COLLOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp. 315-339 (1992), both of which are incorporated herein by reference.

Polymers can be used for ion-controlled release of targeted toxin compositions of the present invention. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res., 9:425-434 (1992); and Pec et al., J. Parent. Sci. Tech., 44(2):58-65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

Suitable formulations for transdermal application include an effective amount of a composition of the present invention with a carrier. Preferred carriers include absorbable pharmacologically acceptable solvents to assist passage through the skin of the host. For example, transdermal devices are in the form of a bandage comprising a backing member, a reservoir containing the composition optionally with carriers, optionally a rate controlling barrier to deliver the composition to the skin of the host at a controlled and predetermined rate over a prolonged period of time, and means to secure the device to the skin. Matrix transdermal formulations may also be used.

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., a composition of the present invention, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active composition.

For administration by inhalation the chimeric protein, preferably an antibody and/or targeted toxin may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, 1,1,1,2-tetrafluorethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the chimeric protein, preferably an antibody and/or targeted toxin and a suitable powder base, for example, lactose or starch.

The compositions can also be formulated in rectal compositions, for example, suppositories or retention enemas, for example, containing conventional suppository bases, for example, cocoa butter or other glycerides.

Furthermore, the compositions can be formulated as a depot preparation. Such long-acting formulations can be administered by implantation (for example, subcutaneously or intramuscularly) or by intramuscular injection. Thus, for example, the composition can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

B. Dosage

In one embodiment of the present invention, a pharmaceutical composition or medicament is administered to a patient at a therapeutically effective dose to prevent, treat, or control a disease or malignant condition, such as cancer. The pharmaceutical composition or medicament is administered to a patient in an amount sufficient to elicit an effective therapeutic or diagnostic response in the patient. An effective therapeutic or diagnostic response is a response that at least partially arrests or slows the symptoms or complications of the disease or malignant condition. An amount adequate to accomplish this is defined as “therapeutically effective dose.”

The dosage of chimeric proteins, preferably targeted toxins, or compositions administered is dependent on the species of warm-blooded animal (mammal), the body weight, age, individual condition, surface area of the area to be treated and on the form of administration. The size of the dose also will be determined by the existence, nature, and extent of any adverse effects that accompany the administration of a particular compound in a particular subject. A unit dosage for administration to a mammal of about 50 to 70 kg may contain between about 5 and 500 mg of the active ingredient. Typically, a dosage of the compound of the present invention, is a dosage that is sufficient to achieve the desired effect.

Optimal dosing schedules can be calculated from measurements of chimeric protein, preferably targeted toxin, accumulation in the body of a subject. In general, dosage is from 1 ng to 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of a chimeric protein, preferably a targeted toxin, to a human being following established protocols known in the art and the disclosure herein.

Optimum dosages, toxicity, and therapeutic efficacy of compositions may vary depending on the relative potency of individual compositions and can be determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio, LD₅₀/ED₅₀. Compositions that exhibit large therapeutic indices are preferred. While compositions that exhibit toxic side effects can be used, care should be taken to design a delivery system that targets such compositions to the site of affected tissue to minimize potential damage to normal cells and, thereby, reduce side effects.

The data obtained from, for example, animal studies (e.g. rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a chimeric protein, preferably a targeted toxin is from about 1 ng/kg to 100 mg/kg for a typical subject.

A typical targeted toxin composition of the present invention for intravenous administration would be about 0.1 to 10 mg per patient per day. Dosages from 0.1 up to about 100 mg per patient per day may be used. Actual methods for preparing administrable compositions will be known or apparent to those skilled in the art and are described in more detail in such publications as REMINGTON'S PHARMACEUTICAL SCIENCE, 19TH ED., Mack Publishing Company, Easton, Pa. (1995).

Exemplary doses of the compositions described herein, include milligram or microgram amounts of the composition per kilogram of subject or sample weight (e.g., about 1 microgram per-kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In one embodiment of the present invention, a pharmaceutical composition or medicament comprising a chimeric protein, preferably a targeted toxin, of the present invention is administered, e.g., in a daily dose in the range from about 1 mg of compound per kg of subject weight (1 mg/kg) to about 1 g/kg. In another embodiment, the dose is a dose in the range of about 5 mg/kg to about 500 mg/kg. In yet another embodiment, the dose is about 10 mg/kg to about 250 mg/kg. In another embodiment, the dose is about 25 mg/kg to about 150 mg/kg. A preferred dose is about 10 mg/kg. The daily dose can be administered once per day or divided into subdoses and administered in multiple doses, e.g., twice, three times, or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

Following successful treatment, it may be desirable to have the subject undergo maintenance therapy to prevent the recurrence of the disease or malignant condition treated.

C. Administration

The compositions of the present invention can be administered for therapeutic treatments. In therapeutic applications, compositions are administered to a patient suffering from a disease or malignant condition, such as cancer, in an amount sufficient to cure or at least partially arrest the disease and its complications. An amount adequate to accomplish this is defined as a “therapeutically effective dose.” Amounts effective for this use will depend upon the severity of the disease and the general state of the patient's health. An effective amount of the compound is that which provides either subjective relief of a symptom(s) or an objectively identifiable improvement as noted by the clinician or other qualified observer.

Single or multiple administrations of the compositions are administered depending on the dosage and frequency as required and tolerated by the patient. In any event, the composition should provide a sufficient quantity of the proteins of this invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

To achieve the desired therapeutic effect, compositions may be administered for multiple days at the therapeutically effective daily dose. Thus, therapeutically effective administration of compositions to treat a disease or malignant condition described herein in a subject may require periodic (e.g., daily) administration that continues for a period ranging from three days to two weeks or longer. Typically, compositions will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the compounds or compositions are not administered daily, so long as the administration is repeated frequently enough to maintain a therapeutically effective concentration of the composition in the subject. For example, one can administer a composition every other day, every third day, or, if higher dose ranges are employed and tolerated by the subject, once a week.

In some embodiments, of the method of inhibiting the growth of a population of cells bearing one or more cell surface marker, a second chimeric protein is administered to said population of cells about three weeks after administration of the first chimeric protein to the population of cells. In some embodiments, the second chimeric protein is administered to the population of cells within about one month of administration of the first chimeric protein to the population of cells. In some embodiments, the second chimeric protein is administered to the population of cells within about two months of administration of the first chimeric protein to the population of cells.

Among various uses of the targeted toxins of the present invention are included a variety of disease conditions caused by specific human cells that may be eliminated by the toxic action of the fusion protein. For example, the targeted cells might express a cell surface marker such as mesothelin or CD22.

### IV. Methods of Using Compositions

The compositions of the present invention find use in a variety of ways. For example, the present invention provides methods for using the compositions of the present invention to (i) induce apoptosis in a cell bearing one or more surface markers (ii) inhibit unwanted growth, hyperproliferation or survival of a cell bearing one or more cell surface markers, (iii) treat a condition, such as a cancer, and (iv) provide therapy for a mammal having developed antibodies to Pseudomonas exotoxin A, and (v) provide therapy for a mammal having developed a disease caused by the presence of cells bearing one or more cell surface marker.

Any cell or tumor cell expressing one or more cell surface marker, preferably a cell surface receptor, can be used to practice a method of the present invention. A preferred cell or tumor cell expressing a surface marker is s selected from the group consisting of neuroblastoma, intestine carcinoma, rectum carcinoma, colon carcinoma, familiary adenomatous polyposis carcinoma, hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors, glioblastoma, astrocytoma, meningioma, medulloblastoma, peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroids melanoma, seminoma, rhabdomyosarcoma, craniopharyngeoma, osteosarcoma, chondrosarcoma, myosarcome, liposarcoma, fibrosarcoma, Ewing sarcoma, and plasmocytoma.

Methods of the present invention can be practiced in vitro or in vivo. When referring to a cell, it is understood that that this term also includes a population of cells, i.e., more than one cell.

A. Using Compositions for Inducing Apoptosis in a Cell Bearing One or More Cell Surface Marker

Apoptosis plays a central role in both the development and homeostasis of multicellular organisms. “Apoptosis” refers to programmed cell death and is characterized by certain cellular characteristics, such as membrane blobbing, chromatin condensation and fragmentation, formation of apoptotic bodies and a [positive “TUNEL” (terminal deoxynucleotidyl transferase-mediated UTP nick end-labeling) staining pattern. A later step in apoptotic process is the degradation of the plasma membrane, rendering apoptotic cells leaky to various dyes (e.g., propidium iodide).

Apoptosis can be induced by multiple independent signaling pathways that converge upon a final effector mechanism consisting of multiple interactions between several death receptors and their ligands, which belong to the tumor necrosis factor (TNF) receptor/ligand superfamily. The best-characterized death receptors are CD95 (“Fas”), TNFR1 (p55), death receptor 3 (DR3 or Apo3/TRAMO), DR4 and DR5 (apo2-TRAIL-R2). The final effector mechanism of apoptosis is the activation of a series of proteinases designated as caspases. The activation of these caspases results in the cleavage of a series of vital cellular proteins and cell death.

The present invention provides methods for inducing apoptosis in a cell expressing one or more cell surface marker. In one aspect, the method for inducing apoptosis in a cell comprises the step of exposing the cell expressing one or more cell surface marker, such as a cell surface receptor, to a composition or contacting the cell with a composition comprising a chimeric protein, preferably a targeted toxin, of the present invention. In a preferred embodiment, the composition comprises a PE- CT-, and/or CET-based targeted toxin, preferably a PE- CT-, and/or CET-based immunotoxin. Typically, the cells are exposed to or contacted with an effective amount of the composition wherein the contacting results in inducing apoptosis.

In another aspect of present invention, a method of inducing a tumor cell expressing one or more cell surface marker to undergo apoptosis is provided comprising the step of administering a chimeric protein, preferably a targeted toxin, of the present invention. In a preferred embodiment, the chimeric protein is a PE- CT-, and/or CET-based immunotoxin.

B. Using Compositions for Inhibiting Growth, Hyperproliferation, or Survival of a Cell Bearing One or More Cell Surface Marker

It is an object of the present invention to provide novel therapeutic strategies for treatment of cancers using the compositions of the invention. In one aspect of the present invention, a method for inhibiting at least one of unwanted growth, hyperproliferation, or survival of a cell is provided. In one embodiment, this method comprises the step of determining whether the cell expresses one or more cell surface marker, preferably a cell surface receptor. This method also comprises the step of contacting the cell with an effective amount of a composition of the present invention, wherein the step of contacting results in the inhibition of at least one of unwanted growth, hyperproliferation, or survival of the cell. Preferred cancer cells are described herein.

In a preferred embodiment, the composition comprises a PE- CT-, and/or CET-based targeted toxin, preferably a PE- CT-, and/or CET-based immunotoxin. Typically, the cells are exposed to or contacted with an effective amount of the composition wherein the contacting results in the inhibition of at least one of unwanted growth, hyperproliferation, or survival of the cell.

Thus, in one aspect of the present invention methods of inhibiting growth of a population of cells bearing one or more cell surface markers are provided. In a preferred embodiment, this method comprises the steps of (a) contacting a population of cells with a first chimeric protein comprising (i) a targeting moiety which specifically binds at least one of the surface markers and (ii) a toxin selected from Pseudomonas exotoxin A (PE), cholix toxin (CT) and cholera exotoxin (CET), and (b) contacting the population of cells with a second chimeric protein comprising (i) a targeting moiety which specifically binds at least one of the surface markers and (ii) a toxin selected from a PE, a CT and a CET, wherein the toxin of the second chimeric protein is not the same toxin comprising part of the first chimeric protein. Thereby the growth of the population of cells is inhibited.

In some embodiments, the toxin of the first chimeric protein is PE and the toxin of the second chimeric protein is CT or CET. In some embodiments, the toxin of the first chimeric protein is CT or CET and the toxin of the second chimeric protein is PE.

In some embodiments, the second chimeric protein is administered to said population of cells about three weeks after administration of the first chimeric protein to the population of cells. In some embodiments, the second chimeric protein is administered to the population of cells within about one month of administration of the first chimeric protein to the population of cells. In some embodiments, the second chimeric protein is administered to the population of cells within about two months of administration of the first chimeric protein to the population of cells.

Many tumors form metastasis. Thus, in another aspect of the present invention, the compositions of the present invention are used to prevent the formation of a metastasis. This method comprises the step of administering to a tumor cell a composition of the present invention wherein the administering results in the prevention of metastasis. In a preferred embodiment, the composition comprises a PE- CT-, and/or CET-based targeted toxin, preferably a PE- CT-, and/or CET-based immunotoxin. Typically, the cells are exposed to or contacted with an effective amount of the composition wherein the contacting results in the prevention of metastasis.

C. Using Compositions for Treating Cancer

Methods of the present invention can be practiced in vitro and in vivo. Thus, in another aspect of the present invention, a method for treating a subject suffering from a cancerous condition is provided. This method comprises the step of administering to a subject having been diagnosed with a cancer a therapeutically effective amount of a composition of the present invention, wherein the cancerous condition is characterized by unwanted growth or proliferation of a cell expressing one or more cell surface marker, and wherein the step of administering results in the treatment of the subject.

In a preferred embodiment, the composition comprises a PE- CT-, and/or CET-based targeted toxin, preferably a PE- CT-, and/or CET-based immunotoxin. Typically, the cells are exposed to or contacted with an effective amount of the composition wherein the contacting results in the treatment of the subject.

Compositions of the present invention can be used to treat any cancer described herein. In one embodiment of the present invention, a composition of the present invention is used to treat a subject suffering from a lung cancer expressing one or more cell surface marker. A lung cancer includes, but is not limited to, bronchogenic carcinoma [squamous cell, undifferentiated small cell, undifferentiated large cell, adenocarcinoma], alveolar [bronchiolar] carcinoma, bronchial adenoma, sarcoma, lymphoma, chondromatous hamartoma, mesothelioma, SCLC, and NSCLC.

In another embodiment of the present invention, a composition of the present invention is used to treat a subject suffering from a sarcoma expressing one or more cell surface marker. A sarcoma includes, but is not limited to, cancers such as angiosarcoma, fibrosarcoma, rhabdomyosarcoma, liposarcoma, myxoma, rhabdomyoma, fibroma, lipoma and teratoma.

In yet another embodiment of the present invention, a composition of the present invention is used to treat a subject suffering from a gastrointestinal cancer expressing one or more cell surface marker. A gastrointestinal cancer includes, but is not limited to cancers of esophagus [squamous cell carcinoma, adenocarcinoma, leiomyosarcoma, lymphoma], stomach [carcinoma, lymphoma, leiomyosarcoma], pancreas [ductal adenocarcinoma, insulinoma, glucagonoma, gastrinoma, carcinoid tumors, VIPoma], small bowel [adenocarcinoma, lymphoma, carcinoid tumors, Kaposi's sarcoma, leiomyoma, hemangioma, lipoma, neurofibroma, fibroma], and large bowel [adenocarcinoma, tubular adenoma, villous adenoma, hamartoma, leiomyoma].

In one embodiment of the present invention, a composition of the present invention is used to treat a subject suffering from a cancer of the genitourinary tract expressing one or more cell surface marker. Cancers of the genitourinary tract include, but are not limited to cancers of kidney [adenocarcinoma, Wilms tumor (nephroblastoma), lymphoma, leukemia, renal cell carcinoma], bladder and urethra [squamous cell carcinoma, transitional cell carcinoma, adenocarcinoma], prostate [adenocarcinoma, sarcoma], and testis [seminoma, teratoma, embryonal carcinoma, teratocarcinoma, choriocarcinoma, sarcoma, Leydig cell tumor, fibroma, fibroadenoma, adenomatoid tumors, lipoma].

In another embodiment of the present invention, a composition of the present invention is used to treat a subject suffering from a liver cancer expressing one or more cell surface marker. A liver cancer includes, but is not limited to, hepatocellular carcinoma, cholangiocarcinoma, hepatoblastoma, angiosarcoma, hepatocellular adenoma, and hemangioma.

In one embodiment of the present invention, a composition of the present invention is used to treat a subject suffering from a skin cancer expressing one or more cell surface marker. Skin cancer includes, but is not limited to, malignant melanoma, basal cell carcinoma, squamous cell carcinoma, Kaposi's sarcoma, nevi, dysplastic nevi, lipoma, angioma, dennatofibroma, keloids, and psoriasis.

In one embodiment of the present invention, a composition of the present invention is used to treat a subject suffering from a gynecological cancer expressing one or more cell surface marker. Gynecological cancers include, but are not limited to, cancer of uterus [endometrial carcinoma], cervix [cervical carcinoma, pre-invasive cervical dysplasia], ovaries [ovarian carcinoma (serous cystadenocarcinoma, mucinous cystadenocarcinoma, endometrioid carcinoma, clear cell adenocarcinoma, unclassified carcinoma), granulosa-theca cell tumors, Sertoli-Leydig cell tumors, dysgeHninoma, malignant teratoma and other gemi cell tumors], vulva [squamous cell carcinoma, intraepithelial carcinoma, adenocarcinoma, fibrosarcoma, melanoma], vagina [clear cell carcinoma, squamous cell carcinoma, sarcoma botryoides (embryonal rhabdomyosarcoma), and fallopian tubes [carcinoma].

In yet another embodiment of the present invention, a composition of the present invention is used to treat a subject suffering from a bone cancer expressing one or more cell surface marker. Bone cancer includes, but is not limited to, osteogenic sarcoma [osteosarcoma], fibrosarcoma, malignant fibrous histiocytoma, chondrosarcoma, Ewing's sarcoma, malignant lymphoma [reticulum cell sarcoma], multiple myeloma, malignant giant cell tumor, chordoma, osteochondroma [osteocartilaginous exostoses], benign chondroma, chondroblastoma, chondromyxoid fibroma, osteoid osteoma, and giant cell tumors.

In one embodiment of the present invention, a composition of the present invention is used to treat a subject suffering from a cancer of the nervous system expressing one or more cell surface marker. Cancers of the nervous system include, but are not limited to cancers of skull [osteoma, hemangioma, granuloma, xanthoma, Paget's disease of bone], meninges [meningioma, meningiosarcoma, gliomatosis], brain [astrocytoma, medulloblastoma, glioma, ependymoma, germinoma (pinealoma), glioblastoma multiforme, oligodendroglioma, schwannoma, retinoblastoma, congenital tumors], and spinal cord [neurofibroma, meningioma, glioma, sarcoma].

In one embodiment of the present invention, a composition of the present invention is used to treat a subject suffering from a hematologic cancer expressing one or more cell surface marker. Hematologic cancers include, but are not limited to cancer of blood [myeloid leukemia (acute and chronic), acute lymphoblastic leukemia, chronic lymphocytic leukemia, myeloproliferative diseases, multiple myeloma, myelodysplastic syndrome], Hodgkin's disease, and non-Hodgkin's lymphoma (malignant lymphoma).

In one embodiment of the present invention, a composition of the present invention is used to treat a subject suffering from a cancer of adrenal glands expressing one or more cell surface marker. A cancer of adrenal glands includes, but is not limited to, neuroblastoma.

Methods for treating cancer may optionally comprise one or more of the following steps: obtaining a biological sample of tissue or fluid from an individual; screening the biological sample for the expression of one or more cell surface marker, preferably a cell surface receptor, for example by contacting the biological sample with an antibody directed to the surface marker, preferably a cell surface receptor; or screening the biological sample for expression of a surface marker polynucleotide, preferably a cell surface receptor polynucleotide, for example by detecting a surface marker mRNA, preferably, a cell surface receptor mRNA. This can be done using standard technologies known in the art, e.g., Western blotting, Northern blotting or PCR.

D. Using Compositions for Treating a Subject Having Developed Neutralizing Antibodies to Pseudomonas Exotoxin A

Many cancers are initially treated using a toxin develop resistance against such toxin which then is not longer effective. Resistance against a toxin can be due by a patient developing neutralizing antibodies against such toxin. Thus, another aspect of the present invention, a method for treating a subject having developed neutralizing antibodies to Pseudomonas Exotoxin A is provided.

In a preferred embodiment, this method comprises the steps of (a) selecting a mammal having developed neutralizing antibodies to Pseudomonas exotoxin A; (b) administering to said mammal a chimeric protein comprising (i) a targeting moiety which specifically binds to at least one surface marker on a cell within said mammal; and (ii) cholix toxin (CT) or cholera exotoxin (CET).

In a preferred embodiment, the chimeric protein comprises a PE- CT-, and/or CET-based targeted toxin, preferably a PE- CT-, and/or CET-based immunotoxin. Typically, the cells are exposed to or contacted with an effective amount of the composition wherein the contacting results in the treatment of the subject.

The invention further provides the use of targeted toxins employing cholix toxins and exotoxin as the toxic portion before or after the use of targeted toxins employing Pseudomonas exotoxin A as the toxic portion.

E. Using Compositions for Treating a Subject Having Developed a Disease Caused by the Presence of Cells Bearing One or More Cell Surface Markers

Also provided is a method a method of providing therapy for a mammal having developed a disease caused by the presence of cells bearing one or more cell surface markers. In a preferred embodiment, this method comprises the steps of (a) administering to said mammal a chimeric protein comprising (i) a targeting moiety which specifically binds to at least one surface marker on said cells and (ii) a cholix toxin (CT) or a cholera exotoxin (CET) and (b) administering to said mammal a chimeric protein comprising (i) a targeting moiety which specifically binds to at least one surface marker on said cells and (ii) Pseudomonas exotoxin A toxin. Step (a) of this method can be performed before or after step (b).

In a preferred embodiment, the chimeric protein comprises a PE- CT-, and/or CET-based targeted toxin, preferably a PE- CT-, and/or CET-based immunotoxin. Typically, the cells are exposed to or contacted with an effective amount of the composition wherein the contacting results in the treatment of the subject.

In another embodiment, this invention provides for eliminating target cells in vitro or ex vivo using PE, CT and CET toxins of the present invention. For example, immunotoxins comprising a PE, CT, or CET toxin can be used to purge targeted cells from a population of cells in a culture. Thus, for example, cells cultured from a patient having a cancer expressing CD22 can be purged of cancer cells by contacting the culture with immunotoxins which use anti-CD22 antibodies as a targeting moiety.

In some instances, the target cells may be contained within a biological sample. A “biological sample” as used herein is a sample of biological tissue or fluid that contains target cells and non-target cells. Such samples include, but are not limited to, tissue from biopsy, blood, and blood cells (e.g., white cells). A biological sample is typically obtained from a multicellular eukaryote, preferably a mammal such as rat, mouse, cow, dog, guinea pig, or rabbit, and more preferably a primate, such as a macaque, chimpanzee, or human. Most preferably, the sample is from a human.

### V. Kits, Containers, Devices, and Systems

For use in diagnostic, research, and therapeutic applications described above, kits and systems are also provided by the invention. In the diagnostic and research applications such kits and systems may include any or all of the following: assay reagents, buffers, a composition of the present invention, a PE polypeptide, a CT polypeptide, a CET polypeptide, a PE nucleic acid, a CT nucleic acid, a CET nucleic acid, a PE expression vector, a CT expression vector, a CET expression vector, a genetically modified eukaryotic cell comprising a nucleic acid, polypeptide or expression vector for PE, CT or CET as described, etc. A therapeutic product may include sterile saline or another pharmaceutically acceptable emulsion and suspension base.

In addition, the kits and systems may include instructional materials containing directions (i.e., protocols) for the practice of the methods of this invention. The instructions may be present in the subject kits in a variety of forms, one or more of which may be present in the kit. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

A wide variety of kits, systems, and compositions can be prepared according to the present invention, depending upon the intended user of the kit and system and the particular needs of the user.

In a preferred embodiment of the present invention, the kit or system comprises a composition of the present invention, preferably a nucleic acid encoding a PE-, CT-, or CET-polypeptide, more preferably a nucleic acid encoding a PE-, CT-, or CET-based immunotoxin.

In another preferred embodiment of the present invention, the kit or system comprises a composition of the present invention, preferably an isolated PE-, CT-, or CET-polypeptide, more preferably a PE-, CT-, or CET-based immunotoxin. Preferably, the isolated PE-, CT-, or CET-polypeptide or the isolated PE-, CT-, or CET-based immunotoxin is a recombinant polypeptide.

In yet another preferred embodiment of the present invention, the kit or system comprises a composition of the present invention, preferably an expression vector encoding a PE-, CT-, or CET-polypeptide.

The kits or systems according to the present invention may further comprise a reagent for assessing the effectiveness or activity of a chimeric protein, preferably a targeted toxin of the present invention. Such reagents are described herein and are well known to those skilled in the art.

Kits with unit doses of the active composition, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the composition in treating a disease or malignant condition. Suitable active compositions and unit doses are those described herein above.

Although the forgoing invention has been described in some detail by way of illustration and example for clarity and understanding, it will be readily apparent to one of ordinary skill in the art in light of the teachings of this invention that certain variations, changes, modifications and substitutions of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results. It is also to be understood that the terminology used herein is for the purpose of describing particular embodiments only, and is not intended to be limiting, since the scope of the present invention will be limited only by the appended claims.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present invention has a wide variety of applications. The invention is further illustrated by the following examples, which are only illustrative and are not intended to limit the definition and scope of the invention in any way.

### VI. Examples

### Example 1

### General

Surprisingly, the studies underlying the invention show that the PE and CT toxins are functionally similar, but immunologically distinct. Despite the sequence and structural similarities noted above, none of the anti-PE polyclonal or monoclonal antibodies tested in the studies underlying the present invention have neutralized, or even significantly affected, the ability of CT-based immunotoxins to kill targeted cells.

A series of studies were conducted creating immunotoxins employing an exemplar antibody, an anti-transferrin receptor antibody known as HB21, and a population of human DLD-1 colon carcinoma cells, which express the transferrin receptor.

a) Preparation of Inclusion Bodies (IB)

Frozen cells (up to 10000 OD units) were resuspended in 25 ml of TE 50/20 (mM/mM, pH 8.0) and dispersed using a tissuemizer, a laboratory blender. Cells were then lysed with the addition of chicken egg white lysozyme (Sigma) to a final concentration of 200 μg/ml for 1 hr at RT. Lysed cells were incubated further for 30 min with the addition of 3.3 ml of 5.0 M NaCl and 3.3 ml of 25% Triton X-100. Inclusion bodies were then recovered in the pellet following centrifugation for 45 min. at 15,000×g (Sorvall SS-34 rotor). The pellet was resuspended in 25 ml TE 50/20, 1% vol/vol Triton X-100, dispersed using a tissuemizer and centrifuged as above three more times. To remove the detergent, the inclusion bodies were washed four times in TE 50/20. The IB pellet preparation was stored frozen at −80° C. until further protein purification.

b) Solubilization of Inclusion Bodies (IB)

Inclusion bodies were solubilized initially in 6 M Guanidine-HCl, 0.1 M Tris-HCl, 2 mM EDTA at pH 8.0. After 1-4 hr, dithioerythretol (DTE) was added to a final concentration of 65 mM (10 mg/ml) and solubilization allowed to proceed on a rocking platform overnight at RT.

c) Renaturation and Refolding

Solubilized immunotoxin was centrifuged to remove non-soluble material and the supernatant diluted (˜1:100 vol/vol) into a refolding buffer: 0.1 M Tris, 0.5 M L-Arginine-HCl, 2 mM EDTA, 0.9 mM GSSG, pH 8.0 at 10° C. After 24 hr, additional GSSG, (9 mM final), was added for another 24 hr. The refolded protein was then dialyzed against 20 mM Tris-HC1,100 mM Urea pH 8.0.

d) Anion Exchange Chromatography

The post dialysis immunotoxin preparation was adjusted to 2 L with deionized water and batch-adsorbed onto 50 ml of Q-sepharose. The resin was recovered on a 2 L Buchner funnel and washed with four volumes of 20 mM Tris-HCl, 1 mM EDTA, pH 8.0 and then eluted using the same buffer supplemented with 0.1M, 0.35M, and 0.5M NaCl. The immunotoxin eluting with 0.35M NaCl was retained for additional chromatography. The retained material was diluted in low salt buffer (buffer A, 20 mM Tris, pH 8.0) and pumped onto Mono-Q 5/5 column at 1 ml/min. Protein was eluted from the resin with a linear gradient 0-100% of buffer B (20 mM Tris pH 8.0, 0.0 M NaCl), over 30 ml, collecting 1 ml fractions. Peak fractions were concentrated using an Amicon Ultra (10,000 MWCO) concentrator (Amicon) to a volume <1 ml for gel filtration

e) Gel Filtration Chromatography

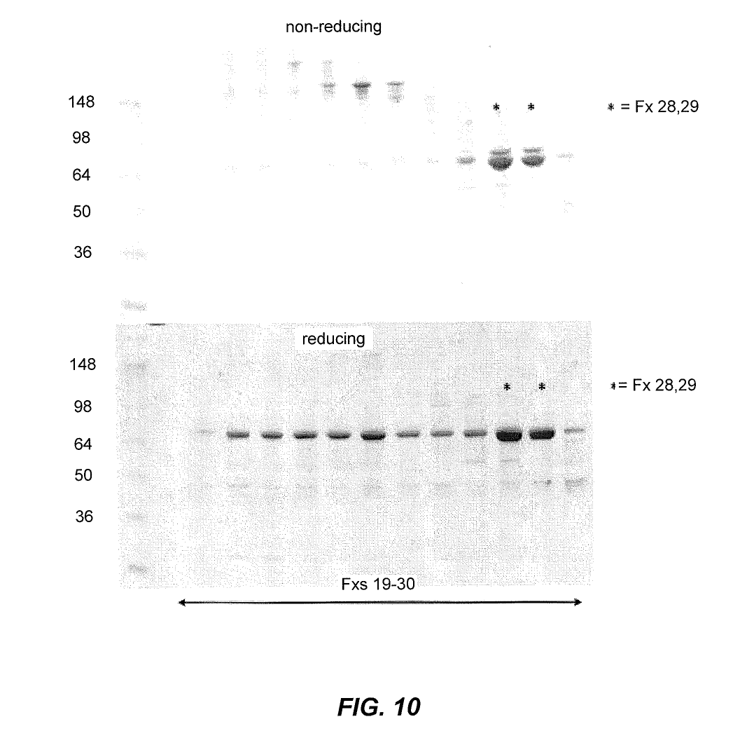

A concentrated immunotoxin protein sample obtained from the anion exchange chromatography (see above) was loaded onto a TSKgel G3000-SWx1 column (Tosoh Bioscience) at 0.5 ml/min, using PBS, pH 7.4, as the mobile phase. Fractions of 0.5 ml were collected and analyzed by SDS-PAGE (FIG. 10).

f) Cell Lines

Cells used herein were obtained from the American Type Culture Collection. KB3-1, A549, DLD-1, Raji, 293TT, HUT102 and L929 cells were grown in RPMI-1640 or DMEM and 10% fetal bovine serum supplemented with penicillin, streptomycin, glutamine and pyruvate.

g) Cytotoxicity Assay

The WST-1 (Roche) was used to assess cytotoxicity. Cells were seeded in 96-well plates at 5×10³ per well. After 24 hr, immunotoxins or immunotoxin-antibody mixtures were added to cells for a further 48 hr. Dye-containing media was removed and replaced with a 10% vol/vol of WST-1 reagent in dye-free RPMI-1640 growth media. Absorbance measurements were made at 450 nm at 30 min and 60 min. Replicates of 5 were used for each data point and all experiments were conducted independently at least twice. Cycloheximide was added at 10 μg/ml as a positive control in all experiments. For competition experiments with excess antibody, DLD-1 cells were pretreated with 10 μg/ml of HB21 for 30 min and then HB21scFv-CET40 was added to a final concentration of either 10 or 1 ng/ml.

h) Anti-PE Antibodies

Several anti-PE antibody preparations were evaluated. A mouse monoclonal antibody, termed M40-1, was originally described as a neutralizing antibody that also recognized PE via Western blots (Ogata et al., 1991, Infection and Immunity 59:407-14). Two rabbit polyclonal antibody preparations that reacted with PE via Western blot and neutralized the toxin were also employed. One of these was a lab reagent originally generated to formaldehyde treated native PE. The other was purchased from Sigma (P2318). Human sera from patients treated with the immunotoxin SS1P-PE38 (see herein) was also used.

i) Anti-CET40 Antibodies

To make antibodies reactive for CET40, a rabbit was hyperimmunized with an enzymatically inactive form of CET40 (E581A) fused to HB21scFv. CET40 (E581A) includes a mutation E to A at position581 in the NAD binding site to render the protein enzymatically inactive. As such, this CET(E581A)-based immunotoxin could be used to immunize a rabbit without killing it. Immunizations and antisera production were carried out at Convance Inc. Because these sera contained antibodies to both the scFv and CET40, Western blots were conducted on full length PE and CET proteins. Both PE and CET were expressed in E. coli and purified using the same protocol used to prepare HB21scFv-CET40.

j) Western Blots

Immunotoxin proteins, both HB21scFv-PE40 (“HB21-PE40”) or HB21-scFv-PCET40 (“HB21-CET40”) (30 ng) were separated via SDS-PAGE (8-16% gradient), transferred to PVDF membranes and probed with anti-PE antibodies. CET and PE toxins (without the scFV part) were similarly separated and transferred to PVDF membranes and analyzed with anti-CET polyclonal antibodies. Either donkey anti-mouse IgG-HRP or donkey anti-rabbit IgG-HRP (Jackson, Immunoresearch) were used to detect the primary antibodies. Reactive bands were detected by ECL and visualized on Amersham Hyperfilm.

k) Neutralization Assay

Rabbit (“Sigma” or “NCI”) or mouse (“M40-1”) antibodies were diluted 1:100 or to 20 μg/ml and mixed with either 5 or 1 ng/ml of either HB21-PE40 or HB21-CET40 for 1 hr at room temperature. At the end of the incubation the immunotoxin-antibody mixture was diluted 1:1 with media over cells. Cells were incubated with immunotoxin and antibodies for 48 hr and then evaluated for viability using the WST-1 assay.

Human sera (from 4 individuals) were obtained with informed consent before and after treatment with the PE38 immunotoxin, SS1P, which is directed to the surface antigen mesothelin (Hassan et al., 2007, Clin Cancer Res 13:5144-9). Immunotoxin treatment was at the dose level of 45 μg/kg for each the four individuals (Hassan et al., 2007, Clin Cancer Res 13:5144-9). The post treatment sample was documented as having neutralizing titers to PE38. The immunotoxin at 5 ng/ml or 1 ng/ml was mixed with a 1:100 dilution of patient sera and incubated for one hour at room temperature. After this incubation, 50 μL was added to each well giving a final immunotoxin concentration of 2.5 ng/ml and 0.5 ng/ml respectively and a final serum dilution of 1:200.

### Example 2

### Construction and Characterization of CET40 Immunotoxins

Evidence that certain strains of Vibrio cholerae encode an exotoxin similar to PE from Pseudomonas has been supported by: tissue culture experiments, bioinformatic comparisons of sequenced genomes and direct structural comparisons of the two toxins (Jorgensen et al., 2008, J Biol Chem 283:10671-8; Purdy et al., 2005, J Bacteriol 187:2992-3001; Dalsgaard et al., 1995, J Clin Microbiol 33:2715-22). Herein the functional similarity was confirmed by showing that domains II and III of CET can be used to generate immunotoxins with cell killing activities roughly equivalent to that of PE-based proteins. While a role for cholera exotoxin in contributing to human disease has not been firmly established, at least one report documents an outbreak of diarrhea caused by Vibrio cholerae isolate (strain 1587) that was negative for the structural genes encoding classical cholera toxin and positive for the CET (Dalsgaard et al., 1995, J Clin Microbiol 33:2715-22). With several Vibrio cholerae strains to choose from, Applicants focused on strain 1587 (Dalsgaard et al., 1995, J Clin Microbiol 33:2715-22) because it had been isolated from a disease outbreak rather than cholix toxin that had been derived from an environmental isolate. CET and cholix toxin differ by 14 amino acids. The consequence of this difference is not known, however, it may be speculated that one or more of these amino acid exchanges confers higher cytotoxic activity and/or less immunogenicity

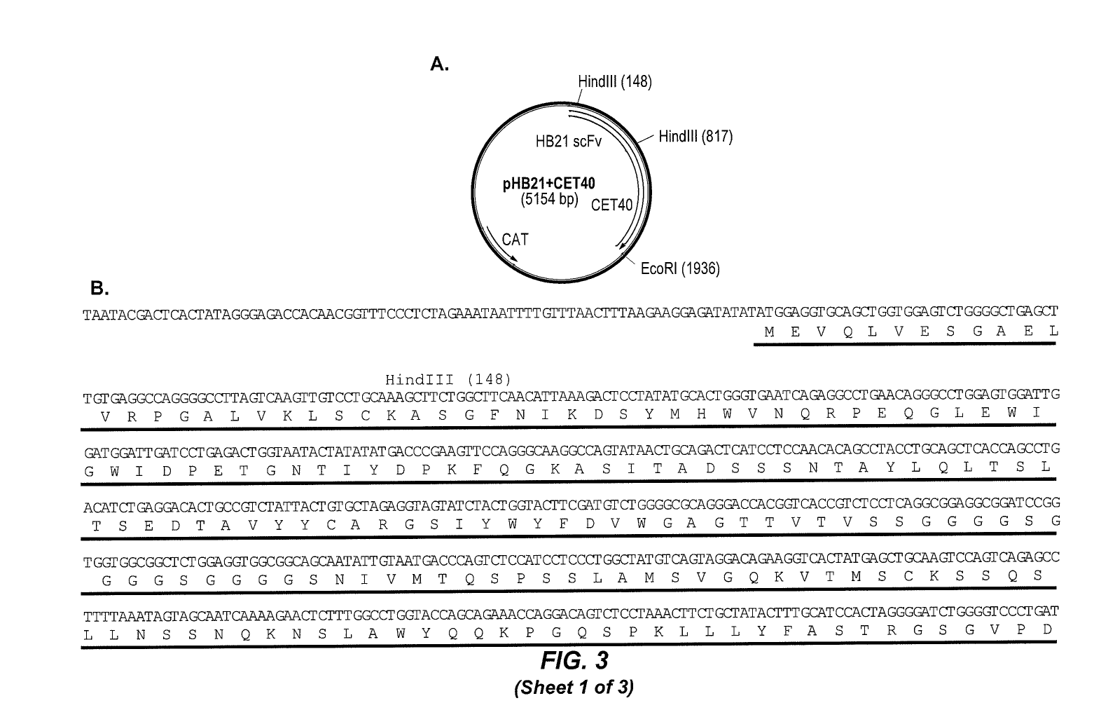

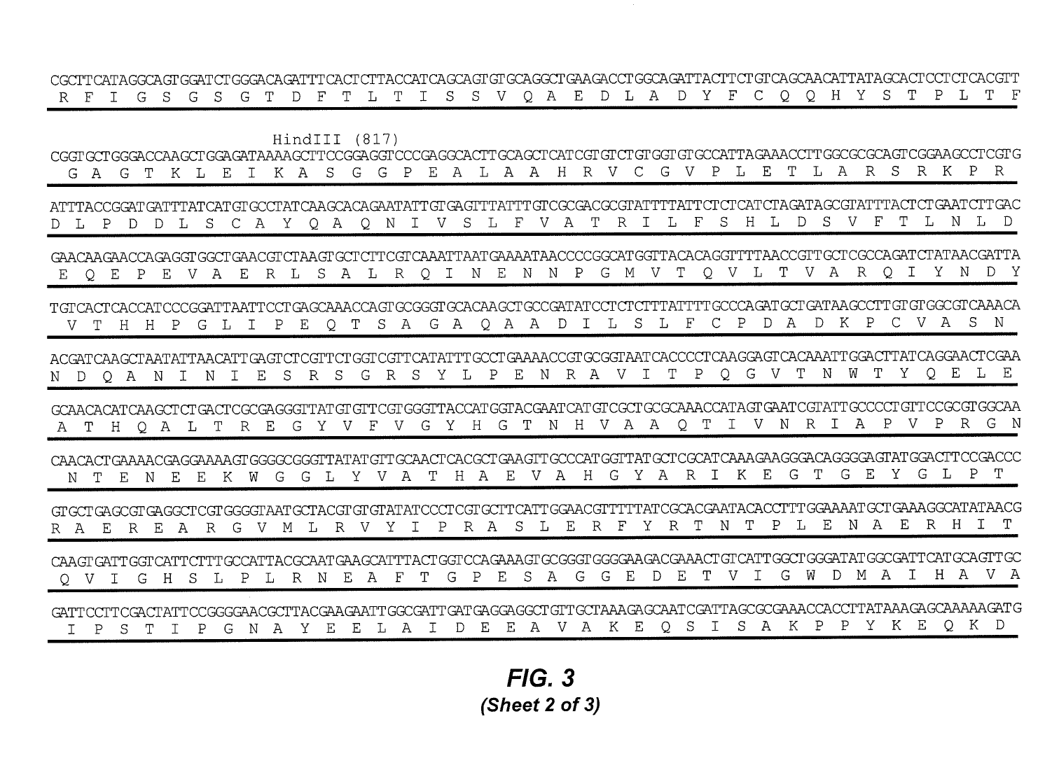

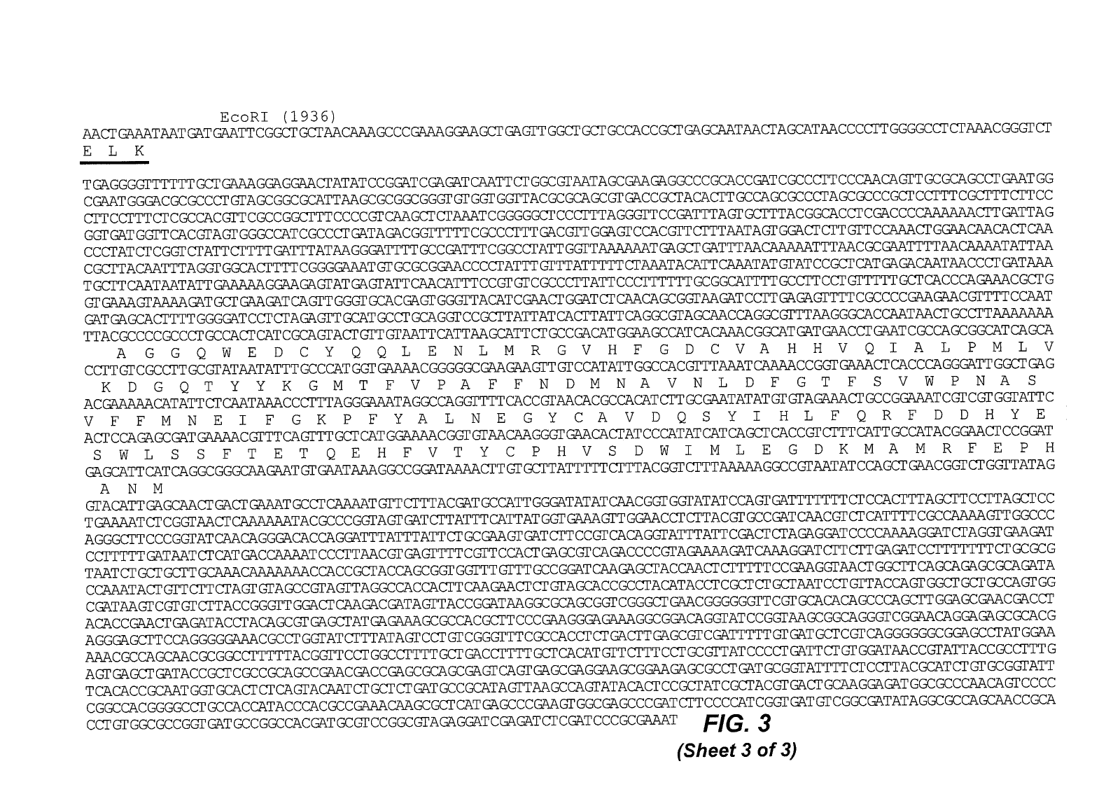

To make the single chain immunotoxin, referred to herein as HB21scFv-CET40 (and sometimes to as HB21-CET40), a cDNA encoding the Fv portion of the HB21 antibody recognizing the human transferrin receptor, a receptor known to be efficiently internalized (Batra et al., 1991, Mol Cell Biol 11:2200-5) was fused in frame with a synthetic gene encoding domains II, Ib, and III of cholera exotoxin (here called CET). CET differs from the toxin named ‘cholix toxin’ (GenBank accession number AY876053; Jorgensen et al., 2008, J Biol Chem 283:10671-8) by 14 amino acid residues (see FIG. 9C). The synthetic CET gene encoded amino acids 270-634 of CET (the annotated DNA and protein sequences are provided in FIG. 9A). The synthetic nucleic acid sequences encoding CET40 are shown in SEQ ID NOS:3 and 33 and in FIG. 3. Amino acid residues 270-634 of CET encompass domains II, III, and a small subdomain, Ib. For simplicity, domain Ib is not routinely mentioned herein. The CET sequence was derived from the sequenced genome of Vibrio cholerae strain 1587 (GenBank accession number for CET is ZP_{—}01950668) and differs from cholix toxin (CT) in domains II and III by ten amino acids (FIG. 9C). FIG. 9B shows a clustal X sequence alignment of domains II and III of cholix toxin (Vibrio cholerae strain TP (Purdy et al., 2005, J Bacteriol 187:2992-3001), CET (Vibrio cholerae strain 1587) and PE40.

Key features of each toxin include a consensus furin cleavage sequence (with strong conservation on the N-terminal site of the scissile bond and weak conservation on the C-terminal side), a conserved glutamic acid marking the NAD binding pocket and a C-terminal a KDEL (SEQ ID NO:4)-like sequence followed by a terminal lysine. Also the four half cysteines are completely conserved as are several stretches of residues within domain III (from residues 187-336 in FIG. 9B). Half cysteines refers to presumed disulfide bonds without committing to specific bonding pairs.

The synthetic gene fragment (sequence provided in SEQ ID NOS:3 and 33 and as part of SEQ ID NO:21 in FIG. 9A) encoding putative domains II and III of Cholera exotoxin was produced (at Blue Heron Biotechnology) with HindIII and EcoRI restriction sites flanking the gene, and provided as a pUC19 plasmid. Vector DNA was digested with the appropriate restriction endonucleases, separated via 0.9% agarose gel electrophoresis and the fragments gel purified using a Qiaquick gel extraction kit. Ligations of DNA fragments were performed using an approximate (3:1) insert to vector molar ratio with T4 DNA ligase (New England Biolabs) in 1× ligation buffer, at 37° C. for 1.5 hrs or 16 hrs at 16° C. Clones were screened by diagnostic restriction digests and positive clones confirmed by DNA sequencing (Johns Hopkins Sequencing Facility, Baltimore, Md.). The CET40 encoding DNA fragment was then cloned into a vector backbone having the coding region of the single chain Fv sequence binding to the transferrin receptor to arrive at pHB21+CET40 (see FIG. 3).

### Example 3

### Construction of PE40 Immunotoxin

For comparison, HB21 was separately cloned into a vector containing the cDNA encoding the 40 kD cytotoxic fragment of Pseudomonas exotoxin A known as “PE40” to create the recombinant immunotoxin HB21-PE40. A pBR322-based expression vector, pRB2506, encoding HB21scFv-PE40 was provided by Richard Beers and Ira Pastan.

Original Pseudomonas exotoxin-based immunotoxins were constructed with domains II and III together with the subdomain termed domain Ib and were called PE40 (Chaudhary et al., 1989, Nature 339:394-7). However, recent iterations have been made with a deletion of a portion of domain Ib (amino acids 365-380) and are termed PE38 (Brinkmann et al., 1991, Proc Natl Acad Sci USA 88:8616-20).

### Example 4

### Expression of a CET40 Immunotoxin

Briefly, expression of HB21scFv-CET40 was driven by a T7 promoter and accomplished via growth in ‘autoinduction media’ under Cm selection. Specifically, the backbone of the pBR322 expression vector has an inducible T7 promoter and carries a gene encoding chloramphenicol resistance. Expression of the single chain immunotoxin was carried out in BL21-Star (DE3) E. coli cells (Invitrogen) grown at 37° C. in baffled Fernbach flasks at 275 rpm. Cells were grown in Superbroth (KD Medical) supplemented with chloramphenicol at 25 μg/ml (Sigma) and ‘Overnight Express’ additives (Novagen). This medium was inoculated with freshly transformed cells and grown overnight (˜17 hrs). Final culture OD₆₀₀ were ˜5-6. Cells were harvested by centrifugation at 4000×g for 10 minutes in a Sorvall 3B centrifuge. Cell pellets were stored frozen at −80° C. or processed for protein purification.

After an overnight culture, the insoluble protein was recovered in inclusion bodies and purified as described herein (Buchner et al., 1992, Biotechnology (NY) 10:682-5; Buchner et al., 1992, Anal Biochem 205:263-70). Briefly, inclusion bodies were solubilized with 6M guanidine and a reducing agent, refolded into a redox shuffling buffer and purified using anion exchange and gel filtration chromatography. An SDS-PAGE analysis of gel filtration fractions revealed that ˜20% of HB21scFv-CET40 eluted as a monomer (FIG. 10). Fractions 28 and 29 were used for experiments described herein.

### Example 5

### Contacting Cells Expressing Transferrin Receptor with the Immunotoxin HB21-PE40

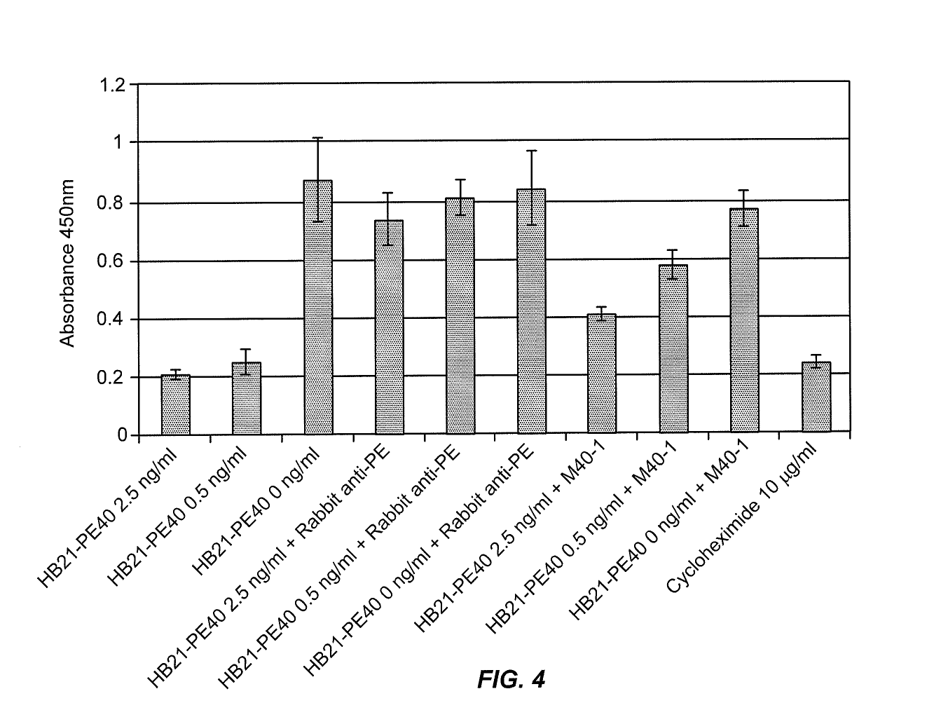

FIG. 4 shows the results of studies employing HB21-PE40 at concentrations of 2.5 ng/ml and 0.5 ng/ml. Immunotoxin and antibody were pre-mixed for 30 min at room temperature and the mixture added to DLD-1 colon cancer cells. Cells were incubated for 48 hrs. Cells were assessed for viability using a WST-1 cell proliferation assay. Both concentrations of HB21-PE40 stopped the growth and proliferation of the transferrin receptor-expressing colon cancer cells to the same degree as the protein synthesis inhibitor cycloheximide (compare first and second bars on the left with last bar on the right side of FIG. 4). When the cells were exposed to the same concentrations of the same immunotoxin but in the presence of rabbit anti-PE polyclonal antibodies (as shown in the fourth and fifth bars from the left) the cells grew and proliferated to the same extent as cells in medium without the immunotoxin (third bar from the left: “0 ng/ml” of immunotoxin) or in medium without the immunotoxin but with the anti-PE polyclonal antibodies (5th bar from the left). The Figure also shows that an anti-PE monoclonal antibody, called “M40-1” (Ogata et al., Infect Immun., 59(1):407-14 (1991)), provided less protection to cells exposed to 2.5 ng/ml and 0.5 ng/ml of the immunotoxin than did the anti-PE polyclonal antibody (compare 4th and 5th bars from the left with 7th and 8th bars from the left of FIG. 4).

### Example 6

### Contacting Cells Expressing Transferrin Receptor with the Immunotoxin HB21-CET40

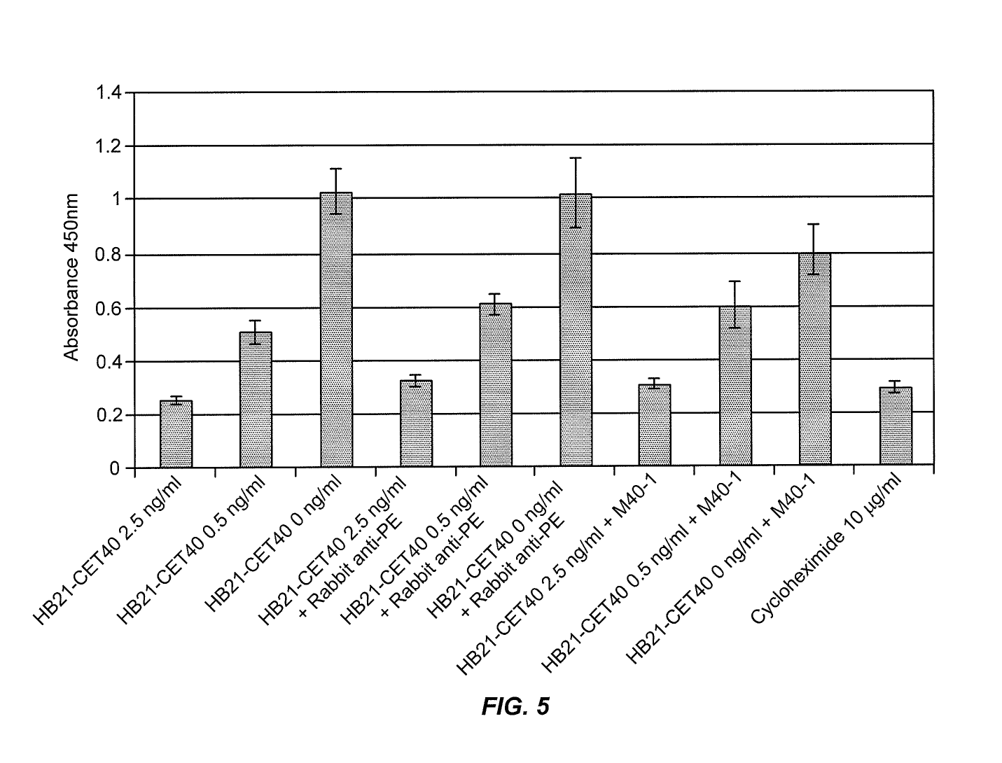

FIG. 5 shows the results of identical experiments as described in Example 3, using, in place of the PE-based immunotoxin, the HB21-CET40 recombinant immunotoxin, in which domain Ia of CT has been deleted. As shown in FIG. 5, the effect of the immunotoxin is essentially the same in the presence or the absence of either the anti-PE polyclonal and monoclonal antibodies, showing that the CT was not neutralized by either set of antibodies.

### Example 7

### Contacting Cells Expressing Transferrin Receptor with the Immunotoxin HB21-PE40 in the Presence or Absence of an Anti-PE Antibody

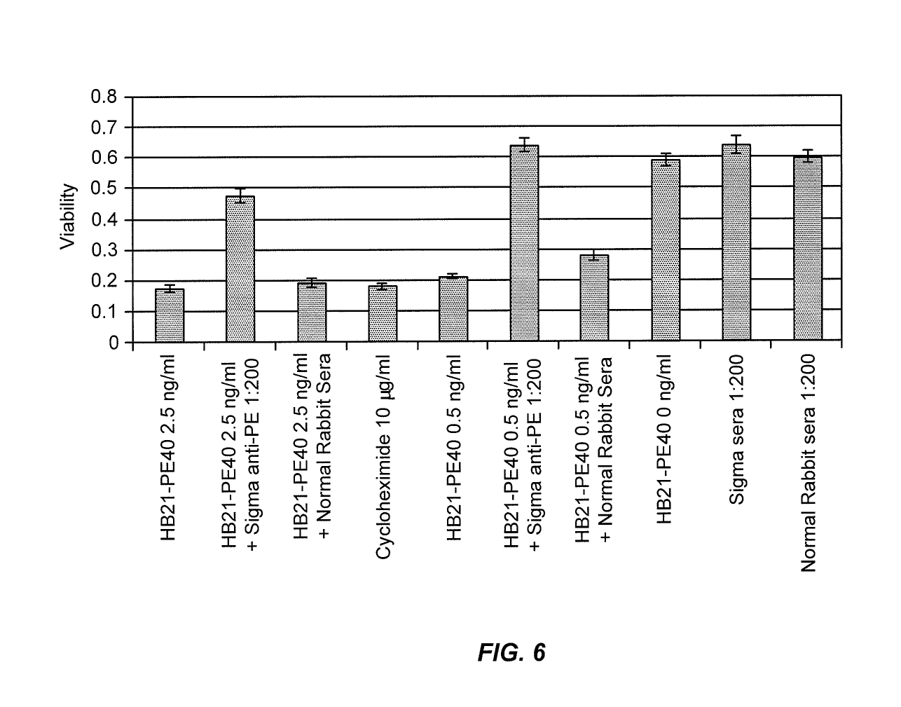

Similarly, FIG. 6 shows the affect of the HB21-PE40 immunotoxin on the growth and proliferation of transferrin receptor-expressing cells in the presence or absence of a commercially available rabbit anti-PE polyclonal antibody, sold as a whole serum (Cat. No. P2318, Sigma-Aldrich, St. Louis, Mo.). As shown in the second bar of FIG. 6, the anti-PE antibody serum provides significant protection to the cells against the presence of the immunotoxin (compare 1st and 2nd bars from the left of FIG. 6), while serum from un-immunized rabbits (“normal rabbit sera”) provides no such protection from the immunotoxin (see third bar).

### Example 8

### Contacting Cells Expressing Transferrin Receptor with the Immunotoxin HB21-CET40 in the Presence or Absence of an Anti-PE Antibody

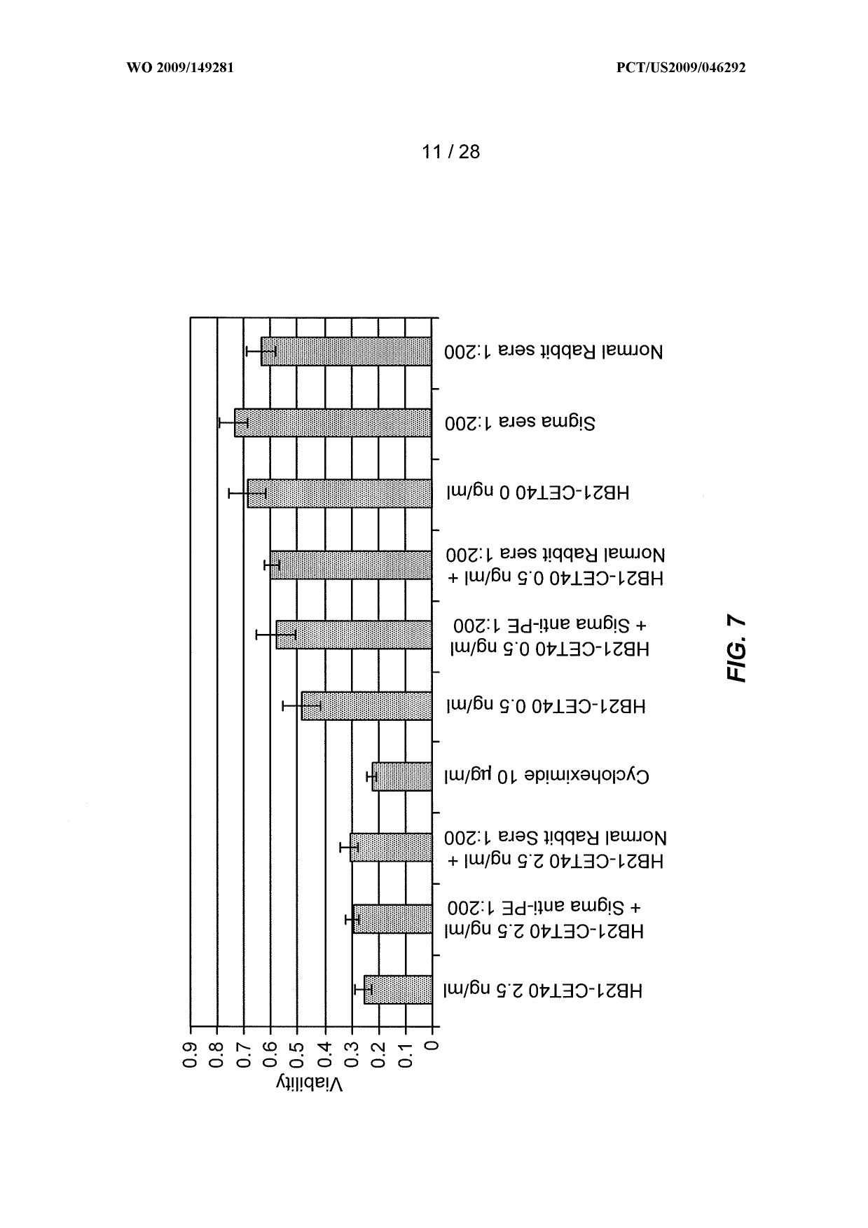

FIG. 7 shows the same experiment as described in Example 6, but using the CET40-based immunotoxin. As shown in FIG. 7, neither the anti-PE rabbit sera nor the normal rabbit sera provide the cells any protection from the HB21-CET40 immunotoxin (FIG. 7, compare first, second, and third bars from the left with the fourth bar, which is the cycloheximide positive control).

### Example 9

### Anti-PE Antibodies React with HB21-PE40, but not with HB21-CET40

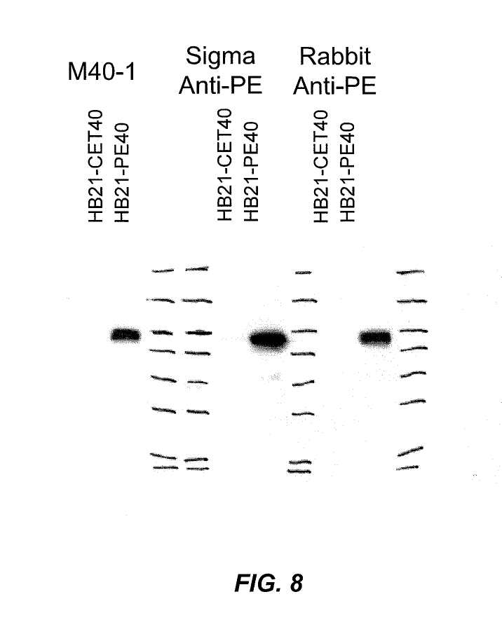

FIG. 8 shows the results of Western blots conducted with approximately 25 ng of purified immunotoxin. As can be seen in FIG. 8, the anti-PE antibodies reacted with HB21scFv-PE40 (“HB21-PE40”) but not with HB21scFv-CET40 (“HB21-CET40”).

### Example 10

### Cytotoxic Activity of the Immunotoxin HB21scFv-CET40

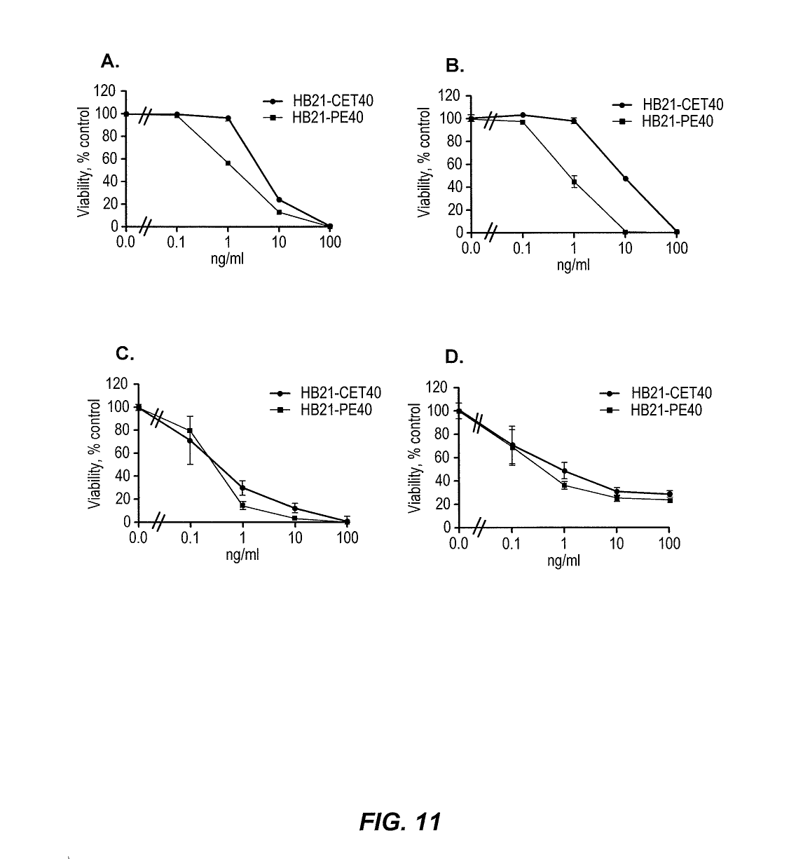

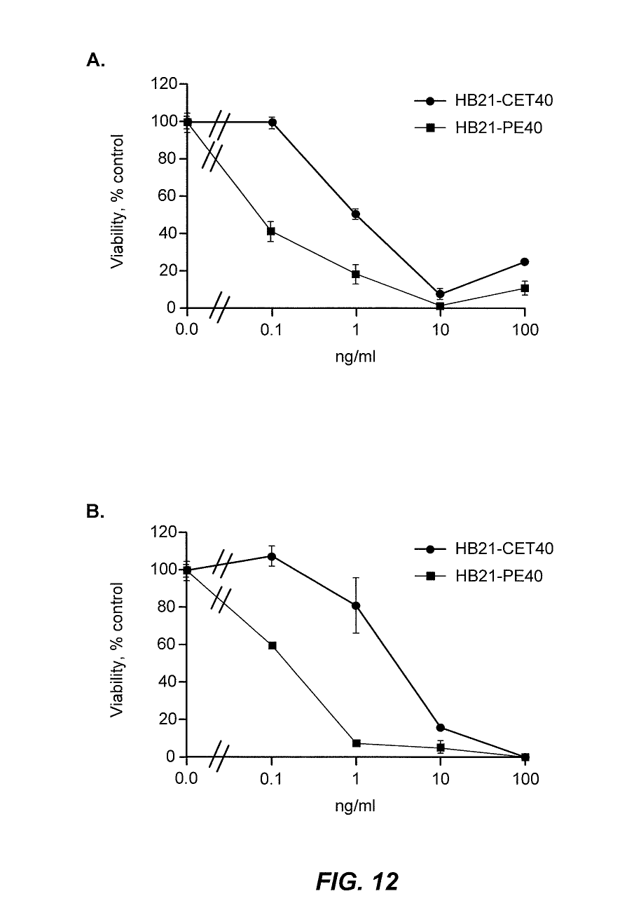

HB21scFv-CET40 was assayed for cell-killing activity against several cell lines and compared directly with HB21scFv-PE40. The following lines of various tissue origins were tested: DLD-1, colon; A549, lung; KB3-1, epidermoid; 293TT, kidney; Raji, B-cell; and HUT102, T-cell. In all cells tested, HB21scFv-CET40 was equipotent to ten times less active when compared to HB21 scFv-PE40 (FIG. 11A-D and FIG. 12 A,B). Generally, the adherent epithelial cancer cell lines (FIG. 11A-D and FIG. 12 A,B) were ˜5-fold more sensitive to the PE40 immunotoxin while lymphoid cancers (FIG. 11C, D) exhibited equal sensitivity the PE40/CET40 immunotoxins. Thus, it can be concluded that CET40 is a potent cytotoxic molecule that can be targeted using an antibody Fv to an antigen on the surface of a cancer cell.

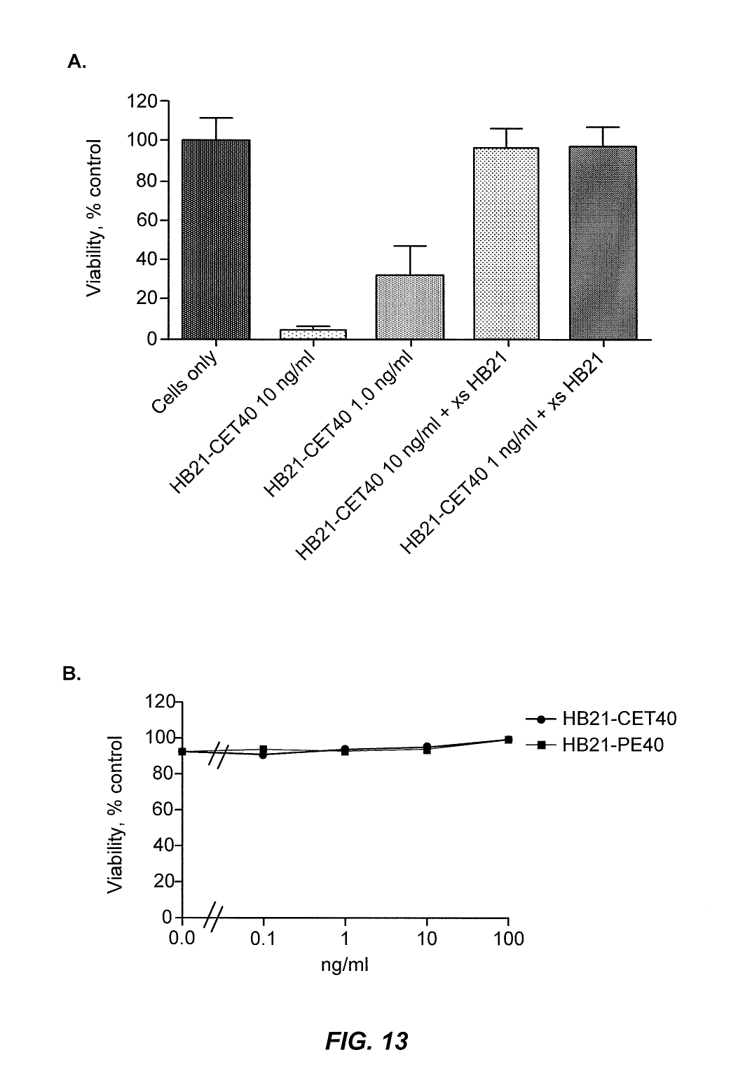

Two specific controls were included in this set of experiments: To confirm specificity of activity, excess HB21 antibody was used in competition experiments to block the human transferrin receptor (huTFR) and reduce immunotoxin activity (FIG. 13A). The pretreatment of cells with 10 ug/ml of HB21 completely abrogated the toxicity seen with either 1 or 10 ng/ml of HB21scFv-CET40.

Because there is no cross-reactivity of the HB21 antibody with the murine TFR, as another specificity control, HB21scFv-CET40 was added to the mouse L929 cell line to assess any non-specific toxicity that might be contributed by CET40 (FIG. 13B). No reduction in viability was noted in concentrations up to 100 ng/ml. These latter two control experiments confirm that cell binding of the immunotoxin HB21scFv-CET40 is via the targeting antibody Fv and not mediated by CET40 residues. Thus, CET40 can be used to construct potent and antigen-specific recombinant immunotoxins other than HB21scFv-CET40.

### Example 11

### Cytotoxic Activity of the Immunotoxin HB21scFv-CET40

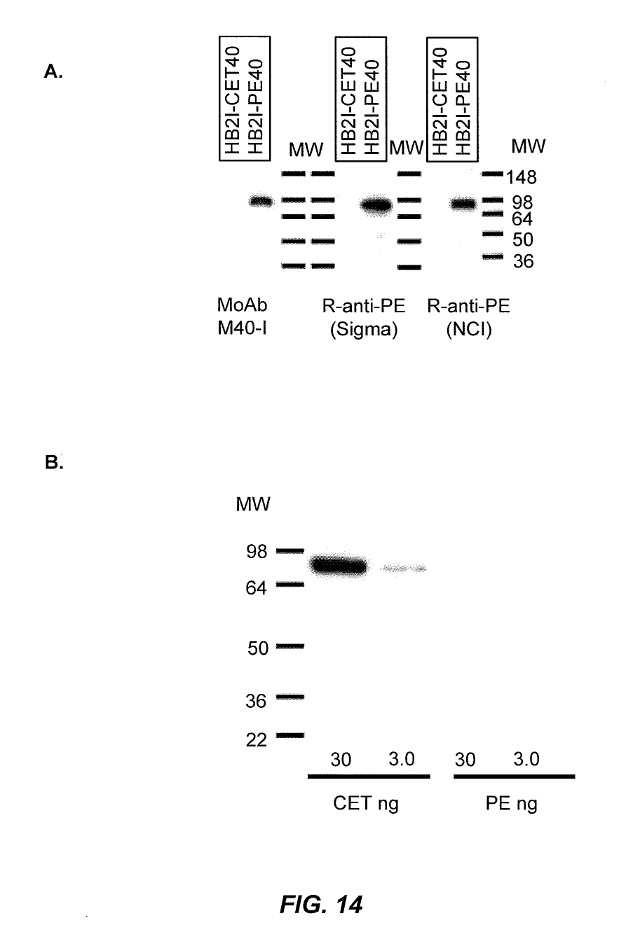

Because of the close structural and sequence similarity of PE40 to CET40, preparations of CET40 were probed with anti-PE antibodies looking for evidence of cross-reactivity. Surprisingly, it was found that two distinct rabbit anti-PE polyclonal antibody preparations and one monoclonal antibody, each strongly reactive for HB21scFv-PE40, were unreactive for HB21scFv-CET40 (FIG. 14 A). In an attempt to conduct the reciprocal experiment, rabbit antibodies to CET40 were produced by hyperimmunizing with a preparation of HB21scFv-CET40E581A, an enzymatically inactive form of CET40. Because this antibody preparation contained antibodies to both CET40 and the HB21scFv (data not shown) Western blot analysis of PE40 immunotoxins could not be used to assess cross-reactivity. Instead, reactivity was assessed using full length CET and PE. As shown in FIG. 14B, the rabbit anti-CET40 preparation reacted with CET but not PE, providing further evidence that the two toxins are immunologically distinct.

### Example 12

### Neutralization Assays

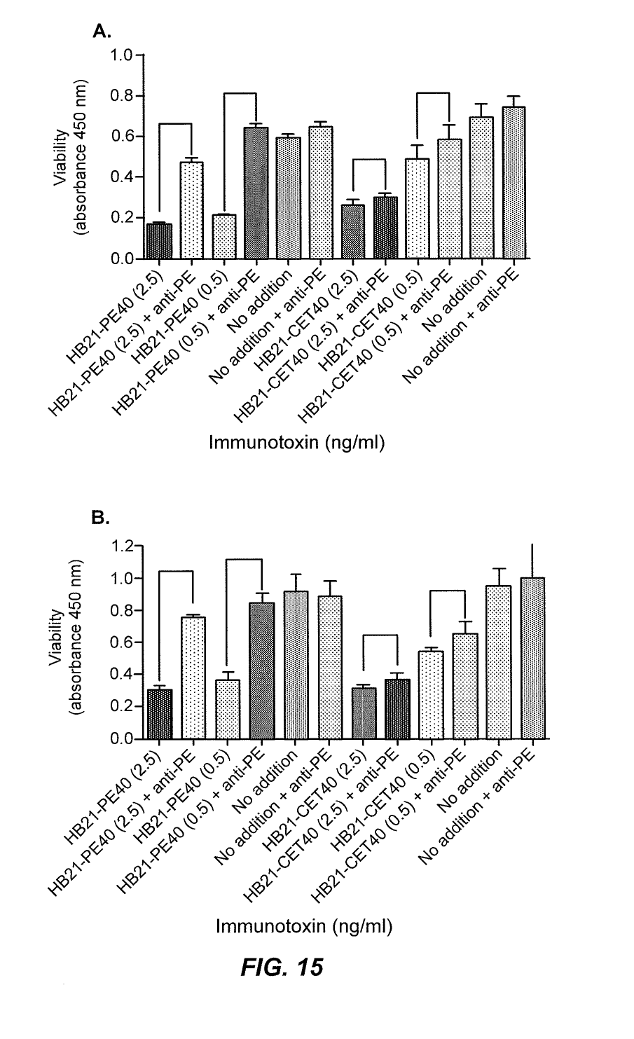

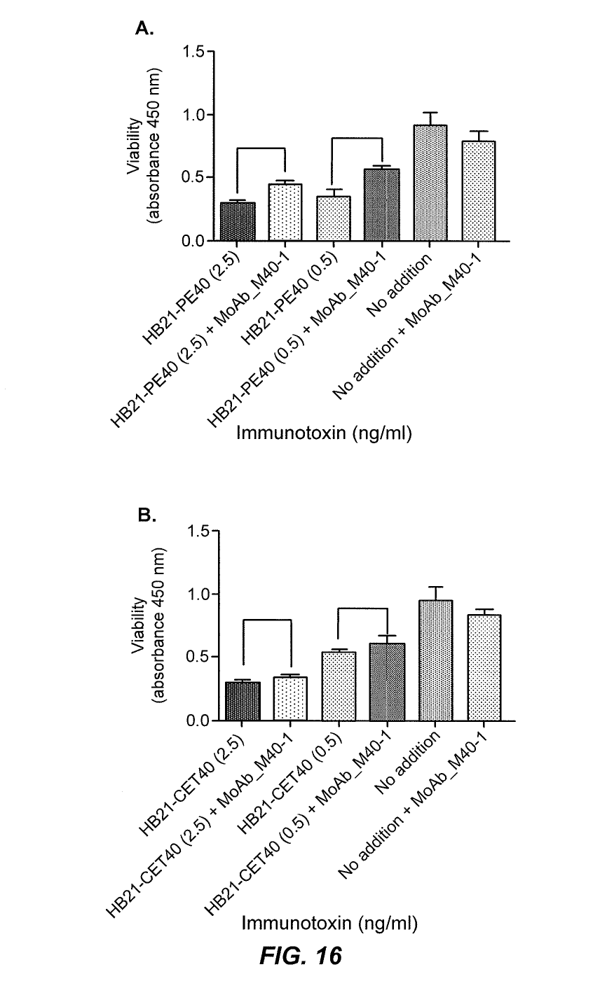

Because conformational epitopes may not be recognized in Western blots, a neutralization assay was performed where antibodies and immunotoxins were mixed in solution and then added to cells. To assess neutralization activity, HB21scFv-PE40 and HB21scFv-CET40 at either 5 or 1 ng/ml were each mixed with either 20 μg/ml of Rabbit anti-PE IgG (a lab reagent applicants raised to formaldehyde-treated full length PE) or 20 μg/ml of the monoclonal antibody M40-1 (Batra et al., 1991, Mol Cell Biol 11:2200-5) or with a 1:100 dilution of commercial antisera to PE available from Sigma-Aldrich. Mixtures of antibody and immunotoxin were incubated for 1 hr at room temp after which aliquots were added to cells. Addition to cells resulted in a 2-fold dilution of all reagents, so that immunotoxins at 2.5 and 0.5 ng/ml were incubated with target cells in the continued presence of the test antibodies. The rabbit anti-PE antibodies neutralized completely the PE40 immunotoxin but not the CET40 version (see lines on FIGS. 15 A-B indicating comparisons at both 2.5 and 0.5 ng/ml of immunotoxin). The mouse monoclonal antibody showed modest neutralizing activity against the PE40 immunotoxin and none against the CET40 immunotoxin (FIG. 16). These data showed that the two toxins do not share a common epitope that is recognized by neutralizing antibodies.

### Example 13

### Activity of Human Anti-PE38 Sera

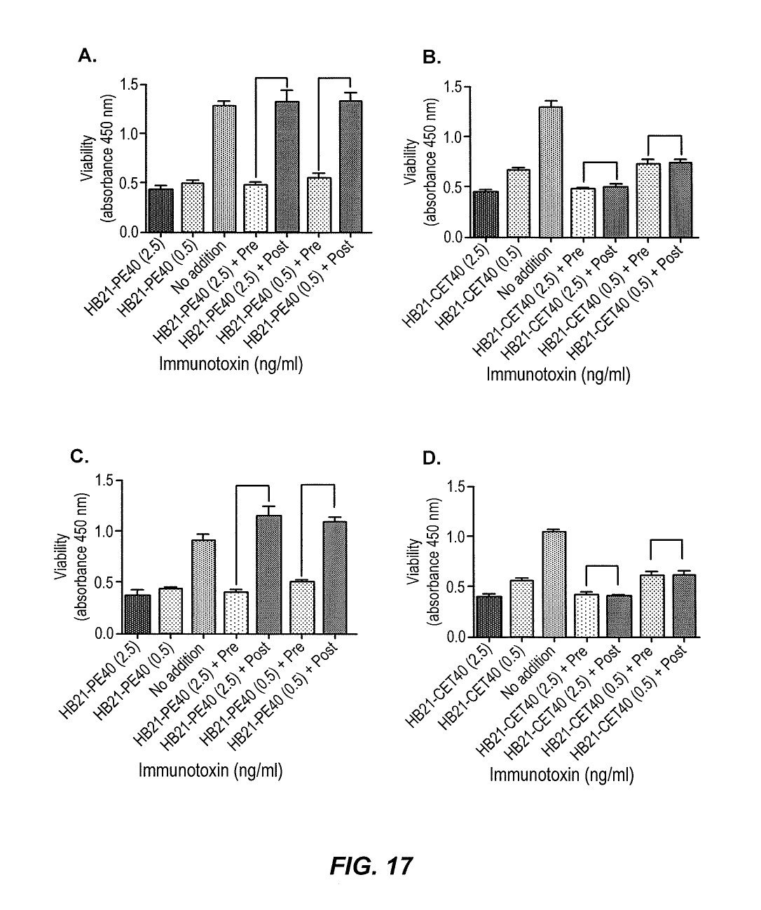

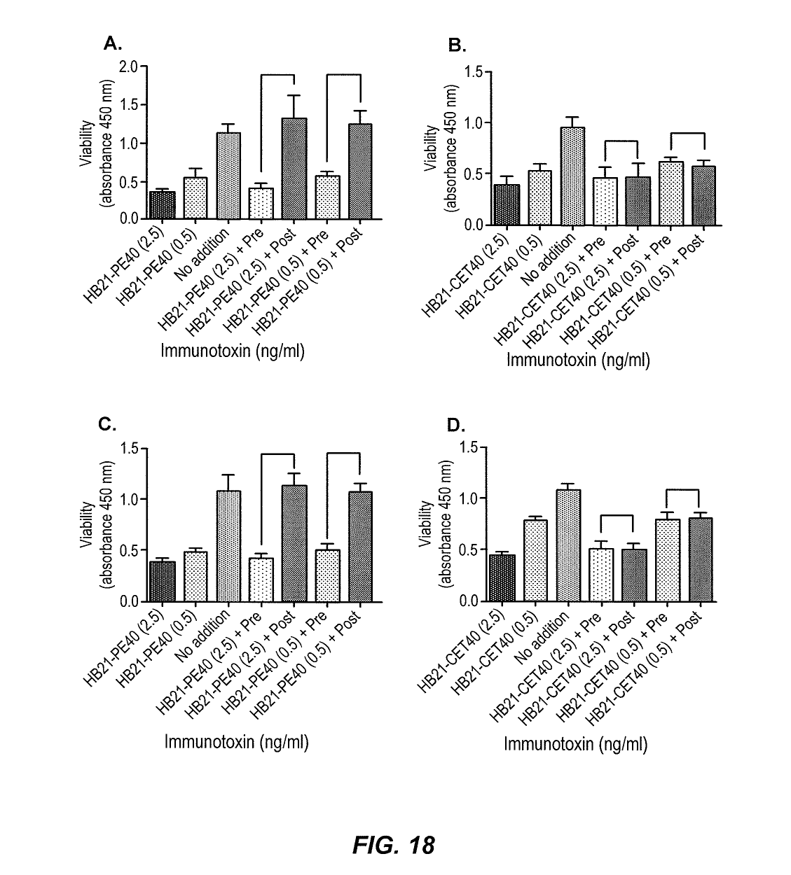

The administration of PE38 immunotoxins to non-immunosuppressed cancer patients usually (>90% of the time) results in the formation of neutralizing antibodies (Hassdan et al., 2007, Clin Cancer Res 13:5144-9). Human anti-immunotoxin response is most often directed to the toxin portion of the immunotoxin (Posey et al., 2002, Clin Cancer Res 8:3092-9; Kreitman et al., 2000, J Clin Oncol 18:1622-36). As described herein, when compared at the level of primary sequence, domains II and III of PE and CET are 36% identical. It was therefore of some interest to learn whether conserved residues included those that generated human neutralizing antibodies. To examine this, sera from four patients that had developed neutralizing anti-PE38 antibodies were each evaluated for neutralizing activity to HB21scFv-CET40. These patients had each been treated with SS1P, a PE38 immunotoxin directed to the surface differentiation antigen mesothelin, and had developed a neutralizing antibody response to PE38. Therefore, a pre- and post-treatment sample from each patient was tested and neutralization activity against 5 ng/ml and 1 ng/ml of HB21scFv-PE40 and HB21scFv-CET40 was assessed (see red bars on FIG. 17 A-D and FIG. 18 A-D). Each serum sample gave essentially the same result: full neutralization of HB21scFv-PE40 at 5 and 1 ng/ml by the post treatment sample but no neutralization with the pretreatment sample. In contrast, there was no neutralization of HB21scFv-CET40 by either the pre or post treatment sera, confirming that, in (4-of-4) humans, PE38 was immunologically distinct from CET40 with respect to the production of neutralizing antibodies.

### Example 14

### Immunotoxins

Immunotoxins, antibody-toxin fusion proteins, are under development as cancer therapeutics. In early clinical trials, immunotoxins constructed with domains II and III of Pseudomonas exotoxin (termed PE38), have produced a high rate of complete remissions in hairy cell leukemia and objective responses in other malignancies. Cholera exotoxin (also known as cholix toxin) has a very similar three-dimensional structure to Pseudomonas exotoxin (PE) and when domains II and III of each are compared at the primary sequence level, they are 36% identical and 50% similar.

Herein the construction and activity of immunotoxins made with domains II and III of cholera exotoxin (CET40) is described. In cell viability assays, CET40 immunotoxins were equipotent to 10-fold less active compared to PE-based immunotoxins made with the same single chain Fv.

The reason or reasons for reduced cell killing by CET40 compared with PE40 have not yet been elucidated. However, differences in three key sequences that are known to be important in PE-based immunotoxins are being considered. Both toxins have a consensus furin cleavage sequence, a glutamic acid for binding NAD and a KDEL (SEQ ID NO:4)-like sequence at the end of the molecule (see FIG. 9B). The furin recognition site has a P1 arginine and P4 arginine in cholix, CET and PE. Also present is a P6 arginine that represents an extended furin cleavage sequence (FIG. 9B). The P2 residue is a proline, which is not usual for substrates of furin but apparently is functional at this location (Matthews et al., 1994, Protein Sci 3:1197-205). While residues P1-6 appear well conserved, P′1-7 residues are not (FIG. 9B). A key tryptophan (Zdanovsky et al., 1993, J Biol Chem 268:21791-9) at the P′2 position of PE (residue 281 in native PE) is replaced with a leucine while the P′ 1 glycine of PE is replaced with an aspartic acid in CET. These residues and others in the vicinity may contribute to an altered efficiency of translocation to the cytosol.

NAD binding relies on a glutamic acid in all three toxins. However in cholix and CET there are two negatively charged residues immediately preceding this residue. In PE there is an arginine and leucine instead.

Finally, PE is known to require a KDEL (SEQ ID NO:4)-like sequence for cytotoxic activity presumably because retrograde transport to the ER is essential for toxicity. Both cholix and CET terminate in the sequence ‘KDELK’ (SEQ ID NO:8) while PE terminates with ‘REDLK’ (SEQ ID NO:5). At this time it is not known if these variants behave equally well for retrieval to the ER and whether they influence translocation efficiency to the cytosol. In sum there are several differences between the two toxins and these contribute to altered efficiencies in different cell types. One approach to study this is to make hybrid toxin molecules where domains or domain segments are swapped and activities compared.

A major limitation of toxin-based immunotoxins is the development of neutralizing antibodies to the toxin portion of the immunotoxin. Because of structure and sequence similarities, it was important to evaluate CET40 immunotoxins for the presence of PE-related epitopes. In Western blots, 3-of-3 anti-PE antibody preparations failed to react substantially with CET40 immunotoxins. More importantly, in neutralization studies neither these antibodies nor those from patients with neutralizing titers to PE38, neutralized CET40-immunotoxins. It is proposed herein that the use of modular components such as antibody Fvs and toxin domains will allow a greater flexibility in how these agents are designed and deployed including the sequential administration of a second immunotoxin after patients have developed neutralizing antibodies to the first.

To construct an immunotoxin of the present invention, a synthetic gene encoding amino acids 270 to 634 of cholera exotoxin (CET) was combined with the single chain Fv antibody (HB21scFv) directed to the human transferrin receptor. HB21scFv-CET40 was potently toxic for a number of human cancer cell lines. Despite a high level of structural and sequence similarity between PE40 and CET40, anti-PE antibodies did not recognize or neutralize the CET40 immunotoxin. Thus, it is now possible to develop a distinct anti-cancer therapeutic platform centered on CET-based immunotoxins that potentially can be administered as a first line therapeutic agent or to individuals with prior exposure to PE-based immunotoxins.

What is claimed is:

1. An isolated toxin comprising:
(i) a domain III of cholera exotoxin (CET) having an amino-terminal sequence and a carboxy terminal sequence, and at least 65% sequence identity to an amino acid sequence of SEQ ID NO:36.

2. The isolated toxin according to claim 1, wherein said domain III is selected from a group consisting of a CET domain III having greater than about 85% sequence identity to an amino acid sequence of SEQ ID NO:36, a CET domain III having greater than about 90% sequence identity to an amino acid sequence of SEQ ID NO:36, a CET domain III having greater than about 91% sequence identity to an amino acid sequence of SEQ ID NO:36, a CET domain III having greater than about 92% sequence identity to an amino acid sequence of SEQ ID NO:36, a CET domain III having greater than about 93% sequence identity to an amino acid sequence of SEQ ID NO:36, a CET domain III having greater than about 94% sequence identity to an amino acid sequence of SEQ ID NO:36, a CET domain III having greater than about 95% sequence identity to an amino acid sequence of SEQ ID NO:36, a CET domain III having greater than about 96% sequence identity to an amino acid sequence of SEQ ID NO:36, a CET domain III having greater than about 97% sequence e identity to an amino acid sequence of SEQ ID NO:36, a CET domain III having greater than about 98% sequence identity to an amino acid sequence of SEQ ID NO:36, and a CET domain III having greater than about 99% sequence identity to an amino acid sequence of SEQ ID NO:36.

3. The isolated toxin according to claim 1, further comprising at least one of amino acid residues 253E, 283R, 352A, or 359Q of SEQ ID NO:2.

4. The isolated toxin according to claim 1, further comprising:
(ii) a furin cleavage sequence having an amino-terminal sequence and a carboxy terminal sequence;
wherein said carboxy terminal sequence of said furin cleavage sequence is fused to said amino-terminal sequence of said CET domain III.

5. The isolated toxin according to claim 4, wherein said furin cleavage sequence is selected from the group consisting of a CET furin cleavage sequence and a Pseudomonas exotoxin A furin cleavage sequence.

6. The isolated toxin according to claim 1, wherein said domain III comprises a NAD binding site.

7. The isolated toxin according to claim 6, wherein said NAD binding site is a CET or PE NAD binding site.

8. The isolated toxin according to claim 1, wherein said domain III of CET comprises an amino acid sequence of SEQ ID NO:36 or a conservatively modified fragment thereof, wherein said isolated toxin has cytotoxic activity.

9. The isolated toxin according to claim 1, wherein said toxin is a CET40 having an amino acid sequence of at least 85% identity to SEQ ID NO:24.

10. The isolated toxin according to claim 9, further comprising at least one of amino acid residues selected from the group consisting of 26P, 73A, 76Q, 107I, 131P, 254E, 284R, 353A, and 360Q of SEQ ID NO:24.

11. The isolated toxin according to claim 1, wherein said isolated toxin is a CET40 having an amino acid sequence of SEQ ID NO:24.

12. The isolated toxin according to claim 1, wherein said carboxyl terminal sequence of said CET domain III is REDLK (SEQ ID NO:5).

13. The isolated toxin according to claim 1, wherein the CET domain III comprises amino acid residues corresponding to amino acid residues 293 and 294 of SEQ ID NO:1 which are selected from the group consisting of: D293G-L294W, D293D-L294W, and D293 G-L294L.

14. The isolated toxin according to claim 1, further comprising:
(iii) a targeting moiety which specifically binds to one or more cell surface markers;
wherein said targeting moiety is fused in frame to said toxin.

15. The isolated toxin according to claim 14, wherein said cell surface marker is a cell surface receptor.

16. The isolated toxin according to claim 14, wherein said targeting moiety is an antibody or antibody fragment specifically binding to said one or more cell surface markers.

17. The isolated toxin according to claim 16, wherein said antibody or antibody fragment specifically binds to a cell surface marker selected from the group consisting of transferrin receptor, EGF receptor, CD19, CD22, CD25, CD31, CD79, mesothelin, and cadherin.

18. The isolated toxin according to claim 16, wherein said antibody fragment is selected from the group consisting of a Fab, a Fab′, a F(ab′)2, a scFv, a Fv fragment, a helix-stabilized antibody, a diabody, a disulfide stabilized antibody, and a domain antibody.

19. The isolated toxin according to claim 18, wherein said antibody fragment is a scFv.

20. The isolated toxin according to claim 19, wherein said isolated toxin specifically binds to a transferrin receptor.

21. The isolated toxin according to claim 20, wherein said isolated toxin comprises an amino acid sequence of SEQ ID NO:19.

22. The isolated toxin according to claim 14, wherein said targeting moiety is a ligand specifically binding to said one or more cell surface markers.

23. The isolated toxin according to claim 22, wherein said cell surface marker is a cell surface receptor.

24. A method for inhibiting the growth of a population of cells bearing one or more cell surface markers, comprising the step of:
(a) contacting said population of cells with a first isolated toxin according to claim 1; thereby inhibiting the growth of said population of cells.

25. The method according to claim 24, further comprising the step of:
(b) contacting said population of cells with a second isolated toxin comprising:
(i) a Pseudomonas exotoxin A (PE) toxin, and
(ii) a targeting moiety which specifically binds at least one of said surface markers.

26. The method according to claim 25, wherein step (b) is perfoil ied prior to step (a).

27. The method according to claim 25, wherein said first isolated toxin is administered to said population of cells about three weeks after administration of said second isolated toxin to said population of cells.

28. The method according to claim 25, wherein said first isolated toxin is administered to said population of cells within about one month of administration of said second isolated toxin to said population of cells.

29. The method according to claim 25, wherein said first isolated toxin is administered to said population of cells within about two months of administration of said second isolated toxin to said population of cells.

30. The method according to claim 25, wherein said targeting moiety of said first and said second isolated toxins specifically bind to the same cell surface marker.

31. The method according to claim 30, wherein said targeting moiety of said first and said second isolated toxins is the same.

32. The method according to claim 24, wherein said targeting moiety is an antibody or antibody fragment specifically binding to said one or more cell surface markers.

33. The method according to claim 32, wherein said antibody fragment is selected from the group consisting of a Fab, a Fab′, a F(ab′)2, a scFv, a Fv fragment, a helix-stabilized antibody, a diabody, a disulfide stabilized antibody, and a domain antibody.

34. The method according to claim 33, wherein said antibody fragment is a scFv.

35. The method according to claim 25, wherein said PE is a PE40 comprising an amino acid sequence of SEQ ID NO:25 or a conservatively modified cytotoxic variant thereof.

36. The method according to claim 24, wherein said first isolated toxin comprises an amino acid sequence of SEQ ID NO:2 or a conservatively modified cytotoxic variant thereof.

37. The method according to claim 24, wherein said first isolated toxin comprises an amino acid sequence of SEQ ID NO:24 or a conservatively modified cytotoxic variant thereof.

38. The method according to claim 24, wherein said first isolated toxin comprises a NAD binding site of PE.

39. The method according to claim 24, wherein the C-terminal amino acid sequence KDELK (SEQ ID NO:8) of the CET domain III is replaced by the amino acid sequence REDLK (SEQ ID NO:5).

40. The method according to claim 24, wherein, said population of cells are mammalian cells.

41. The method according to claim 40, wherein said mammalian cells are human cells.

42. The method according to claim 41, wherein said human cells are disease cells or malignant cells.

43. The method according to claim 42, wherein said malignant cells are cancer cells selected from the group consisting of neuroblastoma, intestine carcinoma, rectum carcinoma, colon carcinoma, familiary adenomatous polyposis carcinoma, hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors, glioblastoma, astrocytoma, meningioma, medulloblastoma, peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroids melanoma, seminoma, rhabdomyosarcoma, craniopharyngeoma, osteosarcoma, chondrosarcoma, myosarcome, liposarcoma, fibrosarcoma, Ewing sarcoma, and plasmocytoma.

44. The method according to claim 24, wherein said cell surface marker is a cell surface receptor.

45. The isolated toxin according to claim 24, wherein said cell surface marker is selected from the group consisting of transferrin receptor, EGF receptor, CD 19, CD22, CD25, CD31, CD79, mesothelin, and cadherin

46. The method according to claim 45, wherein at least one of said cell surface marker is mesothelin.

47. The method according to claim 45, wherein at least one of said cell surface marker is CD22.

48. A method of providing therapy for a mammal having developed neutralizing antibodies to Pseudomonas exotoxin A, comprising the steps of:
(a) selecting a mammal having developed neutralizing antibodies to Pseudomonas exotoxin A;
(b) administering to said mammal an isolated toxin according to claim 1.

49. A method of providing therapy for a mammal having developed a disease caused by the presence of cells which bearing one or more cell surface markers, comprising the steps of:
(a) administering to said mammal an isolated toxin according to claim 1; and
(b) administering to said mammal an isolated toxin comprising:
(i) a targeting moiety which specifically binds to at least one surface marker on said cells; and
(ii) a Pseudomonas exotoxin A toxin.

50. The method according to claim 49, wherein step (a) is performed prior to step (b).

51. The method according to claim 49, wherein step (b) is performed prior to step (a).


SEQUENCE LISTING (36 sequences)

SEQ ID NO: 1 - protein, 634 aa, Vibrio cholerae
  mature cholera exotoxin (CET), chain A, ADP-ribosyltransferase, domains Ia, Ib, II and III
  DOMAIN (1)..(264): domain Ia, receptor binding domain
  DOMAIN (265)..(386): domain II, translocation domain
  DOMAIN (387)..(423): domain Ib
  DOMAIN (424)..(634): domain III, catalytic domain
VEDELNIFDECRSPCSLTPEPGKPIQSKLSIPSDVVLDEGVLYYSMTINDEQNDIKDEDKGESIITIGEFATVRATRHYVNQDAPFGVINLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPIDAIYNYITQQNCTLGDNWFGGSYETVAGTPKAITVKQGIEQKPVEQRIHFSKKNAMEALAAHRVCGVPLETLARSRKPRDLPDDLSCAYQAQNIVSLFVATRILFSHLDSVFTLNLDEQEPEVAERLSALRQINENNPGMVTQVLTVARQIYNDYVTHHPGLIPEQTSAGAQAADILSLFCPDADKPCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAEREARGVMLRVYIPRASLERFYRTNTPLENAERHITQVIGHSLPLRNEAFTGPESAGGEDETVIGWDMAIHAVAIPSTIPGNAYEELAIDEEAVAKEQSISAKPPYKEQKDELK

SEQ ID NO: 2 - protein, 364 aa, Artificial Sequence
  Description of Artificial Sequence:synthetic truncated cholera exotoxin (CET40), domains II, Ib and III, deleted domain Ia
ALAAHRVCGVPLETLARSRKPRDLPDDLSCAYQAQNIVSLFVATRILFSHLDSVFTLNLDEQEPEVAERLSALRQINENNPGMVTQVLTVARQIYNDYVTHHPGLIPEQTSAGAQAADILSLFCPDADKPCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAEREARGVMLRVYIPRASLERFYRTNTPLENAERHITQVIGHSLPLRNEAFTGPESAGGEDETVIGWDMAIHAVAIPSTIPGNAYEELAIDEEAVAKEQSISAKPPYKEQKDELK

SEQ ID NO: 3 - DNA, 1113 bp, Artificial Sequence
  Description of Artificial Sequence:synthetic nucleotide sequence encoding truncated cholera exotoxin (CET40)
  CDS (15)..(1109): CET40
ccggaggtcccgaggcacttgcagctcatcgtgtctgtggtgtgccattagaaaccttggcgcgcagtcggaagcctcgtgatttaccggatgatttatcatgtgcctatcaagcacagaatattgtgagtttatttgtcgcgacgcgtattttattctctcatctagatagcgtatttactctgaatcttgacgaacaagaaccagaggtggctgaacgtctaagtgctcttcgtcaaattaatgaaaataaccccggcatggttacacaggttttaaccgttgctcgccagatctataacgattatgtcactcaccatcccggattaattcctgagcaaaccagtgcgggtgcacaagctgccgatatcctctctttattttgcccagatgctgataagccttgtgtggcgtcaaacaacgatcaagctaatattaacattgagtctcgttctggtcgttcatatttgcctgaaaaccgtgcggtaatcacccctcaaggagtcacaaattggacttatcaggaactcgaagcaacacatcaagctctgactcgcgagggttatgtgttcgtgggttaccatggtacgaatcatgtcgctgcgcaaaccatagtgaatcgtattgcccctgttccgcgtggcaacaacactgaaaacgaggaaaagtggggcgggttatatgttgcaactcacgctgaagttgcccatggttatgctcgcatcaaagaagggacaggggagtatggacttccgacccgtgctgagcgtgaggctcgtggggtaatgctacgtgtgtatatccctcgtgcttcattggaacgtttttatcgcacgaatacacctttggaaaatgctgaaaggcatataacgcaagtgattggtcattctttgccattacgcaatgaagcatttactggtccagaaagtgcgggtggggaagacgaaactgtcattggctgggatatggcgattcatgcagttgcgattccttcgactattccggggaacgcttacgaagaattggcgattgatgaggaggctgttgctaaagagcaatcgattagcgcgaaaccaccttataaagagcaaaaagatgaactgaaataatgat

SEQ ID NO: 4 - protein, 4 aa, Artificial Sequence
  Description of Artificial Sequence:synthetic C-terminal sequence targeting toxin to endoplasmic reticulum, C-terminal residues of mutated Pseudomonas exotoxin A
KDEL

SEQ ID NO: 5 - protein, 5 aa, Artificial Sequence
  Description of Artificial Sequence:synthetic C-terminal residues of Pseudomonas exotoxin A, residues 609-613
REDLK

SEQ ID NO: 6 - protein, 4 aa, Artificial Sequence
  Description of Artificial Sequence:synthetic C-terminal residues of mutated Pseudomonas exotoxin A
REEL

SEQ ID NO: 7 - protein, 4 aa, Artificial Sequence
  Description of Artificial Sequence:synthetic C-terminal residues of mutated Pseudomonas exotoxin A
RDEL

SEQ ID NO: 8 - protein, 5 aa, Artificial Sequence
  Description of Artificial Sequence:synthetic carboxyl terminal residues of cholera exotoxin (CET)
KDELK

SEQ ID NO: 9 - protein, 4 aa, Artificial Sequence
  Description of Artificial Sequence:synthetic minimal cleavage site for furin, furin cleavage motif consensus sequence
  VARIANT (2)..(3): Xaa = any amino acid
RXXR

SEQ ID NO: 10 - protein, 4 aa, Artificial Sequence
  Description of Artificial Sequence:synthetic minimal cleavage site for furin, furin cleavage motif consensus sequence
  VARIANT (2): Xaa = any amino acid
  VARIANT (3): Xaa = Arg or Lys
RXXR

SEQ ID NO: 11 - protein, 4 aa, Artificial Sequence
  Description of Artificial Sequence:synthetic minimal cleavage site for furin, furin cleavage motif consensus sequence
  VARIANT (2): Xaa = any amino acid
RXRR

SEQ ID NO: 12 - protein, 4 aa, Artificial Sequence
  Description of Artificial Sequence:synthetic minimal cleavage site for furin, furin cleavage motif consensus sequence
  VARIANT (2): Xaa = any amino acid
RXKR

SEQ ID NO: 13 - protein, 363 aa, Artificial Sequence
  Description of Artificial Sequence:synthetic Pseudomonas exotoxin A (PE) 40 kD fragment, PE40, domains II, Ib and III
PEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 14 - protein, 371 aa, Artificial Sequence
  Description of Artificial Sequence:synthetic Vibrio cholerae 40 kD fragment of cholix toxin (CholExo), domains II, Ib and III
SKKNAMEALAAHRVCGVPLETLARSRKPRDLPDDLSCAYQAQNIVSLFVATRILFSHLDSVFTLNLDEQEPEVAERLSALRQINENNPGMVTQVLTVARQIYNDYVTHHPGLIPEQTSAGAQAADILSLFCPDADKPCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAEREARGVMLRVYIPRASLERFYRTNTPLENAERHITQVIGHSLPLRNEAFTGPESAGGEDETVIGWDMAIHAVAIPSTIPGNAYEELAIDEEAVAKEQSISAKPPYKEQKDELK

SEQ ID NO: 15 - DNA, 5173 bp, Artificial Sequence
  Description of Artificial Sequence:synthetic plasmid HB21-PE38 encoding chimeric anti-transferrin receptor single chain Fv antibody HB21 fragment fused to 38 kD form of Pseudomonas exotoxin A (PE38)
  CDS (79)..(1863): HB21-PE38
  CDS complement (3071..3532): CAT antibiotic resistance gene product
taatacgactcactatagggagaccacaacggtttccctctagaaataattttgtttaactttaagaaggagatatatatggaggtgcagctggtggagtctggggctgagcttgtgaggccaggggccttagtcaagttgtcctgcaaagcttctggcttcaacattaaagactcctatatgcactgggtgaatcagaggcctgaacagggcctggagtggattggatggattgatcctgagactggtaatactatatatgacccgaagttccagggcaaggccagtataactgcagactcatcctccaacacagcctacctgcagctcaccagcctgacatctgaggacactgccgtctattactgtgctagaggtagtatctactggtacttcgatgtctggggcgcagggaccacggtcaccgtctcctcaggcggaggcggatccggtggtggcggctctggaggtggcggcagcaatattgtaatgacccagtctccatcctccctggctatgtcagtaggacagaaggtcactatgagctgcaagtccagtcagagccttttaaatagtagcaatcaaaagaactctttggcctggtaccagcagaaaccaggacagtctcctaaacttctgctatactttgcatccactaggggatctggggtccctgatcgcttcataggcagtggatctgggacagatttcactcttaccatcagcagtgtgcaggctgaagacctggcagattacttctgtcagcaacattatagcactcctctcacgttcggtgctgggaccaagctggagataaaagctttcggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaactgccgcgaccggccggctcccttcgcaggagccggccttctcggggcctggccatacatcaggttttcctgatgccagcccaatcgaatatgaattcggctgctaacaaagcccgaaaggaagctgagttggctgctgccaccgctgagcaataactagcataaccccttggggcctctaaacgggtcttgaggggttttttgctgaaaggaggaactatatccggatcgagatcaattctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttggggatcctctagagttgcatgcctgcaggtccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaaccggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcatcagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccactttagcttccttagctcctgaaaatctcggtaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgactctagaggatccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgaccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 16 - protein, 594 aa, Artificial Sequence
  Description of Artificial Sequence:synthetic HB21-PE38 chimeric anti-transferrin receptor single chain Fv antibody HB21 fragment fused to 38 kD form of Pseudomonas exotoxin A (PE38)
MEVQLVESGAELVRPGALVKLSCKASGFNIKDSYMHWVNQRPEQGLEWIGWIDPETGNTIYDPKFQGKASITADSSSNTAYLQLTSLTSEDTAVYYCARGSIYWYFDVWGAGTTVTVSSGGGGSGGGGSGGGGSNIVMTQSPSSLAMSVGQKVTMSCKSSQSLLNSSNQKNSLAWYQQKPGQSPKLLLYFASTRGSGVPDRFIGSGSGTDFTLTISSVQAEDLADYFCQQHYSTPLTFGAGTKLEIKAFGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 17 - protein, 153 aa, Artificial Sequence
  Description of Artificial Sequence:synthetic CAT antibiotic resistance gene product
MNAHPEFRMAMKDGELMIWDSVHPCYTVFHEQTETFSSLWSEYHDDFRQFLHIYSQDVACYGENLAYFPKGFIENMFFVSANPWVSFTGFDLNVANMDNFFAPVFTMGKYYTQGDKVLMPLAIQVHHAVCDGFHVGRMLNELQQYCDEWQGGA

SEQ ID NO: 18 - DNA, 5156 bp, Artificial Sequence
  Description of Artificial Sequence:synthetic plasmid HB21-CET40 (HB21scFV-CET40) encoding chimeric anti- transferrin receptor single chain Fv antibody HB21 fragment fused to 40 kD truncated form of Vibrio cholerae cholera exotoxin (CET40)
  CDS (79)..(1932): HB21-CET40
  CDS complement (3052..3513): CAT antibiotic resistance gene product
taatacgactcactatagggagaccacaacggtttccctctagaaataattttgtttaactttaagaaggagatatatatggaggtgcagctggtggagtctggggctgagcttgtgaggccaggggccttagtcaagttgtcctgcaaagcttctggcttcaacattaaagactcctatatgcactgggtgaatcagaggcctgaacagggcctggagtggattggatggattgatcctgagactggtaatactatatatgacccgaagttccagggcaaggccagtataactgcagactcatcctccaacacagcctacctgcagctcaccagcctgacatctgaggacactgccgtctattactgtgctagaggtagtatctactggtacttcgatgtctggggcgcagggaccacggtcaccgtctcctcaggcggaggcggatccggtggtggcggctctggaggtggcggcagcaatattgtaatgacccagtctccatcctccctggctatgtcagtaggacagaaggtcactatgagctgcaagtccagtcagagccttttaaatagtagcaatcaaaagaactctttggcctggtaccagcagaaaccaggacagtctcctaaacttctgctatactttgcatccactaggggatctggggtccctgatcgcttcataggcagtggatctgggacagatttcactcttaccatcagcagtgtgcaggctgaagacctggcagattacttctgtcagcaacattatagcactcctctcacgttcggtgctgggaccaagctggagataaaagcttccggaggtcccgaggcacttgcagctcatcgtgtctgtggtgtgccattagaaaccttggcgcgcagtcggaagcctcgtgatttaccggatgatttatcatgtgcctatcaagcacagaatattgtgagtttatttgtcgcgacgcgtattttattctctcatctagatagcgtatttactctgaatcttgacgaacaagaaccagaggtggctgaacgtctaagtgctcttcgtcaaattaatgaaaataaccccggcatggttacacaggttttaaccgttgctcgccagatctataacgattatgtcactcaccatcccggattaattcctgagcaaaccagtgcgggtgcacaagctgccgatatcctctctttattttgcccagatgctgataagccttgtgtggcgtcaaacaacgatcaagctaatattaacattgagtctcgttctggtcgttcatatttgcctgaaaaccgtgcggtaatcacccctcaaggagtcacaaattggacttatcaggaactcgaagcaacacatcaagctctgactcgcgagggttatgtgttcgtgggttaccatggtacgaatcatgtcgctgcgcaaaccatagtgaatcgtattgcccctgttccgcgtggcaacaacactgaaaacgaggaaaagtggggcgggttatatgttgcaactcacgctgaagttgcccatggttatgctcgcatcaaagaagggacaggggagtatggacttccgacccgtgctgagcgtgaggctcgtggggtaatgctacgtgtgtatatccctcgtgcttcattggaacgtttttatcgcacgaatacacctttggaaaatgctgaaaggcatataacgcaagtgattggtcattctttgccattacgcaatgaagcatttactggtccagaaagtgcgggtggggaagacgaaactgtcattggctgggatatggcgattcatgcagttgcgattccttcgactattccggggaacgcttacgaagaattggcgattgatgaggaggctgttgctaaagagcaatcgattagcgcgaaaccaccttataaagagcaaaaagatgaactgaaataatgatgaattcggctgctaacaaagcccgaaaggaagctgagttggctgctgccaccgctgagcaataactagcataaccccttggggcctctaaacgggtcttgaggggttttttgctgaaaggaggaactatatccggatcgagatcaattctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttggggatcctctagagttgcatgcctgcaggtccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaaccggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcatcagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccactttagcttccttagctcctgaaaatctcggtaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgactctagaggatccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgaccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaatta

SEQ ID NO: 19 - protein, 617 aa, Artificial Sequence
  Description of Artificial Sequence:synthetic HB21-CET40 (HB21scFV-CET40) chimeric anti-transferrin receptor single chain Fv antibody HB21 fragment fused to 40 kD truncated form of Vibrio cholerae cholera exotoxin (CET40)
MEVQLVESGAELVRPGALVKLSCKASGFNIKDSYMHWVNQRPEQGLEWIGWIDPETGNTIYDPKFQGKASITADSSSNTAYLQLTSLTSEDTAVYYCARGSIYWYFDVWGAGTTVTVSSGGGGSGGGGSGGGGSNIVMTQSPSSLAMSVGQKVTMSCKSSQSLLNSSNQKNSLAWYQQKPGQSPKLLLYFASTRGSGVPDRFIGSGSGTDFTLTISSVQAEDLADYFCQQHYSTPLTFGAGTKLEIKASGGPEALAAHRVCGVPLETLARSRKPRDLPDDLSCAYQAQNIVSLFVATRILFSHLDSVFTLNLDEQEPEVAERLSALRQINENNPGMVTQVLTVARQIYNDYVTHHPGLIPEQTSAGAQAADILSLFCPDADKPCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAEREARGVMLRVYIPRASLERFYRTNTPLENAERHITQVIGHSLPLRNEAFTGPESAGGEDETVIGWDMAIHAVAIPSTIPGNAYEELAIDEEAVAKEQSISAKPPYKEQKDELK

SEQ ID NO: 20 - protein, 8 aa, Artificial Sequence
  Description of Artificial Sequence:synthetic 8 residue leader sequence, N-terminal 6 histidine tag flanked by methionine residues
MHHHHHHM

SEQ ID NO: 21 - DNA, 1854 bp, Artificial Sequence
  Description of Artificial Sequence:synthetic plasmid HB21-CET40 (HB21scFV-CET40) coding region for chimeric anti-transferrin receptor single chain Fv antibody HB21 fragment fused to 40 kD truncated form of Vibrio cholerae cholera exotoxin (CET40)
  CDS (1)..(1854): HB21-CET40
atggaggtgcagctggtggagtctggggctgagcttgtgaggccaggggccttagtcaagttgtcctgcaaagcttctggcttcaacattaaagactcctatatgcactgggtgaatcagaggcctgaacagggcctggagtggattggatggattgatcctgagactggtaatactatatatgacccgaagttccagggcaaggccagtataactgcagactcatcctccaacacagcctacctgcagctcaccagcctgacatctgaggacactgccgtctattactgtgctagaggtagtatctactggtacttcgatgtctggggcgcagggaccacggtcaccgtctcctcaggcggaggcggatccggtggtggcggctctggaggtggcggcagcaatattgtaatgacccagtctccatcctccctggctatgtcagtaggacagaaggtcactatgagctgcaagtccagtcagagccttttaaatagtagcaatcaaaagaactctttggcctggtaccagcagaaaccaggacagtctcctaaacttctgctatactttgcatccactaggggatctggggtccctgatcgcttcataggcagtggatctgggacagatttcactcttaccatcagcagtgtgcaggctgaagacctggcagattacttctgtcagcaacattatagcactcctctcacgttcggtgctgggaccaagctggagataaaagcttccggaggtcccgaggcacttgcagctcatcgtgtctgtggtgtgccattagaaaccttggcgcgcagtcggaagcctcgtgatttaccggatgatttatcatgtgcctatcaagcacagaatattgtgagtttatttgtcgcgacgcgtattttattctctcatctagatagcgtatttactctgaatcttgacgaacaagaaccagaggtggctgaacgtctaagtgctcttcgtcaaattaatgaaaataaccccggcatggttacacaggttttaaccgttgctcgccagatctataacgattatgtcactcaccatcccggattaattcctgagcaaaccagtgcgggtgcacaagctgccgatatcctctctttattttgcccagatgctgataagccttgtgtggcgtcaaacaacgatcaagctaatattaacattgagtctcgttctggtcgttcatatttgcctgaaaaccgtgcggtaatcacccctcaaggagtcacaaattggacttatcaggaactcgaagcaacacatcaagctctgactcgcgagggttatgtgttcgtgggttaccatggtacgaatcatgtcgctgcgcaaaccatagtgaatcgtattgcccctgttccgcgtggcaacaacactgaaaacgaggaaaagtggggcgggttatatgttgcaactcacgctgaagttgcccatggttatgctcgcatcaaagaagggacaggggagtatggacttccgacccgtgctgagcgtgaggctcgtggggtaatgctacgtgtgtatatccctcgtgcttcattggaacgtttttatcgcacgaatacacctttggaaaatgctgaaaggcatataacgcaagtgattggtcattctttgccattacgcaatgaagcatttactggtccagaaagtgcgggtggggaagacgaaactgtcattggctgggatatggcgattcatgcagttgcgattccttcgactattccggggaacgcttacgaagaattggcgattgatgaggaggctgttgctaaagagcaatcgattagcgcgaaaccaccttataaagagcaaaaagatgaactgaaataa

SEQ ID NO: 22 - skipped

SEQ ID NO: 23 - protein, 365 aa, Artificial Sequence
  Description of Artificial Sequence:synthetic Vibrio cholerae cholix toxin (CT) amino acids 270-634
SALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 24 - protein, 365 aa, Artificial Sequence
  Description of Artificial Sequence:synthetic Vibrio cholerae strain 1587 cholera exotoxin (CET) amino acids 270-634, domains II and II
EALAAHRVCGVPLETLARSRKPRDLPDDLSCAYQAQNIVSLFVATRILFSHLDSVFTLNLDEQEPEVAERLSALRQINENNPGMVTQVLTVARQIYNDYVTHHPGLIPEQTSAGAQAADILSLFCPDADKPCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAEREARGVMLRVYIPRASLERFYRTNTPLENAERHITQVIGHSLPLRNEAFTGPESAGGEDETVIGWDMAIHAVAIPSTIPGNAYEELAIDEEAVAKEQSISAKPPYKEQKDELK

SEQ ID NO: 25 - protein, 357 aa, Artificial Sequence
  Description of Artificial Sequence:synthetic Pseudomonas exotoxin A domains II and III (PE40)
AALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 26 - protein, 15 aa, Artificial Sequence
  Description of Artificial Sequence:synthetic glycine-serine linker
GGGGSGGGGSGGGGS

SEQ ID NO: 27 - protein, 6 aa, Artificial Sequence
  Description of Artificial Sequence:synthetic short connector sequence
ASGGPE

SEQ ID NO: 28 - protein, 9 aa, Artificial Sequence
  Description of Artificial Sequence:synthetic cholera toxin NAD domain
GGEDETVIG

SEQ ID NO: 29 - protein, 9 aa, Artificial Sequence
  Description of Artificial Sequence:synthetic terminal sequence of Pseudomonas exotoxin A (PE)
GGRLETILG

SEQ ID NO: 30 - protein, 345 aa, Artificial Sequence
  Description of Artificial Sequence:synthetic Pseudomonas exotoxin A fragment PE38
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 31 - protein, 634 aa, Vibrio cholerae
  mature cholix toxin
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 32 - protein, 318 aa, Artificial Sequence
  Description of Artificial Sequence:synthetic Pseudomonas exotoxin A fragment PE35
MWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 33 - DNA, 1095 bp, Artificial Sequence
  Description of Artificial Sequence:synthetic sequence encoding cholera exotoxin fragment CET40
gaggcacttgcagctcatcgtgtctgtggtgtgccattagaaaccttggcgcgcagtcggaagcctcgtgatttaccggatgatttatcatgtgcctatcaagcacagaatattgtgagtttatttgtcgcgacgcgtattttattctctcatctagatagcgtatttactctgaatcttgacgaacaagaaccagaggtggctgaacgtctaagtgctcttcgtcaaattaatgaaaataaccccggcatggttacacaggttttaaccgttgctcgccagatctataacgattatgtcactcaccatcccggattaattcctgagcaaaccagtgcgggtgcacaagctgccgatatcctctctttattttgcccagatgctgataagccttgtgtggcgtcaaacaacgatcaagctaatattaacattgagtctcgttctggtcgttcatatttgcctgaaaaccgtgcggtaatcacccctcaaggagtcacaaattggacttatcaggaactcgaagcaacacatcaagctctgactcgcgagggttatgtgttcgtgggttaccatggtacgaatcatgtcgctgcgcaaaccatagtgaatcgtattgcccctgttccgcgtggcaacaacactgaaaacgaggaaaagtggggcgggttatatgttgcaactcacgctgaagttgcccatggttatgctcgcatcaaagaagggacaggggagtatggacttccgacccgtgctgagcgtgaggctcgtggggtaatgctacgtgtgtatatccctcgtgcttcattggaacgtttttatcgcacgaatacacctttggaaaatgctgaaaggcatataacgcaagtgattggtcattctttgccattacgcaatgaagcatttactggtccagaaagtgcgggtggggaagacgaaactgtcattggctgggatatggcgattcatgcagttgcgattccttcgactattccggggaacgcttacgaagaattggcgattgatgaggaggctgttgctaaagagcaatcgattagcgcgaaaccaccttataaagagcaaaaagatgaactgaaa

SEQ ID NO: 34 - DNA, 1845 bp, Artificial Sequence
  Description of Artificial Sequence:synthetic plasmid HB21-PE40 coding region for chimeric anti-transferrin receptor single chain Fv antibody HB21 fragment fused to 40 kD form of Pseudomonas exotoxin A (PE40)
  CDS (1)..(1845): HB21scFv-PE40
atggaggtgcagctggtggagtctggggctgagcttgtgaggccaggggccttagtcaagttgtcctgcaaagcttctggcttcaacattaaagactcctatatgcactgggtgaatcagaggcctgaacagggcctggagtggattggatggattgatcctgagactggtaatactatatatgacccgaagttccagggcaaggccagtataactgcagactcatcctccaacacagcctacctgcagctcaccagcctgacatctgaggacactgccgtctattactgtgctagaggtagtatctactggtacttcgatgtctggggcgcagggaccacggtcaccgtctcctcaggcggaggcggatccggtggtggcggctctggaggtggcggcagcaatattgtaatgacccagtctccatcctccctggctatgtcagtaggacagaaggtcactatgagctgcaagtccagtcagagccttttaaatagtagcaatcaaaagaactctttggcctggtaccagcagaaaccaggacagtctcctatacttctgctatacttcgcatccactaggggatctggggtccctgatcgcttcataggcagtggatctgggacagatttcactcttaccatcagcagtgtgcaggctgaagacctggcagattacttctgtcagcaacattatagcactcctctcacgttcggtgctgggaccaagctggagataaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 35 - protein, 614 aa, Artificial Sequence
  Description of Artificial Sequence:synthetic HB21-PE40 chimeric anti-transferrin receptor single chain Fv antibody HB21 fragment fused to 40 kD form of Pseudomonas exotoxin A (PE40)
MEVQLVESGAELVRPGALVKLSCKASGFNIKDSYMHWVNQRPEQGLEWIGWIDPETGNTIYDPKFQGKASITADSSSNTAYLQLTSLTSEDTAVYYCARGSIYWYFDVWGAGTTVTVSSGGGGSGGGGSGGGGSNIVMTQSPSSLAMSVGQKVTMSCKSSQSLLNSSNQKNSLAWYQQKPGQSPILLLYFASTRGSGVPDRFIGSGSGTDFTLTISSVQAEDLADYFCQQHYSTPLTFGAGTKLEIKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 36 - protein, 218 aa, Artificial Sequence
  Description of Artificial Sequence:synthetic truncated cholera exotoxin (CET) domain III
SGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAEREARGVMLRVYIPRASLERFYRTNTPLENAERHITQVIGHSLPLRNEAFTGPESAGGEDETVIGWDMAIHAVAIPSTIPGNAYEELAIDEEAVAKEQSISAKPPYKEQKDELK